SEQUENCE LISTING (49 sequences)

SEQ ID NO: 1 - DNA, 9045 bp, Homo sapiens
  CDS (1)..(9042)
atggcgccgccgccgccgcccgtgctgcccgtgctgctgctcctggccgccgccgccgccctgccggcgatggggctgcgagcggccgcctgggagccgcgcgtacccggcgggacccgcgccttcgccctccggcccggctgtacctacgcggtgggcgccgcttgcacgccccgggcgccgcgggagctgctggacgtgggccgcgatgggcggctggcaggacgtcggcgcgtctcgggcgcggggcgcccgctgccgctgcaagtccgcttggtggcccgcagtgccccgacggcgctgagccgccgcctgcgggcgcgcacgcaccttcccggctgcggagcccgtgcccggctctgcggaaccggtgcccggctctgcggggcgctctgcttccccgtccccggcggctgcgcggccgcgcagcattcggcgctcgcagctccgaccaccttacccgcctgccgctgcccgccgcgccccaggccccgctgtcccggccgtcccatctgcctgccgccgggcggctcggtccgcctgcgtctgctgtgcgccctgcggcgcgcggctggcgccgtccgggtgggactggcgctggaggccgccaccgcggggacgccctccgcgtcgccatccccatcgccgcccctgccgccgaacttgcccgaagcccgggcggggccggcgcgacgggcccggcggggcacgagcggcagagggagcctgaagtttccgatgcccaactaccaggtggcgttgtttgagaacgaaccggcgggcaccctcatcctccagctgcacgcgcactacaccatcgagggcgaggaggagcgcgtgagctattacatggaggggctgttcgacgagcgctcccggggctacttccgaatcgactctgccacgggcgccgtgagcacggacagcgtactggaccgcgagaccaaggagacgcacgtcctcagggtgaaagccgtggactacagtacgccgccgcgctcggccaccacctacatcactgtcttggtcaaagacaccaacgaccacagcccggtcttcgagcagtcggagtaccgcgagcgcgtgcgggagaacctggaggtgggctacgaggtgctgaccatccgcgccagcgaccgcgactcgcccatcaacgccaacttgcgttaccgcgtgttggggggcgcgtgggacgtcttccagctcaacgagagctctggcgtggtgagcacacgggcggtgctggaccgggaggaggcggccgagtaccagctcctggtggaggccaacgaccaggggcgcaatccgggcccgctcagtgccacggccaccgtgtacatcgaggtggaggacgagaacgacaactacccccagttcagcgagcagaactacgtggtccaggtgcccgaggacgtggggctcaacacggctgtgctgcgagtgcaggccacggaccgggaccagggccagaacgcggccattcactacagcatcctcagcgggaacgtggccggccagttctacctgcactcgctgagcgggatcctggatgtgatcaaccccttggatttcgaggatgtccagaaatactcgctgagcattaaggcccaggatgggggccggcccccgctcatcaattcttcaggggtggtgtctgtgcaggtgctggatgtcaacgacaacgagcctatctttgtgagcagccccttccaggccacggtgctggagaatgtgcccctgggctaccccgtggtgcacattcaggcggtggacgcggactctggagagaacgcccggctgcactatcgcctggtggacacggcctccacctttctggggggcggcagcgctgggcctaagaatcctgcccccacccctgacttccccttccagatccacaacagctccggttggatcacagtgtgtgccgagctggaccgcgaggaggtggagcactacagcttcggggtggaggcggtggaccacggctcgccccccatgagctcctccaccagcgtgtccatcacggtgctggacgtgaatgacaacgacccggtgttcacgcagcccacctacgagcttcgtctgaatgaggatgcggccgtggggagcagcgtgctgaccctgcaggcccgcgaccgtgacgccaacagtgtgattacctaccagctcacaggcggcaacacccggaaccgctttgcactcagcagccagagagggggcggcctcatcaccctggcgctacctctggactacaagcaggagcagcagtacgtgctggcggtgacagcatccgacggcacacggtcgcacactgcgcatgtcctaatcaacgtcactgatgccaacacccacaggcctgtctttcagagctcccattacacagtgagtgtcagtgaggacaggcctgtgggcacctccattgctaccctcagtgccaacgatgaggacacaggagagaatgcccgcatcacctacgtgattcaggaccccgtgccgcagttccgcattgaccccgacagtggcaccatgtacaccatgatggagctggactatgagaaccaggtcgcctacacgctgaccatcatggcccaggacaacggcatcccgcagaaatcagacaccaccaccctagagatcctcatcctcgatgccaatgacaatgcaccccagttcctgtgggatttctaccagggttccatctttgaggatgctccaccctcgaccagcatcctccaggtctctgccacggaccgggactcaggtcccaatgggcgtctgctgtacaccttccagggtggggacgacggcgatggggacttctacatcgagcccacgtccggtgtgattcgcacccagcgccggctggaccgggagaatgtggccgtgtacaacctttgggctctggctgtggatcggggcagtcccactccccttagcgcctcggtagaaatccaggtgaccatcttggacattaatgacaatgcccccatgtttgagaaggacgaactggagctgtttgttgaggagaacaacccagtggggtcggtggtggcaaagattcgtgctaacgaccctgatgaaggccctaatgcccagatcatgtatcagattgtggaaggggacatgcggcatttcttccagctggacctgctcaacggggacctgcgtgccatggtggagctggactttgaggtccggcgggagtatgtgctggtggtgcaggccacgtcggctccgctggtgagccgagccacggtgcacatccttctcgtggaccagaatgacaacccgcctgtgctgcccgacttccagatcctcttcaacaactatgtcaccaacaagtccaacagtttccccaccggcgtgatcggctgcatcccggcccatgaccccgacgtgtcagacagcctcaactacaccttcgtgcagggcaacgagctgcgcctgttgctgctggaccccgccacgggcgaactgcagctcagccgcgacctggacaacaaccggccgctggaggcgctcatggaggtgtctgtgtctgatggcatccacagcgtcacggccttctgcaccctgcgtgtcaccatcatcacggacgacatgctgaccaacagcatcactgtccgcctggagaacatgtcccaggagaagttcctgtccccgctgctggccctcttcgtggagggggtggccgccgtgctgtccaccaccaaggacgacgtcttcgtcttcaacgtccagaacgacaccgacgtcagctccaacatcctgaacgtgaccttctcggcgctgctgcctggcggcgtccgcggccagttcttcccgtcggaggacctgcaggagcagatctacctgaatcggacgctgctgaccaccatctccacgcagcgcgtgctgcccttcgacgacaacatctgcctgcgcgagccctgcgagaactacatgaagtgcgtgtccgttctgcgattcgacagctccgcgcccttcctcagctccaccaccgtgctcttccggcccatccaccccatcaacggcctgcgctgccgctgcccgcccggcttcaccggcgactactgcgagacggagatcgacctctgctactccgacccgtgcggcgccaacggccgctgccgcagccgcgagggcggctacacctgcgagtgcttcgaggacttcactggagagcactgtgaggtggatgcccgctcaggccgctgtgccaacggggtgtgcaagaacgggggcacctgcgtgaacctgctcatcggcggcttccactgcgtgtgtcctcctggcgagtatgagaggccctactgtgaggtgaccaccaggagcttcccgccccagtccttcgtcaccttccggggcctgagacagcgcttccacttcaccatctccctcacgtttgccactcaggaaaggaacggcttgcttctctacaacggccgcttcaatgagaagcacgacttcatcgccctggagatcgtggacgagcaggtgcagctcaccttctctgcaggcgagacaacaacgaccgtggcaccgaaggttcccagtggtgtgagtgacgggcggtggcactctgtgcaggtgcagtactacaacaagcccaatattggccacctgggcctgccccatgggccgtccggggaaaagatggccgtggtgacagtggatgattgtgacacaaccatggctgtgcgctttggaaaggacatcgggaactacagctgcgctgcccagggcactcagaccggctccaagaagtccctggatctgaccggccctctactcctggggggtgtccccaacctgccagaagacttcccagtgcacaaccggcagttcgtgggctgcatgcggaacctgtcagtcgacggcaaaaatgtggacatggccggattcatcgccaacaatggcacccgggaaggctgcgctgctcggaggaacttctgcgatgggaggcggtgtcagaatggaggcacctgtgtcaacaggtggaatatgtatctgtgtgagtgtccactccgattcggcgggaagaactgtgagcaagccatgcctcacccccagctcttcagcggtgagagcgtcgtgtcctggagtgacctgaacatcatcatctctgtgccctggtacctggggctcatgttccggacccggaaggaggacagcgttctgatggaggccaccagtggtgggcccaccagctttcgcctccagatcctgaacaactacctccagtttgaggtgtcccacggcccctccgatgtggagtccgtgatgctgtccgggttgcgggtgaccgacggggagtggcaccacctgctgatcgagctgaagaatgttaaggaggacagtgagatgaagcacctggtcaccatgaccttggactatgggatggaccagaacaaggcagatatcgggggcatgcttcccgggctgacggtaaggagcgtggtggtcggaggcgcctctgaagacaaggtctccgtgcgccgtggattccgaggctgcatgcagggagtgaggatgggggggacgcccaccaacgtcgccaccctgaacatgaacaacgcactcaaggtcagggtgaaggacggctgtgatgtggacgacccctgtacctcgagcccctgtccccccaatagccgctgccacgacgcctgggaggactacagctgcgtctgtgacaaagggtaccttggaataaactgtgtggatgcctgtcacctgaacccctgcgagaacatgggggcctgcgtgcgctcccccggctccccgcagggctacgtgtgcgagtgtgggcccagtcactacgggccgtactgtgagaacaaactcgaccttccgtgccccagaggctggtgggggaaccccgtctgtggaccctgccactgtgccgtcagcaaaggctttgatcccgactgtaataagaccaacggccagtgccaatgcaaggagaattactacaagctcctagcccaggacacctgtctgccctgcgactgcttcccccatggctcccacagccgcacttgcgacatggccaccgggcagtgtgcctgcaagcccggcgtcatcggccgccagtgcaaccgctgcgacaacccgtttgccgaggtcaccacgctcggctgtgaagtgatctacaatggctgtcccaaagcatttgaggccggcatctggtggccacagaccaagttcgggcagccggctgcggtgccatgccctaagggatccgttggaaatgcggtccgacactgcagcggggagaagggctggctgcccccagagctctttaactgtaccaccatctccttcgtggacctcagggccatgaatgagaagctgagccgcaatgagacgcaggtggacggcgccagggccctgcagctggtgagggcgctgcgcagtgctacacagcacacgggcacgctctttggcaatgacgtgcgcacggcctaccagctgctgggccacgtccttcagcacgagagctggcagcagggcttcgacctggcagccacgcaggacgccgactttcacgaggacgtcatccactcgggcagcgccctcctggccccagccaccagggcggcgtgggagcagatccagcggagcgagggcggcacggcacagctgctccggcgcctcgagggctacttcagcaacgtggcacgcaacgtgcggcggacgtacctgcggcccttcgtcatcgtcaccgccaacatgattcttgctgtcgacatctttgacaagttcaactttacgggagccagggtcccgcgattcgacaccatccatgaagagttccccagggagctggagtcctccgtctccttcccagccgacttcttcagaccacctgaagaaaaagaaggccccctgctgaggccggctggccggaggaccaccccgcagaccacgcgcccggggcctggcaccgagagggaggccccgatcagcaggcggaggcgacaccctgatgacgctggccagttcgccgtcgctctggtcatcatttaccgcaccctggggcagctcctgcccgagcgctacgaccccgaccgtcgcagcctccggttgcctcaccggcccatcattaataccccgatggtgagcacgctggtgtacagcgagggggctccgctcccgagacccctggagaggcccgtcctggtggagttcgccctgctggaggtggaggagcgaaccaagcctgtctgcgtgttctggaaccactccctggccgttggtgggacgggagggtggtctgcccggggctgcgagctcctgtccaggaaccggacacatgtcgcctgccagtgcagccacacagccagctttgcggtgctcatggatatctccaggcgtgagaacggggaggtcctgcctctgaagattgtcacctatgccgctgtgtccttgtcactggcagccctgctggtggccttcgtcctcctgagcctggtccgcatgctgcgctccaacctgcacagcattcacaagcacctcgccgtggcgctcttcctctctcagctggtgttcgtgattgggatcaaccagacggaaaacccgtttctgtgcacagtggttgccatcctcctccactacatctacatgagcacctttgcctggaccctcgtggagagcctgcatgtctaccgcatgctgaccgaggtgcgcaacatcgacacggggcccatgcggttctactacgtcgtgggctggggcatcccggccattgtcacaggactggcggtcggcctggacccccagggctacgggaaccccgacttctgctggctgtcgcttcaagacaccctgatttggagctttgcggggcccatcggagctgttataatcatcaacacagtcacttctgtcctatctgcaaaggtttcctgccaaagaaagcaccattattatgggaaaaaagggatcgtctccctgctgaggaccgcattcctcctgctgctgctcatcagcgccacctggctgctggggctgctggctgtgaaccgcgatgcactgagctttcactacctcttcgccatcttcagcggcttacagggccccttcgtcctccttttccactgcgtgctcaaccaggaggtccggaagcacctgaagggcgtgctcggcgggaggaagctgcacctggaggactccgccaccaccagggccaccctgctgacgcgctccctcaactgcaacaccaccttcggtgacgggcctgacatgctgcgcacagacttgggcgagtccaccgcctcgctggacagcatcgtcagggatgaagggatccagaagctcggcgtgtcctctgggctggtgaggggcagccacggagagccagacgcgtccctcatgcccaggagctgcaaggatccccctggccacgattccgactcagatagcgagctgtccctggatgagcagagcagctcttacgcctcctcacactcgtcagacagcgaggacgatggggtgggagctgaggaaaaatgggacccggccaggggcgccgtccacagcacccccaaaggggacgctgtggccaaccacgttccggccggctggcccgaccagagcctggctgagagtgacagtgaggaccccagcggcaagccccgcctgaaggtggagaccaaggtcagcgtggagctgcaccgcgaggagcagggcagtcaccgtggagagtaccccccggaccaggagagcgggggcgcagccaggcttgctagcagccagcccccagagcagaggaaaggcatcttgaaaaataaagtcacctacccgccgccgctgacgctgacggagcagacgctgaagggccggctccgggagaagctggccgactgtgagcagagccccacatcctcgcgcacgtcttccctgggctctggcggccccgactgcgccatcacagtcaagagccctgggagggagccggggcgtgaccacctcaacggggtggccatgaatgtgcgcactgggagcgcccaggccgatggctccgactctgagaaaccgtga

SEQ ID NO: 2 - protein, 3014 aa, Homo sapiens
MAPPPPPVLPVLLLLAAAAALPAMGLRAAAWEPRVPGGTRAFALRPGCTYAVGAACTPRAPRELLDVGRDGRLAGRRRVSGAGRPLPLQVRLVARSAPTALSRRLRARTHLPGCGARARLCGTGARLCGALCFPVPGGCAAAQHSALAAPTTLPACRCPPRPRPRCPGRPICLPPGGSVRLRLLCALRRAAGAVRVGLALEAATAGTPSASPSPSPPLPPNLPEARAGPARRARRGTSGRGSLKFPMPNYQVALFENEPAGTLILQLHAHYTIEGEEERVSYYMEGLFDERSRGYFRIDSATGAVSTDSVLDRETKETHVLRVKAVDYSTPPRSATTYITVLVKDTNDHSPVFEQSEYRERVRENLEVGYEVLTIRASDRDSPINANLRYRVLGGAWDVFQLNESSGVVSTRAVLDREEAAEYQLLVEANDQGRNPGPLSATATVYIEVEDENDNYPQFSEQNYVVQVPEDVGLNTAVLRVQATDRDQGQNAAIHYSILSGNVAGQFYLHSLSGILDVINPLDFEDVQKYSLSIKAQDGGRPPLINSSGVVSVQVLDVNDNEPIFVSSPFQATVLENVPLGYPVVHIQAVDADSGENARLHYRLVDTASTFLGGGSAGPKNPAPTPDFPFQIHNSSGWITVCAELDREEVEHYSFGVEAVDHGSPPMSSSTSVSITVLDVNDNDPVFTQPTYELRLNEDAAVGSSVLTLQARDRDANSVITYQLTGGNTRNRFALSSQRGGGLITLALPLDYKQEQQYVLAVTASDGTRSHTAHVLINVTDANTHRPVFQSSHYTVSVSEDRPVGTSIATLSANDEDTGENARITYVIQDPVPQFRIDPDSGTMYTMMELDYENQVAYTLTIMAQDNGIPQKSDTTTLEILILDANDNAPQFLWDFYQGSIFEDAPPSTSILQVSATDRDSGPNGRLLYTFQGGDDGDGDFYIEPTSGVIRTQRRLDRENVAVYNLWALAVDRGSPTPLSASVEIQVTILDINDNAPMFEKDELELFVEENNPVGSVVAKIRANDPDEGPNAQIMYQIVEGDMRHFFQLDLLNGDLRAMVELDFEVRREYVLVVQATSAPLVSRATVHILLVDQNDNPPVLPDFQILFNNYVTNKSNSFPTGVIGCIPAHDPDVSDSLNYTFVQGNELRLLLLDPATGELQLSRDLDNNRPLEALMEVSVSDGIHSVTAFCTLRVTIITDDMLTNSITVRLENMSQEKFLSPLLALFVEGVAAVLSTTKDDVFVFNVQNDTDVSSNILNVTFSALLPGGVRGQFFPSEDLQEQIYLNRTLLTTISTQRVLPFDDNICLREPCENYMKCVSVLRFDSSAPFLSSTTVLFRPIHPINGLRCRCPPGFTGDYCETEIDLCYSDPCGANGRCRSREGGYTCECFEDFTGEHCEVDARSGRCANGVCKNGGTCVNLLIGGFHCVCPPGEYERPYCEVTTRSFPPQSFVTFRGLRQRFHFTISLTFATQERNGLLLYNGRFNEKHDFIALEIVDEQVQLTFSAGETTTTVAPKVPSGVSDGRWHSVQVQYYNKPNIGHLGLPHGPSGEKMAVVTVDDCDTTMAVRFGKDIGNYSCAAQGTQTGSKKSLDLTGPLLLGGVPNLPEDFPVHNRQFVGCMRNLSVDGKNVDMAGFIANNGTREGCAARRNFCDGRRCQNGGTCVNRWNMYLCECPLRFGGKNCEQAMPHPQLFSGESVVSWSDLNIIISVPWYLGLMFRTRKEDSVLMEATSGGPTSFRLQILNNYLQFEVSHGPSDVESVMLSGLRVTDGEWHHLLIELKNVKEDSEMKHLVTMTLDYGMDQNKADIGGMLPGLTVRSVVVGGASEDKVSVRRGFRGCMQGVRMGGTPTNVATLNMNNALKVRVKDGCDVDDPCTSSPCPPNSRCHDAWEDYSCVCDKGYLGINCVDACHLNPCENMGACVRSPGSPQGYVCECGPSHYGPYCENKLDLPCPRGWWGNPVCGPCHCAVSKGFDPDCNKTNGQCQCKENYYKLLAQDTCLPCDCFPHGSHSRTCDMATGQCACKPGVIGRQCNRCDNPFAEVTTLGCEVIYNGCPKAFEAGIWWPQTKFGQPAAVPCPKGSVGNAVRHCSGEKGWLPPELFNCTTISFVDLRAMNEKLSRNETQVDGARALQLVRALRSATQHTGTLFGNDVRTAYQLLGHVLQHESWQQGFDLAATQDADFHEDVIHSGSALLAPATRAAWEQIQRSEGGTAQLLRRLEGYFSNVARNVRRTYLRPFVIVTANMILAVDIFDKFNFTGARVPRFDTIHEEFPRELESSVSFPADFFRPPEEKEGPLLRPAGRRTTPQTTRPGPGTEREAPISRRRRHPDDAGQFAVALVIIYRTLGQLLPERYDPDRRSLRLPHRPIINTPMVSTLVYSEGAPLPRPLERPVLVEFALLEVEERTKPVCVFWNHSLAVGGTGGWSARGCELLSRNRTHVACQCSHTASFAVLMDISRRENGEVLPLKIVTYAAVSLSLAALLVAFVLLSLVRMLRSNLHSIHKHLAVALFLSQLVFVIGINQTENPFLCTVVAILLHYIYMSTFAWTLVESLHVYRMLTEVRNIDTGPMRFYYVVGWGIPAIVTGLAVGLDPQGYGNPDFCWLSLQDTLIWSFAGPIGAVIIINTVTSVLSAKVSCQRKHHYYGKKGIVSLLRTAFLLLLLISATWLLGLLAVNRDALSFHYLFAIFSGLQGPFVLLFHCVLNQEVRKHLKGVLGGRKLHLEDSATTRATLLTRSLNCNTTFGDGPDMLRTDLGESTASLDSIVRDEGIQKLGVSSGLVRGSHGEPDASLMPRSCKDPPGHDSDSDSELSLDEQSSSYASSHSSDSEDDGVGAEEKWDPARGAVHSTPKGDAVANHVPAGWPDQSLAESDSEDPSGKPRLKVETKVSVELHREEQGSHRGEYPPDQESGGAARLASSQPPEQRKGILKNKVTYPPPLTLTEQTLKGRLREKLADCEQSPTSSRTSSLGSGGPDCAITVKSPGREPGRDHLNGVAMNVRTGSAQADGSDSEKP

SEQ ID NO: 3 - DNA, 2898 bp, Homo sapiens
  CDS (1)..(2898)
atggagtttgtgcgggcgctgtggctgggcctggcgctggcgctggggccggggtccgcggggggccaccctcagccgtgcggcgtcctggcgcgcctcgggggctccgtgcgcctgggcgccctcctgccccgcgcgcctctcgcccgcgcccgcgcccgcgccgccctggcccgggccgccctggcgccgcggctgccgcacaacctgagcttggagctggtggtcgccgcgccccccgcccgcgaccccgcctcgctgacccgcggcctgtgccaggcgctggtgcctccgggcgtggcggccctgctcgcctttcccgaggctcggcccgagctgctgcagctgcacttcctggcggcggccaccgagacccccgtgctcagcctgctgcggcgggaggcgcgcgcgcccctcggagccccgaacccattccacctgcagctgcactgggccagccccctggagacgctgctggatgtgctggtggcggtgctgcaggcgcacgcctgggaagacgtcggcctggccctgtgccgcactcaggaccccggcggcctggtggccctctggacaagccgggctggccggcccccacagctggtcctggacctaagccggcgggacacgggagatgcaggactgcgggcacgcctggccccgatggcggcgccagtggggggtgaagcaccggtacccgcggcggtcctcctcggctgtgacatcgcccgtgcccgtcgggtgctggaggccgtacctcccggcccccactggctgttggggacaccactgccgcccaaggccctgcccaccgcggggctgccaccagggctgctggcgctgggcgaggtggcacgacccccgctggaggccgccatccatgacattgtgcaactggtggcccgggcgctgggcagtgcggcccaggtgcagccgaagcgagccctcctccccgccccggtcaactgcggggacctgcagccggccgggcccgagtccccggggcgcttcttggcacggttcctggccaacacgtccttccagggccgcacgggccccgtgtgggtgacaggcagctccccagacgaagacgggcagtgcccagcggggcagctgtgcctggaccctggcaccaacgactcggccaccctggacgcactgttcgccgcgctggccaacggctcagcgccccgtgccctgcgcaagtgctgctacggctactgcattgacctgctggagcggctggcggaggacacgcccttcgacttcgagctgtacctcgtgggtgacggcaagtacggcgccctgcgggacggccgctggaccggcctggtcggggacctgctggccggccgggcccacatggcggtcaccagcttcagtatcaactccgcccgctcacaggtggtggacttcaccagccccttcttctccaccagcctgggcatcatggtgcgggcacgggacacggcctcacccatcggtgcctttatgtggcccctgcactggtccacgtggctgggcgtctttgcggccctgcacctcaccgcgctcttcctcaccgtgtacgagtggcgtagcccctacggcctcacgccacgtggccgcaaccgcagcaccgtcttctcctactcctcagccctcaacctgtgctacgccatcctcttcagacgcaccgtgtccagcaagacgcccaagtgccccacgggccgcctgctcatgaacctctgggccatcttctgcctgctggtgctgtccagctacacggccaacctggctgccgtcatggtcggggacaagaccttcgaggagctgtcggggatccacgaccccaagggcttccgcttcggcaccgtgtgggagagcagcgccgaggcgtacatcaagaagagcttccccgacatgcacgcacacatgcggcgccacagcgcgcccaccacgccccgcggcgtcgccatgctcacgagcgacccccccaagctcaacgccttcatcatggacaagtcgctcctggactacgaggtctccatcgacgccgactgcaaactgctgaccgtgggaaagcccttcgccattgagggctatgggatcggactgccccagaactcgccgctcacctccaacctgtccgagttcatcagccgctacaagtcctccggcttcatcgacctgctccacgacaagtggtacaagatggtgccttgcggcaagcgggtctttgcggttacagagaccctgcagatgagcatctaccacttcgcgggcctcttcgtgttgctgtgcctgggcctgggcagcgctctgctcagctcgctgggcgagcacgccttcttccgcctggcgctgccgcgcatccgcaaggggagcaggctgcagtactggctgcacaccagccagaaaatccaccgcgccctcaacacggagccaccagaggggtcgaaggaggagacggcagaggcggagcccagcggccccgaggtggagcagcagcagcagcagcaggaccagccaacggctccggagggctggaaacgggcgcgccgggccgtggacaaggagcgccgcgtgcgcttcctgctggagcccgccgtggttgtggcacccgaagcggacgcggaggcggaggctgcgccgcgagagggccccgtctggctgtgctcctacggccgcccgcccgccgcaaggcccacgggggccccccagcccggggagctgcaggagctggagcgccgcatcgaagtcgcgcgtgagcggctccgccaggccctggtgcggcgcggccagctcctggcacagctcggggacagcgcacgtcaccggcctcggcgcttgcttcaggccagagcggcccccgcggaggccccaccacactctggccgaccggggagccaggaatga

SEQ ID NO: 4 - protein, 965 aa, Homo sapiens
MEFVRALWLGLALALGPGSAGGHPQPCGVLARLGGSVRLGALLPRAPLARARARAALARAALAPRLPHNLSLELVVAAPPARDPASLTRGLCQALVPPGVAALLAFPEARPELLQLHFLAAATETPVLSLLRREARAPLGAPNPFHLQLHWASPLETLLDVLVAVLQAHAWEDVGLALCRTQDPGGLVALWTSRAGRPPQLVLDLSRRDTGDAGLRARLAPMAAPVGGEAPVPAAVLLGCDIARARRVLEAVPPGPHWLLGTPLPPKALPTAGLPPGLLALGEVARPPLEAAIHDIVQLVARALGSAAQVQPKRALLPAPVNCGDLQPAGPESPGRFLARFLANTSFQGRTGPVWVTGSSPDEDGQCPAGQLCLDPGTNDSATLDALFAALANGSAPRALRKCCYGYCIDLLERLAEDTPFDFELYLVGDGKYGALRDGRWTGLVGDLLAGRAHMAVTSFSINSARSQVVDFTSPFFSTSLGIMVRARDTASPIGAFMWPLHWSTWLGVFAALHLTALFLTVYEWRSPYGLTPRGRNRSTVFSYSSALNLCYAILFRRTVSSKTPKCPTGRLLMNLWAIFCLLVLSSYTANLAAVMVGDKTFEELSGIHDPKGFRFGTVWESSAEAYIKKSFPDMHAHMRRHSAPTTPRGVAMLTSDPPKLNAFIMDKSLLDYEVSIDADCKLLTVGKPFAIEGYGIGLPQNSPLTSNLSEFISRYKSSGFIDLLHDKWYKMVPCGKRVFAVTETLQMSIYHFAGLFVLLCLGLGSALLSSLGEHAFFRLALPRIRKGSRLQYWLHTSQKIHRALNTEPPEGSKEETAEAEPSGPEVEQQQQQQDQPTAPEGWKRARRAVDKERRVRFLLEPAVVVAPEADAEAEAAPREGPVWLCSYGRPPAARPTGAPQPGELQELERRIEVARERLRQALVRRGQLLAQLGDSARHRPRRLLQARAAPAEAPPHSGRPGSQE

SEQ ID NO: 5 - DNA, 2916 bp, Homo sapiens
  CDS (1)..(2916)
atggagtttgtgcgggcgctgtggctgggcctggcgctggcgctggggccggggtccgcggggggccaccctcagccgtgcggcgtcctggcgcgcctcgggggctccgtgcgcctgggcgccctcctgccccgcgcgcctctcgcccgcgcccgcgcccgcgccgccctggcccgggccgccctggcgccgcggctgccgcacaacctgagcttggagctggtggtcgccgcgccccccgcccgcgaccccgcctcgctgacccgcggcctgtgccaggcgctggtgcctccgggcgtggcggccctgctcgcctttcccgaggctcggcccgagctgctgcagctgcacttcctggcggcggccaccgagacccccgtgctcagcctgctgcggcgggaggcgcgcgcgcccctcggagccccgaacccattccacctgcagctgcactgggccagccccctggagacgctgctggatgtgctggtggcggtgctgcaggcgcacgcctgggaagacgtcggcctggccctgtgccgcactcaggaccccggcggcctggtggccctctggacaagccgggctggccggcccccacagctggtcctggacctaagccggcgggacacgggagatgcaggactgcgggcacgcctggccccgatggcggcgccagtggggggtgaagcaccggtacccgcggcggtcctcctcggctgtgacatcgcccgtgcccgtcgggtgctggaggccgtacctcccggcccccactggctgttggggacaccactgccgcccaaggccctgcccaccgcggggctgccaccagggctgctggcgctgggcgaggtggcacgacccccgctggaggccgccatccatgacattgtgcaactggtggcccgggcgctgggcagtgcggcccaggtgcagccgaagcgagccctcctccccgccccggtcaactgcggggacctgcagccggccgggcccgagtccccggggcgcttcttggcacggttcctggccaacacgtccttccagggccgcacgggccccgtgtgggtgacaggcagctccccagacgaagacgggcagtgcccagcggggcagctgtgcctggaccctggcaccaacgactcggccaccctggacgcactgttcgccgcgctggccaacggctcagcgccccgtgccctgcgcaagtgctgctacggctactgcattgacctgctggagcggctggcggaggacacgcccttcgacttcgagctgtacctcgtgggtgacggcaagtacggcgccctgcgggacggccgctggaccggcctggtcggggacctgctggccggccgggcccacatggcggtcaccagcttcagtatcaactccgcccgctcacaggtggtggacttcaccagccccttcttctccaccagcctgggcatcatggtgcgggcacgggacacggcctcacccatcggtgcctttatgtggcccctgcactggtccacgtggctgggcgtctttgcggccctgcacctcaccgcgctcttcctcaccgtgtacgagtggcgtagcccctacggcctcacgccacgtggccgcaaccgcagcaccgtcttctcctactcctcagccctcaacctgtgctacgccatcctcttcagacgcaccgtgtccagcaagacgcccaagtgccccacgggccgcctgctcatgaacctctgggccatcttctgcctgctggtgctgtccagctacacggccaacctggctgccgtcatggtcggggacaagaccttcgaggagctgtcggggatccacgaccccaagctgcaccacccggcgcagggcttccgcttcggcaccgtgtgggagagcagcgccgaggcgtacatcaagaagagcttccccgacatgcacgcacacatgcggcgccacagcgcgcccaccacgccccgcggcgtcgccatgctcacgagcgacccccccaagctcaacgccttcatcatggacaagtcgctcctggactacgaggtctccatcgacgccgactgcaaactgctgaccgtgggaaagcccttcgccattgagggctatgggatcggactgccccagaactcgccgctcacctccaacctgtccgagttcatcagccgctacaagtcctccggcttcatcgacctgctccacgacaagtggtacaagatggtgccttgcggcaagcgggtctttgcggttacagagaccctgcagatgagcatctaccacttcgcgggcctcttcgtgttgctgtgcctgggcctgggcagcgctctgctcagctcgctgggcgagcacgccttcttccgcctggcgctgccgcgcatccgcaaggggagcaggctgcagtactggctgcacaccagccagaaaatccaccgcgccctcaacacggagccaccagaggggtcgaaggaggagacggcagaggcggagcccagcggccccgaggtggagcagcagcagcagcagcaggaccagccaacggctccggagggctggaaacgggcgcgccgggccgtggacaaggagcgccgcgtgcgcttcctgctggagcccgccgtggttgtggcacccgaagcggacgcggaggcggaggctgcgccgcgagagggccccgtctggctgtgctcctacggccgcccgcccgccgcaaggcccacgggggccccccagcccggggagctgcaggagctggagcgccgcatcgaagtcgcgcgtgagcggctccgccaggccctggtgcggcgcggccagctcctggcacagctcggggacagcgcacgtcaccggcctcggcgcttgcttcaggccagagcggcccccgcggaggccccaccacactctggccgaccggggagccaggaatga

SEQ ID NO: 6 - protein, 971 aa, Homo sapiens
MEFVRALWLGLALALGPGSAGGHPQPCGVLARLGGSVRLGALLPRAPLARARARAALARAALAPRLPHNLSLELVVAAPPARDPASLTRGLCQALVPPGVAALLAFPEARPELLQLHFLAAATETPVLSLLRREARAPLGAPNPFHLQLHWASPLETLLDVLVAVLQAHAWEDVGLALCRTQDPGGLVALWTSRAGRPPQLVLDLSRRDTGDAGLRARLAPMAAPVGGEAPVPAAVLLGCDIARARRVLEAVPPGPHWLLGTPLPPKALPTAGLPPGLLALGEVARPPLEAAIHDIVQLVARALGSAAQVQPKRALLPAPVNCGDLQPAGPESPGRFLARFLANTSFQGRTGPVWVTGSSPDEDGQCPAGQLCLDPGTNDSATLDALFAALANGSAPRALRKCCYGYCIDLLERLAEDTPFDFELYLVGDGKYGALRDGRWTGLVGDLLAGRAHMAVTSFSINSARSQVVDFTSPFFSTSLGIMVRARDTASPIGAFMWPLHWSTWLGVFAALHLTALFLTVYEWRSPYGLTPRGRNRSTVFSYSSALNLCYAILFRRTVSSKTPKCPTGRLLMNLWAIFCLLVLSSYTANLAAVMVGDKTFEELSGIHDPKLHHPAQGFRFGTVWESSAEAYIKKSFPDMHAHMRRHSAPTTPRGVAMLTSDPPKLNAFIMDKSLLDYEVSIDADCKLLTVGKPFAIEGYGIGLPQNSPLTSNLSEFISRYKSSGFIDLLHDKWYKMVPCGKRVFAVTETLQMSIYHFAGLFVLLCLGLGSALLSSLGEHAFFRLALPRIRKGSRLQYWLHTSQKIHRALNTEPPEGSKEETAEAEPSGPEVEQQQQQQDQPTAPEGWKRARRAVDKERRVRFLLEPAVVVAPEADAEAEAAPREGPVWLCSYGRPPAARPTGAPQPGELQELERRIEVARERLRQALVRRGQLLAQLGDSARHRPRRLLQARAAPAEAPPHSGRPGSQE

SEQ ID NO: 7 - DNA, 3132 bp, Homo sapiens
  CDS (1)..(3129)
atggagtttgtgcgggcgctgtggctgggcctggcgctggcgctggggccggggtccgcggggggccaccctcagccgtgcggcgtcctggcgcgcctcgggggctccgtgcgcctgggcgccctcctgccccgcgcgcctctcgcccgcgcccgcgcccgcgccgccctggcccgggccgccctggcgccgcggctgccgcacaacctgagcttggagctggtggtcgccgcgccccccgcccgcgaccccgcctcgctgacccgcggcctgtgccaggcgctggtgcctccgggcgtggcggccctgctcgcctttcccgaggctcggcccgagctgctgcagctgcacttcctggcggcggccaccgagacccccgtgctcagcctgctgcggcgggaggcgcgcgcgcccctcggagccccgaacccattccacctgcagctgcactgggccagccccctggagacgctgctggatgtgctggtggcggtgctgcaggcgcacgcctgggaagacgtcggcctggccctgtgccgcactcaggaccccggcggcctggtggccctctggacaagccgggctggccggcccccacagctggtcctggacctaagccggcgggacacgggagatgcaggactgcgggcacgcctggccccgatggcggcgccagtggggggtgaagcaccggtacccgcggcggtcctcctcggctgtgacatcgcccgtgcccgtcgggtgctggaggccgtacctcccggcccccactggctgttggggacaccactgccgcccaaggccctgcccaccgcggggctgccaccagggctgctggcgctgggcgaggtggcacgacccccgctggaggccgccatccatgacattgtgcaactggtggcccgggcgctgggcagtgcggcccaggtgcagccgaagcgagccctcctccccgccccggtcaactgcggggacctgcagccggccgggcccgagtccccggggcgcttcttggcacggttcctggccaacacgtccttccagggccgcacgggccccgtgtgggtgacaggcagctcccaggtacacatgtctcggcactttaaggtgtggagccttcgccgggacccacggggcgccccggcctgggccacggtgggcagctggcgggacggccagctggacttggaaccgggaggtgcctctgcacggcccccgcccccacagggtgcccaggtctggcccaagctgcgtgtggtaacgctgttggaacacccatttgtgtttgcccgtgatccagacgaagacgggcagtgcccagcggggcagctgtgcctggaccctggcaccaacgactcggccaccctggacgcactgttcgccgcgctggccaacggctcagcgccccgtgccctgcgcaagtgctgctacggctactgcattgacctgctggagcggctggcggaggacacgcccttcgacttcgagctgtacctcgtgggtgacggcaagtacggcgccctgcgggacggccgctggaccggcctggtcggggacctgctggccggccgggcccacatggcggtcaccagcttcagtatcaactccgcccgctcacaggtggtggacttcaccagccccttcttctccaccagcctgggcatcatggtgcgggcacgggacacggcctcacccatcggtgcctttatgtggcccctgcactggtccacgtggctgggcgtctttgcggccctgcacctcaccgcgctcttcctcaccgtgtacgagtggcgtagcccctacggcctcacgccacgtggccgcaaccgcagcaccgtcttctcctactcctcagccctcaacctgtgctacgccatcctcttcagacgcaccgtgtccagcaagacgcccaagtgccccacgggccgcctgctcatgaacctctgggccatcttctgcctgctggtgctgtccagctacacggccaacctggctgccgtcatggtcggggacaagaccttcgaggagctgtcggggatccacgaccccaagctgcaccacccggcgcagggcttccgcttcggcaccgtgtgggagagcagcgccgaggcgtacatcaagaagagcttccccgacatgcacgcacacatgcggcgccacagcgcgcccaccacgccccgcggcgtcgccatgctcacgagcgacccccccaagctcaacgccttcatcatggacaagtcgctcctggactacgaggtctccatcgacgccgactgcaaactgctgaccgtgggaaagcccttcgccattgagggctatgggatcggactgccccagaactcgccgctcacctccaacctgtccgagttcatcagccgctacaagtcctccggcttcatcgacctgctccacgacaagtggtacaagatggtgccttgcggcaagcgggtctttgcggttacagagaccctgcagatgagcatctaccacttcgcgggcctcttcgtgttgctgtgcctgggcctgggcagcgctctgctcagctcgctgggcgagcacgccttcttccgcctggcgctgccgcgcatccgcaaggggagcaggctgcagtactggctgcacaccagccagaaaatccaccgcgccctcaacacggagccaccagaggggtcgaaggaggagacggcagaggcggagcccagcggccccgaggtggagcagcagcagcagcagcaggaccagccaacggctccggagggctggaaacgggcgcgccgggccgtggacaaggagcgccgcgtgcgcttcctgctggagcccgccgtggttgtggcacccgaagcggacgcggaggcggaggctgcgccgcgagagggccccgtctggctgtgctcctacggccgcccgcccgccgcaaggcccacgggggccccccagcccggggagctgcaggagctggagcgccgcatcgaagtcgcgcgtgagcggctccgccaggccctggtgcggcgcggccagctcctggcacagctcggggacagcgcacgtcaccggcctcggcgcttgcttcaggccagagcggcccccgcggaggccccaccacactctggccgaccggggagccaggaatga

SEQ ID NO: 8 - protein, 1043 aa, Homo sapiens
MEFVRALWLGLALALGPGSAGGHPQPCGVLARLGGSVRLGALLPRAPLARARARAALARAALAPRLPHNLSLELVVAAPPARDPASLTRGLCQALVPPGVAALLAFPEARPELLQLHFLAAATETPVLSLLRREARAPLGAPNPFHLQLHWASPLETLLDVLVAVLQAHAWEDVGLALCRTQDPGGLVALWTSRAGRPPQLVLDLSRRDTGDAGLRARLAPMAAPVGGEAPVPAAVLLGCDIARARRVLEAVPPGPHWLLGTPLPPKALPTAGLPPGLLALGEVARPPLEAAIHDIVQLVARALGSAAQVQPKRALLPAPVNCGDLQPAGPESPGRFLARFLANTSFQGRTGPVWVTGSSQVHMSRHFKVWSLRRDPRGAPAWATVGSWRDGQLDLEPGGASARPPPPQGAQVWPKLRVVTLLEHPFVFARDPDEDGQCPAGQLCLDPGTNDSATLDALFAALANGSAPRALRKCCYGYCIDLLERLAEDTPFDFELYLVGDGKYGALRDGRWTGLVGDLLAGRAHMAVTSFSINSARSQVVDFTSPFFSTSLGIMVRARDTASPIGAFMWPLHWSTWLGVFAALHLTALFLTVYEWRSPYGLTPRGRNRSTVFSYSSALNLCYAILFRRTVSSKTPKCPTGRLLMNLWAIFCLLVLSSYTANLAAVMVGDKTFEELSGIHDPKLHHPAQGFRFGTVWESSAEAYIKKSFPDMHAHMRRHSAPTTPRGVAMLTSDPPKLNAFIMDKSLLDYEVSIDADCKLLTVGKPFAIEGYGIGLPQNSPLTSNLSEFISRYKSSGFIDLLHDKWYKMVPCGKRVFAVTETLQMSIYHFAGLFVLLCLGLGSALLSSLGEHAFFRLALPRIRKGSRLQYWLHTSQKIHRALNTEPPEGSKEETAEAEPSGPEVEQQQQQQDQPTAPEGWKRARRAVDKERRVRFLLEPAVVVAPEADAEAEAAPREGPVWLCSYGRPPAARPTGAPQPGELQELERRIEVARERLRQALVRRGQLLAQLGDSARHRPRRLLQARAAPAEAPPHSGRPGSQE

SEQ ID NO: 9 - DNA, 1110 bp, Homo sapiens
  CDS (163)..(990)
acgcgttactcctaccaggttgtagcatgcatctttttgagagagcagctgggatcgagtatactcttgacttaaatatgtttgtttataaagacaaatggagaaatcaatttttttccctgaattcttaggagcactttagtgaataaagaacctgacagtatgctggcccacatgtttaaggacaaaggtgtctggggaaataagcaagatcatagaggagctttcttaattgaccgaagtcctgagtacttcgaacccattttgaactacttgcgtcatggacagctcattgtaaatgatggcattaatttattgggtgtgttagaagaagcaagattttttggtattgactcattgattgaacacctagaagtggcaataaagaattctcaaccaccggaggatcattcaccaatatcccgaaaggaatttgtccgatttttgctagcaactccaaccaagtcagaactgcgatgccagggtttgaacttcagtggtgctgatctttctcgtttggaccttcgatacattaacttcaaaatggccaatttaagccgctgtaatcttgcacatgcaaatctttgctgtgcaaatcttgaacgagctgatctctctggatcagtgcttgactgtgcgaatctccagggagtcaagatgctctgttctaatgcagaaggagcatccctgaaactgtgtaattttgaggatccttctggtcttaaagccaatttagaaggtgctaatctgaaaggtgtggatatggaaggaagtcagatgacaggaattaacctgagagtggctaccttaaaaaatgcaaagttgaagaactgtaacctcagaggagcaactctggcaggaactgatttagagaattgtgatctgtctgggtgtgatcttcaagaagccaacctgagagggtccaacgtgaagggagctatatttgaagagatgctgacaccactacacatgtcacaaagtgtcagatgagaattttaggggctggaggaagatgtaaaagatgaaaatgttttccttatcacttttctttctccacccactcagttgtctagaagaaataacactgtaaggaaatttaaaaaaaaaaaa

SEQ ID NO: 10 - protein, 275 aa, Homo sapiens
MLAHMFKDKGVWGNKQDHRGAFLIDRSPEYFEPILNYLRHGQLIVNDGINLLGVLEEARFFGIDSLIEHLEVAIKNSQPPEDHSPISRKEFVRFLLATPTKSELRCQGLNFSGADLSRLDLRYINFKMANLSRCNLAHANLCCANLERADLSGSVLDCANLQGVKMLCSNAEGASLKLCNFEDPSGLKANLEGANLKGVDMEGSQMTGINLRVATLKNAKLKNCNLRGATLAGTDLENCDLSGCDLQEANLRGSNVKGAIFEEMLTPLHMSQSVR

SEQ ID NO: 11 - DNA, 926 bp, Homo sapiens
  CDS (33)..(887)
tttccagggttctagcctgttcatctagccccatgatggctgtggacatcgagtacagatacaactgcatggctccttccttgcgccaagagaggtttgcctttaagatctcaccaaagcccagcaaaccactgaggccttgtattcagctgagcagcaagaatgaagccagtggaatggtggccccggctgtccaggagaagaaggtgaaaaagcgggtgtccttcgcagacaaccaggggctggccctgacaatggtcaaagtgttctcggaattcgatgacccgctagatatgccattcaacatcaccgagctcctagacaacattgtgagcttgacgacagcagagagcgagagctttgttctggatttttcccagccctctgcagattacttagactttagaaatcgacttcaggccgaccacgtctgccttgagaactgtgtgctcaaggacaaggccattgcaggcactgtgaaggttcagaacctcgcatttgagaagaccgtgaaaataaggatgacgttcgacacctggaagagctacacagactttccttgtcagtacgtgaaggacacttatgccggttcagacagggacacgttctccttcgacatcagcttgcccgagaagattcagtcttatgaaagaatggagtttgctgtgtactacgagtgcaatggacagacgtactgggacagcaacagaggcaagaactataggatcatccgggctgagttaaaatctacccagggaatgaccaagccccacagtggaccggatttgggaatatcctttgaccagttcggaagccctcggtgttcctatggtctgtttccagagtggccaagttacttaggatatgaaaagctagggccctactactagtgactgcaggtgacagggcgtggcggagctgccaca

SEQ ID NO: 12 - protein, 285 aa, Homo sapiens
MMAVDIEYRYNCMAPSLRQERFAFKISPKPSKPLRPCIQLSSKNEASGMVAPAVQEKKVKKRVSFADNQGLALTMVKVFSEFDDPLDMPFNITELLDNIVSLTTAESESFVLDFSQPSADYLDFRNRLQADHVCLENCVLKDKAIAGTVKVQNLAFEKTVKIRMTFDTWKSYTDFPCQYVKDTYAGSDRDTFSFDISLPEKIQSYERMEFAVYYECNGQTYWDSNRGKNYRIIRAELKSTQGMTKPHSGPDLGISFDQFGSPRCSYGLFPEWPSYLGYEKLGPYY

SEQ ID NO: 13 - DNA, 551 bp, Homo sapiens
  CDS (38)..(505)
ctgtctcctgcattctcctgaaaccttcatccacacaatgcctcccaacctcactggctactaccgctttgtctcgcagaagaacatggaggactacctgcaagccctaaacatcagcttggctgtgcggaagatcgcgctgctgctgaagccggacaaggagatcgaacaccagggcaaccacatgacggtgaggacgctcagcaccttccgaaactacactgtgcagtttgatgtgggagtggagtttgaggaggacctcaggagcgtggacggacgaaaatgccagatctcattcgtcggttcggatccaagccagttctgtggtcagcaaggctcccctctgggcaggccccctggtcagagggagtttgtatcctcagggaggagtttgcggctgaccttccgcacacagccttcctcggagaacaagactgcccacctccacaagggcttcctggccctctaccaaaccgtggccttaagtggaagcttgagtgacagctgaggctggggactcagggacacctgggctggatcccagccctgcc

SEQ ID NO: 14 - protein, 156 aa, Homo sapiens
MPPNLTGYYRFVSQKNMEDYLQALNISLAVRKIALLLKPDKEIEHQGNHMTVRTLSTFRNYTVQFDVGVEFEEDLRSVDGRKCQISFVGSDPSQFCGQQGSPLGRPPGQREFVSSGRSLRLTFRTQPSSENKTAHLHKGFLALYQTVALSGSLSDS

SEQ ID NO: 15 - DNA, 817 bp, Homo sapiens
  CDS (38)..(442)
ctgtctcctgcattctcctgaaaccttcatccacacaatgcctcccaacctcactggctactaccgctttgtctcgcagaagaacatggaggactacctgcaagccctaaacatcagcttggctgtgcggaagatcgcgctgctgctgaagccggacaaggagatcgaacaccagggcaaccacatgacggtgaggacgctcagcaccttccgaaactacactgtgcagtttgatgtgggagtggagtttgaggaggacctcaggagcgtggacggacgaaaatgccagaccatagtaacctgggaggaggagcacctggtgtgtgtgcagaaaggggaggtccccaaccggggctggagacactggctggagggagagttgctgtatctggaactgactgcaagggatgcagtgtgcgagcaggtcttcaggaaggtcagatagccggagaggagccaagatccctccagacagcaccagctcacagacgctcttgttgtgcccccttcaagcccagattgtgccagatctcattcgtcggttcggatccaagccagttctgtggtcagcaaggctcccctctgggcaggccccctggtcagagggagtttgtatcctcagggaggagtttgcggctgaccttccgcacacagccttcctcggagaacaagactgcccacctccacaagggcttcctggccctctaccaaaccgtgggtgagtgtccctcctgggggtgcagggagggagcctctgttcccagccatgaccctggtatcttcaagccttaagtggaagcttgagtgacagctgagg

SEQ ID NO: 16 - protein, 135 aa, Homo sapiens [sequence omitted]

SEQ ID NO: 17 - DNA, 21 bp, Artificial Sequence
  Ag765 Forward Primer
ccaacgtgaagggagctatat

SEQ ID NO: 18 - DNA, 26 bp, Artificial Sequence
  Ag765 Probe Primer
tgctgacaccactacacatgtcacaa

SEQ ID NO: 19 - DNA, 21 bp, Artificial Sequence
  Ag765 Reverse Primer
ccagcccctaaaattctcatc

SEQ ID NO: 20 - DNA, 22 bp, Artificial Sequence
  Ag1387 Forward Primer
ctgaaaccttcatccacacaat

SEQ ID NO: 21 - DNA, 26 bp, Artificial Sequence
  Ag1387 Probe Primer
tcactggctactaccgctttgtctcg

SEQ ID NO: 22 - DNA, 22 bp, Artificial Sequence
  Ag1387 Reverse Primer
gcaggtagtcctccatgttctt

SEQ ID NO: 23 - DNA, 218 bp, Homo sapiens
ctgaagtcacaggccctgcctctggcttttgcaggagaattactacaagctcctagcccaggacacctgtctgccctgcgactgcttcccccatggctcccacagccgcacttgcgacatggccaccgggcagtgtgcctgcaagcccggcgtcatcggccgccagtgcaaccgctgcgacaacccgtttgccgaggtcaccacgctcggctgtgaag

SEQ ID NO: 24 - DNA, 220 bp, Artificial Sequence
  Consensus Sequence
ctgnantnanannnncngccnntgncnntngcanggagaattactacaagctcctagcccaggacacctgtctgccctgcgactgcttcccccatggctcccacagccgcacttgcgacatggccaccgggcagtgtgcctgcaagcccggcgtcatcggccgccagtgcaaccgctgcgnacaacccgtttgccgaggtcaccacgctcggctgtgaag

SEQ ID NO: 25 - protein, 3034 aa, Mus musculus
MAPSSPRVLPALVLLAAAALPALELGAAAWELRVPGGARAFALGPGWSYRLDTTRTPRELLDVSREGPAAGRRLGLGAGTLGCARLAGRLLPLQVRLVARGAPTAPSLVLRARAYGARCGVRLLRRSARGAELRSPAVRSVPGLGDALCFPAAGGGAASLTSVLEAITNFPACSCPPVAGTGCRRGPICLRPGGSAELRLVCALGRAAGAVWVELVIQATSGTPSESPSVSPSLLNLSQPRAGVVRRSRRGTGSSTSPQFPLPSYQVSVPENEPAGTAVIELRAHDPDEGDAGRLSYQMEALFDERSNGYFLIDAATGAVTTARSLDRETKDTHVLKVSAVDHGSPRRSAATYLTVTVSDTNDHSPVFEQSEYRERIRENLEVGYEVLTIRATDGDAPSNANMRYRLLEGAGGVFEIDARSGVVRTRAVVDREEAAEYQLLVEANDQGRNPGPLSASATVHIVVEDENDNYPQFSEKRYVVQVPEDVAVNTAVLRVQATDRDQGQNAAIHYSIVSGNLKGQFYLHSLSGSLDVINPLDFEAIREYTLRIKAQDGGRPPLINSSGLVSVQVLDVNDNAPIFVSSPFQAAVLENVPLGHSVLHIQAVDADAGENARLQYRLVDTASTIVGGSSVDSENPASAPDFPFQIHNSSGWITVCAELDREEVEHYSFGVEAVDHGSPAMSSSASVSITVLDVNDNDPMFTQPVYELRLNEDAAVGSSVLTLRARDRDANSVITYQLTGGNTRNRFALSSQSGGGLITLALPLDYKQERQYVLAVTASDGTRSHTAQVFINVTDANTHRPVFQSSHYTVSVSEDRPVGTSIATISATDEDTGENARITYVLEDPVPQFRIDPDTGTIYTMTELDYEDQAAYTLAITAQDNGIPQKSDTTSLEILILDANDNAPRFLRDFYQGSVFEDAPPSTSVLQVSATDRDSGPNGRLLYTFQGGDDGDGDFYIEPTSGVIRTQRRLDRENVAVYNLWALAVDRGSPNPLSASVGIQVSVLDINDNPPVFEKDELELFVEENSPVGSVVARIRANDPDEGPNAQIIYQIVEGNVPEVFQLDLLSGDLRALVELDFEVRRDYMLVVQATSAPLVSRATVHIRLLDQNDNPPELPDFQILFNNYVTNKSNSFPSGVIGRIPAHDPDLSDSLNYTFLQGNELSLLLLDPATGELQLSRDLDNNRPLEALMEVSVSDGIHSVTALCTLRVTIITDDMLTNSITVRLENMSQEKFLSPLLSLFVEGVATVLSTTKDDIFVFNIQNDTDVSSNILNVTFSALLPGGTRGRFFPSEDLQEQIYLNRTLLTTISAQRVLPFDDNICLREPCENYMKCVSVLRFDSSAPFISSTTVLFRPIHPITGLRCRCPPGFTGDYCETEIDLCYSNPCGANGRCRSREGGYTCECFEDFTGEHCQVNVRSGRCASGVCKNGGTCVNLLIGGFHCVCPPGEYEHPYCEVSTRSFPPQSFVTFRGLRQRFHFTVSLAFATQDRNALLLYNGRFNEKHDFIALEIVEEQLQLTFSAGETTTTVTPQVPGGVSDGRWHSVLVQYYNKPNIGHLGLPHGPSGEKVAVVTVDDCDAAVAVHFGSYVGNYSCAAQGTQSGSKKSLDLTGPLLLGGVPNLPEDFPVHSRQFVGCMRNLSIDGRIVDMAAFIANNGTRAGCASQRNFCDGTSCQNGGTCVNRWNTYLCECPLRFGGKNCEQAMPHPQRFTGESVVLWSDLDITISVPWYLGLMFRTRKEDGVLMEATAGTSSRLHLQILNSYIRFEVSYGPSDVASMQLSKSRITDGGWHHLLIELRSAKEGKDIKYLAVMTLDYGMDQSTVQIGNQLPGLKMRTIVIGGVTEDKVSVRHGFRGCMQGVRMGETSTNIATLNMNDALKVRVKDGCDVEDPCASSPCPPHRPCRDTWDSYSCICDRGYFGKKCVDACLLNPCKHVAACVRSPNTPRGYSCECGPGHYGQYCENKVDLPCPKGWWGNPVCGPCHCAVSQGFDPDCNKTNGQCQCKENYYKPPAQDACLPCDCFPHGSHSRACDMDTGQCACKPGVIGRQCNRCDNPFAEVTSLGCEVIYNGCPRAFEAGIWWPQTKFGQPAAVPCPKGSVGNAVRHCSGEKGWLPPELFNCTSGSFVDLKALNEKLNRNETRMDGNRSLRLAKALRNATQGNSTLFGNDVRTAYQLLARILQHESRQQGFDLAATREANFHEDVVHTGSALLAPATEASWEQIQRSEAGAAQLLRHFEAYFSNVARNVKRTYLRPFVIVTANMILAVDIFDKLNFTGAQVPRFEDIQEELPRELESSVSFPADTFKPPEKKEGPVVRLTNRRTTPLTAQPEPRAERETSSSRRRRHPDEPGQFAVALVVIYRTLGQLLPEHYDPDHRSLRLPNRPVINTPVVSAMVYSEGTPLPSSLQRPILVEFSLLETEERSKPVCVFWNHSLDTGGTGGWSAKGCELLSRNRTHVTCQCSHSASCAVLMDISRREHGEVLPLKIITYAALSLSLVALLVAFVLLSLVRTLRSNLHSIHKNLIAALFFSQLIFMVGINQTENPFLCTVVAILLHYVSMGTFAWTLVENLHVYRMLTEVRNIDTGPMRFYHVVGWGIPAIVTGLAVGLDPQGYGNPDFCWLSLQDTLIWSFAGPVGTVIIINTVIFVLSAKVSCQRKHHYYERKGVVSMLRTAFLLLLLVTATWLLGLLAVNSDTLSFHYLFAAFSCLQGIFVLLFHCVAHREVRKHLRAVLAGKKLQLDDSATTRATLLTRSLNCNNTYSEGPDMLRTALGESTASLDSTTRDEGVQKLSVSSGPARGNHGEPDTSFIPRNSKKAHGPDSDSDSELSLDEHSSSYASSHTSDSEDDGGEAEDKWNPAGGPAHSTPKADALANHVPAGWPDESLAGSDSEELDTEPHLKVETKVSVELHRQAQGNHCGDRPSDPESGVLAKPVAVLSSQPQEQRKGILKNKVTYPPPLPEQPLKSRLREKLADCEQSPTSSRTSSLGSGDGVHATDCVITIKTPRREPGREHLNGVAMNVRTGSAQANGSDSEKP

SEQ ID NO: 26 - protein, 120 aa, Artificial Sequence
  Consensus Sequence
YXGXXCVDACXLNPCXXXXACVRSPXXPXGYXCECGPXHYGXYCENKXDLPCPXGWWGNPVCGPCHCAVSXGFDPDCNKTNGQCQCKENYYKXXAQDXCLPCDCFPHGSHSRXCDMXTGQ

SEQ ID NO: 27 - protein, 1713 aa, Artificial Sequence
  Consensus Sequence
MAPXXPXVLPXLXLAAAAXLPAXXLXAAAWEXRVPGGXRAFALXPGXXYXXXXXTPRXREXXXXGRXXGAGXXXXXXXAGRXLPLQVRLVARXAPTAXSXXLRARXXXXXCXGXRXXRXXXXGAXLXXXXXXXXXXGALCFPXXGGXAAXXXSXLXAXTXXPACXCPPXCXXXPICLXPGGSXXLRLXCALXRAAGAVXVXLXXXAXTXGTPSXSPSXSPXLXXNLXXXRAGXXRRXRRGTXXXXSXXFPXPXYQVXXXENEPAGTXXXXLXAHXEGXXXRXSYXMEXLFDERSXGYFXIDXATGAVXTXXXLDRETKXTHVLXVXAVDXXXPXRSAXTYXTVVDTNDHSPVFEQSEYRERXRENLEVGYEVLTIRAXDXDXPXNANXRYRXLXGAXXVFXXXXXSGVVTRAVXDREEAAEYQLLVEANDQGRNPGPLSAATVXIXVEDENDNYPQFSEXYVVQVPEDVXXNTAVLRVQATDRDQGQNAAIHYSIXSGNXXGQFYLHSLSGXLDVINPLXDFEXXXXYXLIKAQDGGRPPLINSSGVSVQVLDVNDNXPIFVSSPFQAVLENVPLGXXVXHIQAVDADXGENARLXYRLVDTASTXXGGXSXXXXNPAXPDFPFQIHNSSGWITVCAELDREEVEHYSFGVEAVDHGSPXMSSSSVSITVLDVNDNDPXFTQPYELRLNEDAAVXGSSVLTLXARDRDANSVITYQLTGGNTRNRFALSSQGGGLITLALPLDYKQEXQYVLAVXTASDGTRSHTAXVINVTDANTHRPVFQSSHYTVSVSEDRPVGTSIATSAXDEDTGENAXRITYVXXDPVPQFRIDPDXGTXYTMXELDYEXQXAYTLXIXAQDNGIPQKSDTTXLEILILDANDNAPXFLXDFYQGSXFEDAPPSTSXLQVSATDRDSGPNGRLLYTFQGGDDGDGDFYIEPTSGVIRTQRRLDRENVAVYNLWALAVDRGSPXPLSASVXIQVXXLDINDNXPXFEKDELELFVEENXPVGSVVAXIRANDPDEGPNAQIXYQIVEGXXXXXFQLDLLXGDLRAXVELDFEVRRXYXLVVQATSAPLVSRATVHIXLXDQNDNPPXLPDFQILFNNYVTNKSNSFPXGXVIGIPAHDPDXSDSLNYTFXQGNELXLLLLDPATGELQLSRDLDNNRPLEALMEVSVSDXGIHSVTACTLRVTIITDDMLTNSITVRLENMSQEKFLSPLLXLFVEGVAXVLSTTKDDXFVFNXQNDTDVSSNILNVTFSALLPGGXRGXFFPSEDLQEQIYLNRTLLTTISXQRVLPFDDNICLREPCENYMKCVSVLRFDSSAPFXSSTTVLFRPIHPIXGLRCRCPPGFTGDYCETEIDLCYSPCGANGRCRSREGGYTCECFEDFTGEHCXVXXRSGRCAXGVCKNGGTCVNLLIGGFHCVCPPGEYEXPYCEVXTRSFPPQSFVTFRGLRQRFHFTXSLXFATQXRNXLLLYNGRFNEKHDFIALEIVXEQXQLTFSAGETTTTVXPXVPXGVSDGRWHSVXVQYYNKPNIGHLGLPHGPSGEKXAVVTVDDCDXXXAVXFGXXXGNYSCAAQGTQXGSKKSLDLTGPLLLGGVPNLPEDFPVHXRQFVGCMRNLSXDGXXVDMAXFIANNGTRXGCAXXRNFCDGXXCQNGGXTCVNRWNYLCECPLRFGGKNCEQAMPHPQXFXGESVVXWSDLXIXISVPWYLGLMFRTR

SEQ ID NO: 28 - protein, 458 aa, Artificial Sequence
  Consensus Sequence
VRNIDTGPMRFYXVVGWGIPAIVTGLAVGLDPQGYGNPDFCWLSLQDTLIWSFAGPXGXVIIINTVXXVLSAKVSCQRKHHYYXXKGXVSXLRTAFLLLLLXXATWLLGLLAVNXDXLSFHYLFAXFSXLQCXFVLLFHCVXXXEVRKHLXXVLXGXKLXLXDSATTRATLLTRSLNCNXTXXXGPDMLRTXLGESTASLDSXXRDEGXQKLXVSSGXRGXHGEPDXSXXPRXXKXXXGXDSDSDSELSLDEXSSSYASSHXSDSEDDGXXAEXKWXPAXGXXHSTPKXDAXANHVPAGWPDXSLAXSDSEXXXXXPXLKVETKVSVELHRXXQGXHXGXXPXDXESGXXAXXXXXXSSQPXEQRKGILKNKVTYPPPLXXXEQXLKXRLREKLADCEQSPTSSRTSSLGSGXXXXXXDCXITXKXPXREPGRXHLNGVAMNVRTGSAQAXGSDSEKP

SEQ ID NO: 29 - protein, 3313 aa, Rattus norvegicus
MARRPLWWGLPGPSTPLLLLLLFSLFPSSREEMGGGGDQGWDPGVATATGPRAQIGSGAVALCPESPGVWEDGDPGLGVREPVFMKLRVGRQNARNGRGAPEQPNREPVVQALGSREQEAGQGSGYLLCWHPEISSCGRTGHLRRGSLPLDALSPGDSDLRNSSPHPSELLAQPDSPRPVAFQRNGRRSIRKRVETFRCCGKLWEPGHKGQGERSATSTVDRGPLRRDCLPGSLGSGLGEDSAPRAVRTAPAPGSAPHESRTAPERMRSRGLFRRGFLFERPGPRPPGFPTGAEAKRILSTNQARSRRAANRHPQFPQYNYQTLVPENEAAGTAVLRVVAQDPDPGEAGRLVYSLAALMNSRSLELFSIDPQSGLIRTAAALDRESMERHYLRVTAQDHGSPRLSATTMVAVTVADRNDHAPVFEQAQYRETLRENVEEGYPILQLRATDGDAPPNANLRYRFVGSPAARTAAAAAFEIDPRSGLISTSGRVDREHMESYELVVEASDQGQEPGPRSATVRVHITVLDENDNAPQFSEKRYVAQVREDVRPHTVVLRVTATDKDKDANGLVHYNIISGNSRGHFAIDSLTGEIQVMAPLDFEAEREYALRIRAQDAGRPPLSNNTGLASIQVVDINDHSPIFVSTPFQVSVLENAPLGHSVIHIQAVDADHGENSRLEYSLTGVASDTPFVINSATGWVSVSGPLDRESVEHYFFGVEARDHGSPPLSASASVTVTVLDVNDNRPEFTMKEYHLRLNEDAAVGTSVVSVTAVDRDANSAISYQITGGNTRNRFAISTQGGMGLVTLALPLDYKQERYFKLVLTASDRALHDHCYVHINITDANTHRPVFQSAHYSVSMNEDRPVGSTVVVISASDDDVGENARITYLLEDNLPQFRIDADSGAITLQAPLDYEDQVTYTLAITARDNGIPQKADTTYVEVMVNDVNDNAPQFVASHYTGLVSEDAPPFTSVLQISATDRDAHANGRVQYTFQNGEDGDGDFTIEPTSGIVRTVRRLDREAVPVYELTAYAVDRGVPPLRTPVSIQVTVQDVNDNAPVFPAEEFEVRVKENSIVGSVVAQITAVDPDDGPNAHIMYQIVEGNIPELFQMDIFSGELTALIDLDYEARQEYVIVVQATSAPLVSRATVHVRLVDQNDNSPVLNNFQILFNNYVSNRSDTFPSGIIGRIPAYDPDVSDHLFYSFERGNELQLLVVNQTSGELRLSRKLDNNRPLVASMLVTVTDGLHSVTAQCVLRVVIITEELLANSLTVRLENMWQERFLSPLLGHFLEGVAAVLATPTEDVFIFNIQNDTDVGGTVLNVSFSALAPRGAGAGAAGPWFSSEELQEQLYVRRAALAARSLLDVLPFDDNVCLREPCENYMKCVSVLRFDSSAPFLASASTLFRPIQPIAGLRCRCPPGFTGDFCETELDLCYSNPCRNGGACARREGGYTCVCRPRFTGEDCELDTEAGRCVPGVCRNGGTCTNAPNGGFRCQCPAGGAFEGPRCEVAARSFPPSSFVMFRGLRQRFHLTLSLSFATVQPSGLLFYNGRLNEKHDFLALELVAGQVRLTYSTGESSTVVSPTVPGGLSDGQWHTVHLRYYNKPRTDALGGAQGPSKDKVAVLSVDDCNVAVALRFGAEIGNYSCAAAGVQTSSKKSLDLTGPLLLGGVPNLPENFPVSRKDFIGCMRDLHIDGRRVDMAAFVANNGTTAGCQAKSHFCASGPCKNGGLCSERWGGFSCDCPVGFGGKDCRLTMAHPYHFQGNGTLSWDFGNDMPVSVPWYLGLSFRTRATKGVLMQVQLGPHSVLLCKLDQGLLSVTLSRASGHAVHLLLDQMTVSDGRWHDLRLELQEEPGGRRGHHIFMVSLDFTLFQDTMAMGSELEGLKVKHLHVGGPPPSSKEEGPQGLVGCIQGVWTGFTPFGSSALPPPSHRINVEPGCTVTNPCASGPCPPHANCKDLWQTFSCTCWPGYYGPGCVDACLLNPCQNQGSCRHLQGGPHGYTCDCASGYFGQHCEHRMDQQCPRGWWGSPTCGPCNCDVHKGFDPNCNKTSGQCHCKEFHYRPRGSDSCLPCDCYPVGSTSRSCAPHSGQCPCRPGALGRQCNSCDSPFAEVTASGCRVLYDACPKSLRSGVWWPQTKFGVLATVPCPRGALGLRGTGAAVRLCDEDHGWLEPDFFNCTSPAFRELSLLLDGLELNKTALDTVEAKKLAQRLREVTGQTDHYFSQDVRVTARLLAYLLAFESHQQGFGLTATQDAHFNENLLWAGSALLAPETGDLWAALGQRAPGGSPGSAGLVRHLEEYAATLARNMDLTYLNPVGLVTPNIMLSIDRMEQPSSSQGAHRYPRYHSNLFRGQDAWDPHTHVLLPSQSPQPSPSEVLPTSSNAENATASGVVSPPAPLEPESEPGISIVILLVYRALGGLLPAQFQAERRGARLPQNPVMNSPVVSVAVFRGRNFLRGALVSPINLEFRLLQTANRSKAICVQWDPPGPADQHGMWTARDCELVHRNGSHARCRCSRTGTFGVLMDASPRERLEGDLELLAVFTHVVVAASVTALVLTAAVLLSLRSLKSNVRGIHANVAAALGVAELLFLLGIHRTHNQLLCTVVAILLHYFFLSTFAWLLVQGLHLYRMQVEPRNVDRGAMRFYHALGWGVPAVLLGLAVGLDPEGYGNPDFCWISIHEPLIWSFAGPIVLVIVMNGIMFLLAARTSCSTGQREAKKTSVLRTLRSSFLLLLLVSASWLFGLLAVNHSVLAFHYLHAGLCGLQGLAVLLLFCVLNADARAAWTPACLGKKAAPEETRPAPGPGSGAYNNTALFEESGLIRITLGASTVSSVSSARSGRAQDQDSQRGRSYLRDNVLVRHGSTAEHAEHSLQAHAGPTDLDVAMFHRDAGADSDSDSDLSLEEERSLSIPSSESEDNGRTRGRFQRPLRRAAQSERLLAHPKDVDGNDLLSYWPALGECEAAPCALQAWGSERRLGLDSNKDAANNNQPELALTSGDETSLGRAQRQRKGILKNRLQYPLVPQTRGTPELSWCRAATLGHRAVPAASYGRIYAGGGTGSLSQPASRYSSREQLDLLLRRQLSRERLEEVPVPAPVLHPLSRPGSQERLDTAPARLEPRDRGSTLPRRQPPRDYPGTMAGRFGSRDALDLGAPREWLSTLPPPRRNRDLDPQHPPLPLSPQRPLSRDPLLPSRPLDSLSRISNSRERLDQVPSRHPSREALGPAPQLLRAREDPASGPSHGPSTEQLDILSSILASFNSSALSSVQSSSTPSGPHTTATPSATASALGPSTPRSATSHSISELSPDSEVPRSEGHS

SEQ ID NO: 30 - protein, 3034 aa, Mus musculus; identical to SEQ ID NO: 25

SEQ ID NO: 31 - DNA, 1072 bp, Homo sapiens
ccctgcccctaggttcctggccaacacgtccttccagggccgcacgggccccgtgtgggtgacaggcagctcccaggtacacatgtctcggcactttaaggtgtggagccttcgccgggacccacggggcgccccggcctgggccacggtgggcagctggcgggacggccagctggacttggaaccgggaggtgcctctgcacggcccccgcccccacagggtgcccaggtctggcccaagctgcgtgtggtaacgctgttggaacacccatttgtgtttgcccgtgatccagacgaagacgggcagtgcccagcggggcagctgtgcctggaccctggcaccaacgactcggccaccctggacgcactgttcgccgcgctggccaacggctcagcgccccgtgccctgcgcaagtgctgctacggctactgcattgacctgctggagcggctggcggaggacacgcccttcgacttcgagctgtacctcgtgggtgacggcaagtacggcgccctgcgggacggccgctggaccggcctggtcggggacctgctggccggccgggcccacatggcggtcaccagcttcagtatcaactccgcccgctcacaggtggtggacttcaccagccccttcttctccaccagcctgggcatcatggtgcgggcacgggacacggcctcacccatcggtgcctttatgtggcccctgcactggtccacgtggctgggcgtctttgcggccctgcacctcaccgcgctcttcctcaccgtgtacgagtggcgtagcccctacggcctcacgccacgtggccgcaaccgcagcaccgtcttctcctactcctcagccctcaacctgtgctacgccatcctcttcagacgcaccgtgtccagcaagacgcccaagtgccccacgggccgcctgctcatgaacctctgggccatcttctgcctgctggtgctgtccagctacacggccaacctggctgccgtcatggtcggggacaagaccttcgaggagctgtcggggatccacgaccccaaggtgggcggcctcggggggctgcgggtg

SEQ ID NO: 32 - DNA, 1083 bp, Artificial Sequence
  Consensus Sequence
cnctgccnccnangnncctgnccancncgnnnctnccannnnngcnncnggcncngngngnggtgncangancnccncnggnanncnngncncnncncntnannntgtgnancnnntngccnggncncnngggnngnncngnccnggnncancngnngnnagcnnncnnnncngccnngntnnactnnnggnaccngnagnngncnnngcncgngnccccgnnncnncnnnntgncnngngtnnctggccnaancnncgtnntnnnanngcngnnngnncncccntntgngtnnnngncngnnnccagacgaagacgggcagtgcccagcggggcagctgtgcctggaccctggcaccaacgactcggccaccctggacgcactgttcgccgcgctggccaacggctcagcgccccgtgccctgcgcaagtgctgctacggctactgcattgacctgctggagcggctggcggaggacacgcccttcgacttcgagctgtacctcgtgggtgacggcaagtacggcgccctgcgggacggccgctggaccggcctggtcggggacctgctggccggccgggcccacatggcggtcaccagcttcagtatcaactccgcccgctcacaggtggtggacttcaccagccccttcttctccaccagcctgggcatcatggtgcgggcacgggacacggcctcacccatcggtgcctttatgtggcccctgcactggtccacgtggctgggcgtctttgcggccctgcacctcaccgcgctcttcctcaccgtgtacgagtggcgtagcccctacggcctcacgccacgtggccgcaaccgcagcaccgtcttctcctactcctcagccctcaacctgtgctacgccatcctcttcagacgcaccgtgtccagcaagacgcccaagtgccccacgggccgcctgctcatgaacctctgggccatcttctgcctgctggtgctgtccagctacacggccaacctggctgccgtcatggtcggggacaagaccttcgaggagctgtcggggatccacgaccccaaggnnnncngcntcggntgngggng

SEQ ID NO: 33 - protein, 901 aa, Homo sapiens [sequence omitted]

SEQ ID NO: 34 - protein, 474 aa, Artificial Sequence
  Consensus Sequence
XVXXXXPDEDGQCPAGQLCLDPGTNDSATLDALFAALANGSAPRALRKCCYGYCIDLLERLAEDTPFDFELYLVGDGKYGALRDGRWTGLVGDLLAGRAHMAVTSFSINSARSQVVDFTSPFFSTSLGIMVRARDTASPIGAFMWPLHWSTWLGVFAALHLTALFLTVYEWRSPYGLTPRGRNRSTVFSYSSALNLCYAILFRRTVSSKTPKCPTGRLLMNLWAIFCLLVLSSYTANLAAVMVGDKTFEELSGIHDPKXXXXXXGFRFGTVWESSAEAYIKKSFPDMHAHMRRHSAPTTPRGVAMLTSDPPKLNAFIMDKSLLDYEVSIDADCKLLTVGKPFAIEGYGIGLPQNSPLTSNLSEFISRYKSSGFIDLLHDKWYKMVPCGKRVFAVTETLQMSIYHFAGLFVLLCLGLGSALLSSLGEHAFFRLALPRIRKGSRLQYWLHTSQKIHRALNTEPPEGSKEETAEAEP

SEQ ID NO: 35 - protein, 1135 aa, Rattus norvegicus
MRRLSLWWLLSRVCLLLPPPCALVLAGVPSSSSHPQPCQILKRIGHAVRVGAVHLQPWTTAPRAASRAQEGGRAGAQRDDPESGTWRPPAPSQGARWLGSALHGRGPPGSRKLGEGAGAETLWPRDALLFAVENLNRVEGLLPYNLSLEVVMAIEAGLGDLPLMPFSSPSSPWSSDPFSFLQSVCHTVVVQGVSALLAFPQSQGEMMELDLVSSVLHIPVLSIVRHEFPRESQNPLHLQLSLENSLSSDADVTVSILTMNNWYNFSLLLCQEDWNITDFLLLTENNSKFHLESVINITANLSSTKDLLSFLQVQMDNIRNSTPTMVMFGCDMDSIRQIFEMSTQFGLSPPELHWVLGDSQNVEELRTEGLPLGLIAHGKTTQSVFEYYVQDAMELVARAVATATMIQPELALLPSTMNCMDVKTTNLTSGQYLSRFLANTTFRGLSGSIKVKGSTIISSENNFFIWNLQHDPMGKPMWTRLGSWQGGRIVMDSGIWPEQAQRHKTHFQHPNKLHLRVVTLIEHPFVFTREVDDEGLCPAGQLCLDPMTNDSSMLDRLFSSLHSSNDTVPIKFKKCCYGYCIDLLEQLAEDMNFDFDLYIVGDGKYGAWKNGHWTGLVGDLLSGTANMAVTSFSINTARSQVIDFTSPFFSTSLGILVRTRDTAAPIGAFMWPLHWTMWLGIFVALHITAIFLTLYEWKSPFGMTPKGRNRNKVFSFSSALNVCYALLFGRTAAIKPPKCWTGRFLMNLWAIFCMFCLSTYTANLAAVMVGEKIYEELSGIHDPKLHHPSQGFRFGTVRESSAEDYVRQSFPEMHEYMRRYNVPATPDGVQYLKNDPEKLDAFIMDKALLDYEVSIDADCKLLTVGKPFAIEGYGIGLPPNSPLTSNISELISQYKSHGFMDVLHDKWYKVVPCGKRSFAVTETLQMGIKHFSGLFVLLCIGFGLSILTTIGEHIVHRLLLPRIKNKSKLQYWLHTSQRFHRALNTSFVEEKQPRSKTKRVEKSRWRRWTCKTEGDSELSLFPRSNLGPQQLMVWNTSNLSHDNQRKYIFNDEEGQNQLGTQAHQDIPLPQRRRELPASLTTNGKADSLNVTRSSVIQELSELEKQIQVIRQELQLAVSRKTELEEYQKTNRTCES

SEQ ID NO: 36 - DNA, 1120 bp, Artificial Sequence
  Consensus Sequence
cnctgccnccnangnncctgnccancncgnnnctnccannnnngcnncnggcncngngngnggtgncangancnccncnggnanncnngncncnncncntnannntgtgnancnnntngccnggncncnngggnngnncngnccnggnncancngnngnnagcnnncnnnncngccnngntnnactnnnggnaccngnagnngncnnngcncgngnccccgnnncnncnnnntgncnnggtnnctggccnaancnncgtnntnnnanngcngnnngnncncccntntgngtnnnngncngnnnnccagacgaagacgggcagtgcccagcggggcagctgtgcctggaccctggcaccaacgactcggccaccctggacgcactgttcgccgcgctggccaacggctcagcgccccgtgccctgcgcaagtgctgctacggctactgcattgacctgctggagcggctggcggaggacacgcccttcgacttcgagctgtacctcgtgggtgacggcaagtacggcgccctgcgggacggccgctggaccggcctggtcggggacctgctggccggccgggcccacatggcggtcaccagcttcagtatcaactccgcccgctcacaggtggtggacttcaccagccccttcttctccaccagcctgggcatcatggtgcgggcacgggacacggcctcacccatcggtgcctttatgtggcccctgcactggtccacgtggctgggcgtctttgcggccctgcacctcaccgcgctcttcctcaccgtgtacgagtggcgtagcccctacggcctcacgccacgtggccgcaaccgcagcaccgtcttctcctactcctcagccctcaacctgtgctacgccatcctcttcagacgcaccgtgtccagcaagacgcccaagtgccccacgggccgcctgctcatgaacctctgggccatcttctgcctgctggtgctgtccagctacacggccaacctggctgccgtcatggtcggggacaagaccttcgaggagctgtcggggatccacgaccccaagntgnncnnccncggngnngggctncngntnnggcnnngngngggnnagcngngnccnngg

SEQ ID NO: 37 - protein, 474 aa, Artificial Sequence
  Consensus Sequence
XVXXXXPDEDGQCPAGQLCLDPGTNDSATLDALFAALANGSAPRALRKCCYGYCIDLLERLAEDTPFDFELYLVGDGKYGALRDGRWTGLVGDLLAGRAHMAVTSFSINSARSQVVDFTSPFFSTSLGIMVRARDTASPIGAFMWPLHWSTWLGVFAALHLTALFLTVYEWRSPYGLTPRGRNRSTVFSYSSALNLCYAILFRRTVSSKTPKCPTGRLLMNLWAIFCLLVLSSYTANLAAVMVGDKTFEELSGIHDPKLHHPAQGFRFGTVWESSAEAYIKKSFPDMHAHMRRHSAPTTPRGVAMLTSDPPKLNAFIMDKSLLDYEVSIDADCKLLTVGKPFAIEGYGIGLPQNSPLTSNLSEFISRYKSSGFIDLLHDKWYKMVPCGKRVFAVTETLQMSIYHFAGLFVLLCLGLGSALLSSLGEHAFFRLALPRIRKGSRLQYWLHTSQKIHRALNTEPPEGSKEETAEAEP

SEQ ID NO: 38 - DNA, 1094 bp, Artificial Sequence
  Consensus Sequence
ggttcctggccaacacgtccttccagggccgcacgggccccgtgtgggtgacaggcagctcccaggtacacatgtctcggcactttaaggtgtggagccttcgccgggacccacggggcgccccggcctgggccacggtgggcagctggcgggacggccagctggacttggaaccgggaggtgcctctgcacggcccccgcccccacagggtgcccaggtctggcccaagctgcgtgtggtaacgctgttggaacacccatttgtgtttgcccgtgatccagacgaagacgggcagtgcccagcggggcagctgtgcctggaccctggcaccaacgactcggccaccctggacgcactgttcgccgcgctggccaacggctcagcgccccgtgccctgcgcaagtgctgctacggctactgcattgacctgctggagcggctggcggaggacacgcccttcgacttcgagctgtacctcgtgggtgacggcaagtacggcgccctgcgggacggccgctggaccggcctggtcggggacctgctggccggccgggcccacatggcggtcaccagcttcagtatcaactccgcccgctcacaggtggtggacttcaccagccccttcttctccaccagcctgggcatcatggtgcgggcacgggacacggcctcacccatcggtgcctttatgtggcccctgcactggtccacgtggctgggcgtctttgcggccctgcacctcaccgcgctcttcctcaccgtgtacgagtggcgtagcccctacggcctcacgccacgtggccgcaaccgcagcaccgtcttctcctactcctcagccctcaacctgtgctacgccatcctcttcagacgcaccgtgtccagcaagacgcccaagtgccccacgggccgcctgctcatgaacctctgggccatcttctgcctgctggtgctgtccagctacacggccaacctggctgccgtcatggtcggggacaagaccttcgaggagctgtcggggatccacgaccccaagntgnncnnccncggngnngggctncngntnnggcnnngngngggnnagcngngnccnngg

SEQ ID NO: 39 - protein, 286 aa, Caenorhabditis elegans
RSTLVNKEPDSMLAHMFKDKGVWGNKQDHRGAFLIDRSPEYFEPILNYLRHGQLIVNDGINLLGVLEEARFFGIDSLIEHLEVAIKNSQPPEDHSPISRKEFVRFLLATPTKSELRCQGLNFSGADLSRLDLRYINFKMANLSRCNLAHANLCCANLERADLSGSVLDCANLQGVKMLCSNAEGASLKLCNFEDPSGLKANLEGANLKGVDMEGSQMTGINLRVATLKNAKLKNCNLRGATLAGTDLENCDLSGCDLQEANLRGSNVKGAIFEEMLTPLHMSQSVR

SEQ ID NO: 40 - DNA, 903 bp, Rattus norvegicus
agacttctagcctgcccctctaacgtgatggccgtggacatagaatacagctacagcagtatggccccttctctgcgcagagagcgcttcaccttcaagatctcccccaaactgaacaagccactgaggccttgtattcagctgggcagcaaggatgaagccggcagaatggtggcccccacagtacaggagaagaaggtgaagaagcgggtgtccttcgccgacaaccaggggctggccctaacaatggtgaaagtgttctcggaattcgatgacccactagatattccgtttaacatcactgagctcctagacaacatcgtgagtctgacgacagcagagagtgagagctttgttttggattttccgcagccttctgcagattacttagactttagaaatcggcttcagaccaaccatgtctgcctcgaaaactgcgtgctgaaggagaaagccatcgcgggcaccgtcaaggtccagaacctggcattcgagaaggttgtgaagatcagcatgacattcgatacctggaaaagcttcacagacttcccttgtcagtatgtgaaggacacttacgctggttcagacagggacacattctcctttgatatcagcctaccggagaaaatccagtcttatgaaagaatggagttcgccgtgtgctacgagtgtaacggccagtcgtactgggacagcaacaaaggcaaaaactacaggatcaccagggccgaactcagatccacccagggaatgactgagccgtacaatgggccggattttggaatctcttttgaccagttcgggagccctcggtgttccttcggcctgtttccagagtggcctagttatctggggtatgaaaagctggggccctattactagtgagttgactgcagttgacag

SEQ ID NO: 41 - DNA, 906 bp, Artificial Sequence
  Consensus Sequence
agnnttctagcctgnncntctancnnnntgatggcngtggacatngantacagntacancngnatggcnccttcnntgcgcnnagagngnttnnccttnaagatctcnccnaancnnancaanccactgaggccttgtattcagctgngcagcaagnatgaagccngnngaatggtggccccnncngtncaggagaagaaggtgaanaagcgggtgtccttcgcngacaaccaggggctggccctnacaatggtnaaagtgttctcggaattcgatgacccnctagatatnccnttnaacatcacngagctcctagacaacatngtgagnntgacgacagcagagagngagagctttgttntggattttncncagccntctgcagattacttagactttagaaatcgncttcagnccnaccangtctgcctnganaactgngtgctnaagganaangccatngcnggcacngtnaaggtncagaacctngcattngagaagnnngtgaanatnagnatgacnttcganacctggaanagctncacagacttnccttgtcagtangtgaaggacacttangcnggttcagacagggacacnttctccttnganatcagcntnccngagaanatncagtcttatgaaagaatggagttngcngtgtnctacgagtgnaanggncagncgtactgggacagcaacanaggcaanaactanaggatcancngggcnganntnanatcnacccagggaatgacnnagccnnacantggnccggatttnggaatntcntttgaccagttcggnagccctcggtgttcctnnggnctgtttccagagtggccnagttanntnggntatgaaaagctngggccctantactagtgannnnnctgcagntgacag

SEQ ID NO: 42 - protein, 284 aa, Rattus norvegicus
MAVDIEYSYSSMAPSLRRERFTFKISPKLNKPLRPCIQLGSKDEAGRMVAPTVQEKKVKKRVSFADNQGLALTMVKVFSEFDDPLDIPFNITELLDNIVSLTTAESESFVLDFPQPSADYLDFRNRLQTNHVCLENCVLKEKAIAGTVKVQNLAFEKVVKIRMTFDTWKSFTDFPCQYVKDTYAGSDRDTFSFDISLPEKIQSYERMEFAVCYECNGQSYWDSNKGKNYRITRAELRSTQGMTEPYNGPDFGISFDQFGSPRCSFGLFPEWPSYLGYEKLGPYY

SEQ ID NO: 43 - protein, 282 aa, Artificial Sequence
  Consensus Sequence
MAVDIEYXYXXMAPSLRXERFXFKISPKXKPLRPCIQLXSKXEAXXMVAPXVQEKKVKKRVSFADNQGLALTMVKVFSEFDDPLDXPFNITELLDNIVSLTTAESESFVLDFXQPSADYLDFRNRLQXHVCLENCVLKXKAIAGTVKVQNLAFEKXVKIRMTFDTWKSXTDFPCQYVKDTYAGSDRDTFSFDISLPEKIQSYERMEFAVXYECNGQXYWDSNXGKNYRIXRAELXSTQGMTXPXXGPDXGISFDQFGSPRCSXGLFPEWPSYLGYEKLGPYY

SEQ ID NO: 44 - protein, 294 aa, Mus musculus
MAMRICLAHSPPLKSFLGPYNGFQRRNFVNKLKPLKPCLSVKQEAKSQSEWKSPHNQAKKRVVFADSKGLSLTAIHVFSDLPEEPAWDLQFDLLDLNDISSSLKLHEEKNLVFDFPQPSTDYLSFRDRFQKNFVCLENCSLEDRTVTGTVKVKNVSFEKKVQVRITFDTWKTYTDVDCVYMKNVYSSSDSDTFSFAIDLPRVIPTEEKIEFCISYHANGRIFWDNNEGQNYRIVHVQWKPDGVQTQVAPKDCAFQQGPPKTEIEPTVFGSPRLASGLFPEWQSWGRVENLTSYR

SEQ ID NO: 45 - protein, 312 aa, Homo sapiens
MIQVLDPRPLTSSVMPVDVAMRLCLAHSPPVKSFLGPYDEFQRRHFVNKLKPLKSCLNIKHKAKSQNDWKCSHNQAKKRVVFADSKGLSLTAIHVFSDLPEEPAWDLQFDLLDLNDISSALKHHEEKNLILDFPQPSTDYLSFRSHFQKNFVCLENCSLQERTVTGTVKVKNVSFEKKVQIRITFDSWKNYTDVDCVYMKNVYGGTDSDTFSFAIDLPPVIPTEQKIEFCISYHANGQVFWDNNDGQNYRIVHVQWKPDGVQTQMAPQDCAFHQTSPKTELESTIFGSPRLASGLFPEWQSWGRMENLASYR

SEQ ID NO: 46 - protein, 70 aa, Homo sapiens
MPPNLTGYYRFVSQKNMEDYLQALNISLAVRKIALLLKPDKEIEHQGNHMTVRTLSTFRNYTVQFDVGVQ

SEQ ID NO: 47 - protein, 70 aa, Artificial Sequence
  Consensus Sequence
MPPNLTGYYRFVSQKNMEDYLQALNISLAVRKIALLLKPDKEIEHQGNHMTVRTLSTFRNYTXQFDVGVX

SEQ ID NO: 48 - protein, 135 aa, Homo sapiens
MPPNLTGYYRFVSQKNMEDYLQALNISLAVRKIALLLKPDKEIEHQGNHMTVRTLSTFRNYTLQFDVGVEFEEDLRSVDGRKCQTIVTWEEEHLVCVQKGEVPNRGWRHWLEGEMLYLELTARDAVCEQVFRKVR

SEQ ID NO: 49 - protein, 135 aa, Artificial Sequence
  Consensus Sequence
MPPNLTGYYRFVSQKNMEDYLQALNISLAVRKIALLLKPDKEIEHQGNHMTVRTLSTFRNYTXQFDVGVEFEEDLRSVDGRKCQTIVTWEEEHLVCVQKGEVPNRGWRHWLEGEXLYLELTARDAVCEQVFRKVR

(19) United States
(12) Patent Application Publication
(10) Pub. No.: US 2004/0086931 A1
(43) Pub. Date: May 6, 2004

(54) Polypeptides and nucleic acids encoding same

(76) Inventors: Steven K. Spaderna, Berlin, CT (US); Kerry E. Quinn, Hamden, CT (US); Richard A. Shimkets, West Haven, CT (US); Muralidhara Padigaru, Branford, CT (US); Kimberly A. Spytek, New Haven, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/701,283

(22) Filed: Nov. 3, 2003

Related U.S. Application Data
(62) Division of application No. 09/737,149, filed on Dec. 14, 2000.
Application No. 09/737,149 is a continuation-in-part of application No. 09/735,981, filed on Dec. 13, 2000.
(60) Provisional application No. 60/170,564, filed on Dec. 14, 1999.
(60) Provisional application No. 60/173,165, filed on Dec. 27, 1999.
(60) Provisional application No. 60/173,362, filed on Dec. 27, 1999.
(60) Provisional application No. 60/173,544, filed on Dec. 29, 1999.
(60) Provisional application No. 60/174,404, filed on Jan. 5, 2000.
(60) Provisional application No. 60/223,929, filed on Aug. 9, 2000.

(51) Int. Cl. C12Q 1/68; C07H 21/04; C07K 14/705; C12P 21/02; A61K 48/00; A61K 38/17

(52) U.S. Cl. 435/6; 435/69.1; 435/320.1; 435/325; 514/12; 514/44; 530/350; 536/23.5

(57) ABSTRACT

The present invention provides novel isolated MEMX polynucleotides and polypeptides encoded by the MEMX polynucleotides. Also provided are the antibodies that immunospecifically bind to a MEMX polypeptide or any derivative, variant, mutant or fragment of the MEMX polypeptide, polynucleotide or antibody. The invention additionally provides methods in which the MEMX polypeptide, polynucleotide and antibody are utilized in the detection and treatment of a broad range of pathological states, as well as to other uses.

### RELATED APPLICATIONS

This application is a divisional application of U.S. Ser. No. 09/737,149; which is a continuation-in-part of U.S. Ser. No. 09/735,981, filed Dec. 13, 2000, which claims priority to U.S. S No. 60/170,564, filed Dec. 14, 1999; U.S. S No. 60/173,165, filed Dec. 27, 1999; U.S. S No. 60/173,362, filed Dec. 27, 1999; U.S. S No. 60/173,544, filed Dec. 29, 1999; U.S. S No. 60/174,404, filed Jan. 5, 2000; and U.S. S No. 60/223,929, filed Aug. 9, 2000 the contents of which are incorporated herein by reference in their entireties.

### FIELD OF THE INVENTION

The invention generally relates to nucleic acids and polypeptides encoded therefrom. More specifically, the invention relates to nucleic acids encoding membrane bound and secreted polypeptides, as well as vectors, host cells, antibodies, and recombinant methods for producing these nucleic acids and polypeptides.

### BACKGROUND OF THE INVENTION

Seven-Pass Transmembrane Receptor

Seven-pass transmembrane proteins are transmembranal proteins with seven α-helices, comprising mostly hydrophobic residues, which serve to facilitate membrane anchoring (see, e.g., Muller, 2000. Curr. Med. Chem. 7: 861-888) and are believed to accommodate the binding site for low-molecular weight ligands.

The most well-characterized of the seven-pass transmembrane receptor proteins are the guanine nucleotide-binding signal-transducing protein (G-protein)-coupled receptors (GPCR) which transduce chemical signals through the cytoplasmic membrane by the activation of intracellular G-proteins. See, e.g., Watson and Arkinstall, THE G-PROTEIN LINKED RECEPTORS, Academic Press, San Diego, Calif., 1994, pp. 1-294. In addition, GPCRs constitute the most prominent family of validated drug targets within biomedical research, as approximately 60% of all approved drugs elicit their therapeutic effects by selectively interacting members of this family of proteins and serve as key molecular targets for therapeutic intervention in a host of disease states.

GPCRs transduce extracellular signals that modulate the activity of a wide variety of biological processes, such as neurotransmission, chemoattraction, cardiac function, olfaction, and vision. Hundreds of GPCRs signal through one or more of these G proteins in response to a large variety of stimuli including photons, neurotransmitters, and hormones of variable molecular structure. GPCRs function as a diverse family of regulatory GTPases which mediate their intracellular actions through the activation of guanine nucleotide-binding signal-transducing proteins (G proteins), that, in the GTP-bound state, bind and activate downstream membrane-localized effectors. The mechanisms by which these ligands provoke activation of the receptor/G-protein system are highly complex and multifactorial. Prominent members of GPCRs include, e.g., angiotensin II, CCK/gastrin, interleukin 8, endothelin, and the like. See, e.g., van Neuren, 1999. J. Recept. Signal Transduct. Res. 9:341-353.

The GPCR superfamily is evolutionarily conserved and structurally characterized by its possessing putative seven-transmembrane (TM) domains with an extracellular amino-terminus and a cytoplasmic carboxyl-terminus. GPCRs are composed of several independent folding units, with the transmembrane domains arranged in a barrel-like structure with a tightly packed core. The universal adoption of the conserved seven-TM structure by GPCRs, which consequently confers three intracellular and three extracellular loops along with a TM core, generally is speculated as the minimum necessity to achieve their structural stability and functional diversity. None of nearly 2,000 GPCRs identified in prokaryotes and eukaryotes to date are known to contain fewer than seven TM domains.

In a recent study, alignment of the primary sequences demonstrated a high degree of homology within the GPCR transmembrane regions. Three-dimensional (3D) models of 39 GPCRs were generated using the refined model of bacteriorhodopsin as a template. Five cationic neurotransmitter receptors (i.e., serotonergic 5-HT2, dopaminergic D2, muscarinic m2, adrenergic alpha 2, and beta 2 receptors) were taken as prototypes and studied in detail. The 3D models of the cationic neurotransmitter receptors, together with their primary structure comparison, indicate that the agonist binding site is located near the extracellular face of the receptor and involves residues of the membrane-spanning helices 3, 4, 5, 6, and 7. The binding site consists of a negatively-charged Asp located at the middle of transmembrane helix 3 and a hydrophobic pocket containing conserved aromatic residues on helices 4, 5, 6, and 7. In addition, all the GPCRs were shown to possess invariant hinge residues, which are thought to be responsible for a conformational change during agonist binding and therefore influence dissociation and association of G-proteins to the receptors. Modulation of the coupling of the G-protein is due to conformation changes within this region via hydrophobic interactions and hydrogen bonding. The information of an extracellularly occurring receptor-ligand recognition event is transferred through conformational rearrangements within the transmembranal portion of GPCR to the intracellular compartment. Thus, GPCRs establish a functional and unidirectional link between the exterior of a cell and its cytoplasm.

Generally, GPCR activation is followed rapidly by a loss of responsiveness, termed desensitization, which is then followed by a period of recovery or resensitization. These changes in signaling potential are tightly regulated, primarily via mechanisms that involve GPCR phosphorylation and trafficking to distinct locations within the cell.

Glutamate and Aspartate Receptors

Glutamate and Aspartate receptors abound in the Central Nervous System (CNS), eliciting responses both by ionotropic and metabotropic responses. Included within the metabotrophic response class are glutamate receptors; which are generally comprised of seven, single-chain transmembrane-spanning proteins. Many cDNAs encoding metabotropic receptors, as well as ionotropic receptors for N-methyl-D-aspartate, have been identified in recent years. The diversity of receptor types has also been found to markedly increase as a result of alternative splicing processes and even by single-base editing of mRNAs. See, e.g., Gilman and Goodman's The Pharmacological Basis of Therapeutics, Ninth Ed., Hardman, J G, et al. (eds.) McGraw-Hill, New York, 1996, pages 278-282.

Recently there has been interest in investigating the role of glutamate receptors in the pathophysiology of schizophrenia. Indeed, the hyperdopaminergic theory of schizophrenia can explain only the positive symptoms of schizophrenia, whereas the glutamate hypothesis may provide a more comprehensive view of the illness. Noorbala, et al. (Piracetam in the treatment of schizophrenia: implications for the glutamate hypothesis of schizophrenia. PMID: 10583700) undertook a trial to investigate whether the combination of haloperidol with piracetam, a nootropic agent that modulates the glutamate receptor positively, was more effective than haloperidol alone in treating the disease. They examined thirty patients who met the DSM IV criteria for schizophrenia. Patients were allocated in a random fashion, 14 received both haloperidol (30 mg/day) and piracetam (3200 mg/day), and 16 patients received only haloperidol (30 mg/day) plus placebo. It was found that both protocols significantly decreased the score of the positive symptoms, the negative symptoms, the general psychopathological symptoms and the total score of PANSS scale over the trial period. Nevertheless, these workers also demonstrated that the combination of haloperidol and piracetam showed a significant superiority over haloperidol alone in the treatment of schizophrenic patients. They concluded that piracetam, a member of the nootropic class of drugs and a positive modulator of the glutamate receptor, may be of therapeutic benefit in treating schizophrenic patients in combination with typical neuroleptic agents.

Excessive activity of excitatory amino acids released after head trauma has also been demonstrated to contribute to progressive injury in animal models and human studies. See, e.g., Morris, et al., 1999. J Neurosurg 91(5): 737-743. Several pharmacological agents that act as antagonists to the glutamate receptor have shown promise in limiting this progression. The efficacy of the N-methyl-D-aspartate receptor antagonist Selfotel (CGS 19755) was evaluated in two parallel studies of severely head injured patients, defined as patients with post resuscitation Glasgow Coma Scale scores of 4 to 8. The Selfotel trial was terminated prior to completion, however, because of severe adverse effects on some of the subjects. The results of this trial demonstrate the need for a better understanding of the properties of the glutamate receptors in the brain, and of the need for discovering more effective agonists and antagonists of this receptor.

Potassium Channel

The potassium channel mediates the voltage-dependent potassium ion permeability of excitable membranes. Depending upon whether the protein assumes an opened or closed conformation in response to the voltage difference across the membrane, the protein forms a potassium-selective channel through which potassium ions may pass in accordance with their electrochemical gradient.

The potassium channel has been shown to be an integral membrane protein. The segment s4 is probably the voltage-sensor and is characterized by a series of positively charged amino acids at every third position. Additionally, the tail may be important in modulation of channel activity and/or targeting of the channel to specific sub-cellular compartments. This channel protein belongs to the delayed rectifier class, and to the Shaw potassium channel subfamily.

IKr (potassium ion channel, rapid response) blockade is ineffective in preventing ventricular fibrillation elicited by the interaction between acute myocardial ischemia and elevated sympathetic activity. This depends, in-part, upon the fact that adrenergic activation offsets more than 50% of the action potential prolonging effect of IKr blockade, and thus impairs its primary mechanism of action. The antifibrillatory effect of ersentilide (CK-3579), a novel antiarrhythmic agent which combines blockade of the rapid component of the delayed rectifier potassium channel (IKr) with relatively weak beta-adrenergic blockade, has been examined in a conscious canine model of lethal arrhythmias. See, Adamson, et al., 1998. Cardiovascular Res. 40(1): 56-63). Ersentilide was tested in 19 dogs with a healed myocardial infarction (MI) undergoing two minutes of circumflex artery occlusion (CAO) during sub-maximal treadmill exercise. Epicardial monophasic action potential duration was measured before and after ersentilide in 8 anesthetized open chest dogs at baseline and during stimulation of the left stellate ganglion at constant paced heart rate. In the control tests 13 of the 19 dogs had ventricular fibrillation (VF) during the exercise and ischemia test, 6 did not. During a subsequent exercise test, ersentilide prevented VF in 820% (11 of 13) of the high-risk animals and showed no pro-arrhythmic effects in the 6 dogs without arrhythmias in the initial test. Ersentilide lowered heart rate at all levels of exercise and during acute myocardial ischemia. The anti-fibrillatory effect was maintained in 3 of 4 dogs in which heart rate was kept at control levels by atrial pacing. Ersentilide also was found to prolonged left ventricular monophasic action potential duration by 30% (from 179+/−6 ms to 233+/−5 ms, p<0.001) at a 360 ms cycle length and completely prevented its shortening during sympathetic stimulation. Thus, these authors concluded that the combination of IKr and weak beta-adrenergic blockade, using ersentilide, represents a very effective and safe anti-arrhythmic intervention able to overcome the limitations present in drugs devoid of any anti-adrenergic effect. Such a combination may be very useful in the management of post-myocardial infarction patients at high arrhythmic risk.

Nair and Grant (1997. Cardiovascular Drugs Ther. 11(2): 149-167) reviewed antiarrhythmic drugs. The goal of developing an antiarrhythmic agent effective against malignant ventricular arrhythmias while maintaining a low side-effect profile was evaluated as remaining elusive. In this study, the class III drugs, amiodarone and sotalol, were regarded as the best available agents. However, both drugs possess properties outside the realm of a pure class III effect, and their use is limited by a variety of dose-related side effects. There are several drugs with more selective class III properties currently in development.

The aforementioned review by Nair and Grant (1997) provides an overview of the optimal characteristics of an effective theoretical class III drug and a summary of the properties of a number of class III drugs under active investigation. An ideal class III antiarrhythmic agent for a reentrant arrhythmia should provide use-dependent prolongation of the action potential duration with slow onset and rapid offset kinetics. This drug would prolong the effective refractory period of cardiac tissue selectively at the rapid heart rates achieved during ventricular tachycardia or fibrillation with a delayed onset of action, and a rapid resolution of its effects on resumption of physiologic heart rates. With little effect on the refractory period at normal or slow heart rates, the ability to induce torsade de pointes would be lessened. In contrast to these ideal properties, most currently available and investigational agents have a reverse use-dependent effect on the action potential duration, producing more effects on the refractory period at slower heart rates. This property results in part from preferential block of the rapidly activating component of the delayed rectifier potassium channel (IKr), with little or no effect on the slowly activating component (IKs). The development of a drug with favorable blocking kinetics that selectively blocks IKs may results in lower proarrhythmic events while still maintaining effective antiarrhythmic properties.

Protein Phosphatase I

Protein phosphatase 1 is believed to act as a scaffold for the localization of critical enzymes in glycogen metabolism, including phosphorylase b, glycogen synthase and phosphorylase kinase. The enzyme is expressed predominantly in insulin-sensitive tissues and was found to mediate the hormonal control of glycogen accumulation in intact cells.

Hepatic glycogen synthesis is impaired in insulin-dependent diabetic rats and in adrenalectomized starved rats, and although this is known to be due to defective activation of glycogen synthase by glycogen synthase phosphatase, the underlying molecular mechanism has not been delineated. Glycogen synthase phosphatase comprises the catalytic subunit of protein phosphatase 1 (PP1) complexed with the hepatic glycogen-binding subunit, termed GL. In liver extracts of insulin-dependent diabetic and adrenalectomized starved rats, the level of GL was shown by immunoblotting to be substantially reduced compared with that in control extracts, whereas the level of PP1 catalytic subunit was not affected by these treatments. See, Doherty, et al., 1998. Biochem. J. 333: 253-257. Insulin administration to diabetic rats restored the level of GL and prolonged administration raised it above the control levels, whereas re-feeding partially restored the GL level in adrenalectomized starved rats. The regulation of GL protein levels by insulin and starvation/feeding was shown to correlate with changes in the level of the GL mRNA, indicating that the long-term regulation of the hepatic glycogen-associated form of PP1 by insulin, and hence the activity of hepatic glycogen synthase, is predominantly mediated through changes in the level of the GL mRNA. (PMID: 9657963, UI: 98324884).

Retinol-Binding Protein

Retinol-binding protein (RBP) is the specific carrier for retinol (vitamin A1) in the blood. Low RBP level in the blood has been found to be associated with low serum retinol level in keratomalacia patients. Familial hypo-RB proteinemia has been found to predispose the proband child to keratomalacia during measles infection, despite good nutrition. See, e.g., Attard-Montalto, et al. described a girl with intermittent orange discoloration of her palms, soles, and face and with carotenemia associated with persistently low levels of both vitamin A and serum-specific retinol-binding protein. These authors postulated that the low serum retinol-binding protein concentration resulted in the slow uptake and release of vitamin A by the liver. The conversion of carotene to vitamin A was consequently inhibited and this resulted in hypercarotenemia. Vitamin A supplements were unable to raise the serum vitamin A concentration and did not relieve the carotenemia.

Seeliger, et al., reported the ocular phenotype in retinol deficiency due to a hereditary defect in retinol-binding protein synthesis. Two affected sisters, aged 17 and 13 years, were compound heterozygous for missense mutations in the RBP4 gene. Each affected sister had had night vision problems since early childhood but was otherwise well. Visual acuities were slightly reduced: 20/40 in the 17 year old and 20/25 in the 13 year old. Both affected sibs had no detectable serum RBP, retinol levels less than 20% of normal, and normal retinyl esters.

RBP gene has been mapped to the long arm of chromosome 10 and is homologous to bovine beta-lactoglobulin.

### SUMMARY OF THE INVENTION

The invention is based, in part, upon the discovery of a novel polynucleotide sequences encoding novel polypeptides. Nucleic acids encoding these polypeptides, and derivatives and fragments thereof, will hereinafter be collectively designated as “MEMX”.

Accordingly, in one aspect, the invention provides an isolated nucleic acid molecule that includes the sequence of SEQ ID NO:1, 3, 5 7, 9, 11, 13, or 15, or a fragment, homolog, analog or derivative thereof. The nucleic acid can include, e.g., a nucleic acid sequence encoding a polypeptide at least 80% identical to a polypeptide that includes the amino acid sequences of SEQ ID NO: 2, 4, 6, 8, 10, 12, 14, or 16. The nucleic acid can be, e.g., a genomic DNA fragment, or a cDNA molecule.

Also included in the invention is a vector containing one or more of the nucleic acids described herein, and a cell containing the vectors or nucleic acids described herein.

The invention is also directed to host cells transformed with a vector comprising any of the nucleic acid molecules described above.

In another aspect, the invention includes a pharmaceutical composition that includes an MEMX nucleic acid and a pharmaceutically acceptable carrier or diluent.

In a further aspect, the invention includes a substantially purified MEMX polypeptide, e.g., any of the MEMX polypeptides encoded by an MEMX nucleic acid, and fragments, homologs, analogs, and derivatives thereof. The invention also includes a pharmaceutical composition that includes an MEMX polypeptide and a pharmaceutically acceptable carrier or diluent.

In still a further aspect, the invention provides an antibody that binds specifically to an MEMX polypeptide. The antibody can be, e.g., a monoclonal or polyclonal antibody, and fragments, homologs, analogs, and derivatives thereof. The invention also includes a pharmaceutical composition including MEMX antibody and a pharmaceutically acceptable carrier or diluent. The invention is also directed to isolated antibodies that bind to an epitope on a polypeptide encoded by any of the nucleic acid molecules described above.

The invention also includes kits comprising any of the pharmaceutical compositions described above.

The invention further provides a method for producing an MEMX polypeptide by providing a cell containing an MEMX nucleic acid, e.g., a vector that includes an MEMX nucleic acid, and culturing the cell under conditions sufficient to express the MEMX polypeptide encoded by the nucleic acid. The expressed MEMX polypeptide is then recovered from the cell. Preferably, the cell produces little or no endogenous MEMX polypeptide. The cell can be, e.g., a prokaryotic cell or eukaryotic cell.

The invention is also directed to methods of identifying an MEMX polypeptide or nucleic acid in a sample by contacting the sample with a compound that specifically binds to the polypeptide or nucleic acid, and detecting complex formation, if present.

The invention further provides methods of identifying a compound that modulates the activity of an MEMX polypeptide by contacting an MEMX polypeptide with a compound and determining whether the MEMX polypeptide activity is modified.

The invention is also directed to compounds that modulate MEMX polypeptide activity identified by contacting an MEMX polypeptide with the compound and determining whether the compound modifies activity of the MEMX polypeptide, binds to the MEMX polypeptide, or binds to a nucleic acid molecule encoding an MEMX polypeptide.

In another aspect, the invention provides a method of determining the presence of or predisposition of an MEMX-associated disorder in a subject. The method includes providing a sample from the subject and measuring the amount of MEMX polypeptide in the subject sample. The amount of MEMX polypeptide in the subject sample is then compared to the amount of MEMX polypeptide in a control sample. An alteration in the amount of MEMX polypeptide in the subject protein sample relative to the amount of MEMX polypeptide in the control protein sample indicates the subject has a tissue proliferation-associated condition. A control sample is preferably taken from a matched individual, i.e., an individual of similar age, sex, or other general condition but who is not suspected of having a tissue proliferation-associated condition. Alternatively, the control sample may be taken from the subject at a time when the subject is not suspected of having a tissue proliferation-associated disorder. In some embodiments, the MEMX is detected using an MEMX antibody.

In a further aspect, the invention provides a method of determining the presence of or predisposition of an MEMX-associated disorder in a subject. The method includes providing a nucleic acid sample, e.g., RNA or DNA, or both, from the subject and measuring the amount of the MEMX nucleic acid in the subject nucleic acid sample. The amount of MEMX nucleic acid sample in the subject nucleic acid is then compared to the amount of an MEMX nucleic acid in a control sample. An alteration in the amount of MEMX nucleic acid in the sample relative to the amount of MEMX in the control sample indicates the subject has a tissue proliferation-associated disorder.

In a still further aspect, the invention provides a method of treating or preventing or delaying an MEMX-associated disorder. The method includes administering to a subject in which such treatment or prevention or delay is desired an MEMX nucleic acid, an MEMX polypeptide, or an MEMX antibody in an amount sufficient to treat, prevent, or delay a tissue proliferation-associated disorder in the subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1: illustrates the nucleotide sequence of the Seven-Pass Transmembrane Receptor-Like Protein of the invention [SEQ ID NO:1]. The start and stop codons are shown in bold font.

FIG. 2: illustrates the amino acid sequence [SEQ ID NO:2] encoded by the coding sequence shown in FIG. 1.

FIG. 3: illustrates the BLASTN identity searches leading to the nucleic acid sequence [SEQ ID NO:1].

FIG. 4: illustrates the BLASTX identity search for the amino acid sequence [SEQ ID NO:2].

FIG. 5: illustrates the BLASTP identity search for the amino acid sequence [SEQ ID NO:2].

FIG. 6: illustrates the ClustalW alignment of the amino acid sequence [SEQ ID NO:2].

FIG. 7: illustrates the nucleotide sequence, including the sequence encoding a glutamate receptor variant (21659259 EXT 1) of the invention [SEQ ID NO:3]. The start and stop codons are shown in bold font.

FIG. 8: illustrates the amino acid sequence [SEQ ID NO:4] encoded by the coding sequence of 21659259 EXT 1 shown in FIG. 7.

FIG. 9: illustrates the BLASTN identity searches leading to the nucleic acid sequence [SEQ ID NO:3] of variant 21659259 EXT 1.

FIG. 10: illustrates the BLASTX identity search for the amino acid sequence [SEQ ID NO:4] of variant 21659259 EXT 1.

FIG. 11: illustrates the ClustalW alignment of variant 21659259 EXT 1.

FIG. 12: illustrates the nucleotide sequence, including the sequence encoding a glutamate receptor variant (21659259 EXT 2) of the invention [SEQ ID NO:5].

FIG. 13: illustrates the amino acid sequence [SEQ ID NO:6] encoded by the coding sequence of 21659259EXT 2 of FIG. 12.

FIG. 14: illustrates the BLASTN identity searches lea ding to the nucleic acid sequence [SEQ ID NO:5] of variant 21659259 EXT 2.

FIG. 15: illustrates the BLASTX identity search for the amino acid sequence [SEQ ID NO:6] of variant 21659259 EXT 2.

FIG. 16: illustrates the ClustalW alignment of variant 21659259 EXT 2.

FIG. 17: illustrates the nucleotide sequence, including the sequence encoding a glutamate receptor variant (21659259 EXT 3) of the invention [SEQ ID NO:7].

FIG. 18: illustrates the amino acid sequence [SEQ ID NO:8] encoded by the coding sequence of 21659259 EXT 3 of FIG. 17.

FIG. 19: illustrates the BLASTN identity searches leading to the nucleic acid sequence [SEQ ID NO:7] of variant 21659259 EXT 3.

FIG. 20: illustrates the BLASTX identity search for the amino acid sequence [SEQ ID NO:8] of variant 21659259 EXT 3.

FIG. 21: illustrates the ClustalW alignment of variant 21659259 EXT 3.

FIG. 22: illustrates the ClustalW alignment of the three splice variants of the glutamate receptor of the present invention.

FIG. 23: illustrates the nucleotide sequence [SEQ ID NO:9] of the potassium channel protein of the invention. A putative untranslated region 5′ to the start codon is shown by underlining, whereas the start and termination codons are shown in bold font.

FIG. 24: illustrates the amino acid sequence [SEQ ID NO:10] encoded by the coding sequence shown in FIG. 23.

FIG. 25: illustrates the BLASTX identity search for the protein of the invention [SEQ ID NO:10].

FIG. 26: illustrates the nucleotide sequence including the sequence encoding the phosphatase 1-like protein of the invention [SEQ ID NO:11]. Putative untranslated regions 5′ to the start codon and 3′ to termination codon are shown by underlining, and the start and stop codons are shown in bold font.

FIG. 27: illustrates the amino acid sequence [SEQ ID NO:12] encoded by the coding sequence shown in FIG. 26.

FIG. 28: illustrates BLASTN identity search for the nucleic acid encoding the phosphatase 1-like protein of the invention.

FIG. 29: illustrates the BLASTX identity search for the phosphatase 1-like protein of the invention.

FIG. 30: illustrates the ClustalW alignment of the phosphatase 1-like protein of the invention.

FIG. 31: illustrates the nucleotide sequence [SEQ ID NO:13], including the sequence encoding the protein resembling retinol-binding protein, of the invention. Putative untranslated regions 5′ to the start codon and 3′ to the termination codon are shown by underlining, and the start and stop codons are shown in bold font.

FIG. 32: illustrates the amino acid sequence [SEQ ID NO:14] encoded by the coding sequence shown in FIG. 31.

FIG. 33: illustrates the BLASTX identity search for the retinol-binding-like protein of the invention shown in FIG. 32.

FIG. 34: illustrates the nucleotide sequence [SEQ ID NO:15], including the sequence encoding the protein resembling retinol-binding protein, of the invention.

FIG. 35: illustrates the amino acid sequence [SEQ ID NO:16] encoded by the coding sequence shown in FIG. 34.

FIG. 36: illustrates the BLASTP identity search for the retinol-binding-like protein of the invention shown in FIG. 35.

FIG. 37: illustrates the ClustalW alignment of the retinol-binding-like protein of the invention shown in FIG. 35.

### DETAILED DESCRIPTION OF THE INVENTION

The present invention provides novel nucleotides and polypeptides encoded thereby. Included in the invention are the novel nucleic acid sequences and their polypeptides. The sequences are collectively designated as “MEMX nucleic acids” or “MEMX polynucleotides” and the corresponding encoded polypeptides are referred to as “MEMX polypeptides” or “MEMX proteins.” Unless indicated otherwise, “MEMX” is meant to refer to any of the novel sequences disclosed herein. Table 1, below, provides a summary of the MEMX nucleic acids and their encoded polypeptides.

TABLE 1

| | | SEQ | SEQ | |
|---|---|---|---|---|
| MEMX | | ID NO: | ID NO: | |
| Assign- | Internal | (nucleic | (poly- | |
| ment | Identification | acid) | peptide) | Homology |
| 1 | Construct of | 1 | 2 | Seven-Pass |
| | AL021392, | | | Transmembrane |
| | AL031588, and | | | Receptor Protein |
| | AL031597 | | | |
| 2 | 21659259 EXT 1 | 3 | 4 | Glutamate Receptor |
| 3 | 21659259 EXT 2 | 5 | 6 | Glutamate Receptor |
| 4 | 21659259 EXT 3 | 7 | 8 | Glutamate Receptor |
| 5 | 16418841 | 9 | 10 | Potassium Channel |
| | | | | Protein |
| 6 | AC016485_A | 11 | 12 | Phosphatase I Protein |
| 7 | AC018653_A | 13 | 14 | |
| | | | | Protein |
| 8 | AC18653A da1 | 15 | 16 | Retinol-Binding |
| | | | | Protein |

MEMX nucleic acids and their encoded polypeptides are useful in a variety of applications and contexts. The various MEMX nucleic acids and polypeptides according to the invention are useful as members of the protein families according to the presence of domains and sequence relatedness to previously described proteins. Additionally, MEMX nucleic acids and polypeptides can also be used to identify proteins that are members of the family to which the MEMX polypeptides belong.

For example, MEM1 is homologous to members of the Seven-Pass Transmembrane Receptor Protein family of proteins. Thus, the MEM1 nucleic acids and polypeptides, antibodies and related compounds according to the invention will be useful in therapeutic and diagnostic applications in immunotherapy, viral infections, neurological disorders (e.g., Alzheimer's disease or Parkinson's disease), cancer (e.g., breast or neuroblastoma), nephrology, and female reproductive health.

MEM2, MEM3, and MEM4 are homologous to members of the Glutamate Receptor family of proteins. Thus, the MEM2 through MEM4 nucleic acids and polypeptides, antibodies and related compounds according to the invention will be useful in therapeutic and diagnostic applications targeted to lung and/or brain. In brain, it may serve as a target receptor for treating schizophrenia or reducing neuronal damage following head injury.

MEM5 is homologous to members of the Potassium Channel Protein family of proteins. Thus, the MEM5 nucleic acids and polypeptides, antibodies and related compounds according to the invention will be useful in therapeutic and diagnostic applications in the treatment of heart and other muscular disorders (e.g., anti-arrhythmic agents), supplementation of defective clotting Factor XI in clotting deficiencies, and cobalamin-deficiencies (e.g., pernicious anemia).

MEM6 is homologous to members of the Phosphatase I Protein family of proteins. Thus, the MEM6 nucleic acids and polypeptides, antibodies and related compounds according to the invention will be useful in therapeutic and diagnostic applications in the treatment of diabetes and related disorders originating in dysregulation of glycogen metabolism.

MEM7 and MEM8 are homologous to members of the Retinol-Binding Protein family of proteins. Thus, the MEM7 and MEM8 nucleic acids and polypeptides, antibodies and related compounds according to the invention will be useful in therapeutic and diagnostic applications in the treatment of vision-related disorders (e.g., keratomalacia), and cancer and/or similar neoplastic pathologies.

The MEMX nucleic acids and polypeptides can also be used to screen for molecules, which inhibit or enhance MEMX activity or function. Additional utilities for MEMX nucleic acids and polypeptides according to the invention are disclosed herein.

MEM1

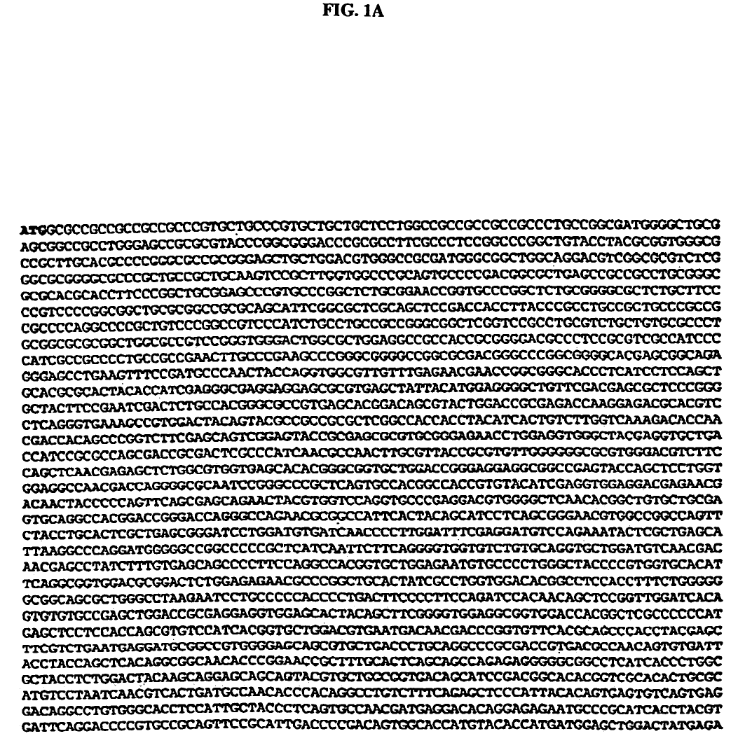

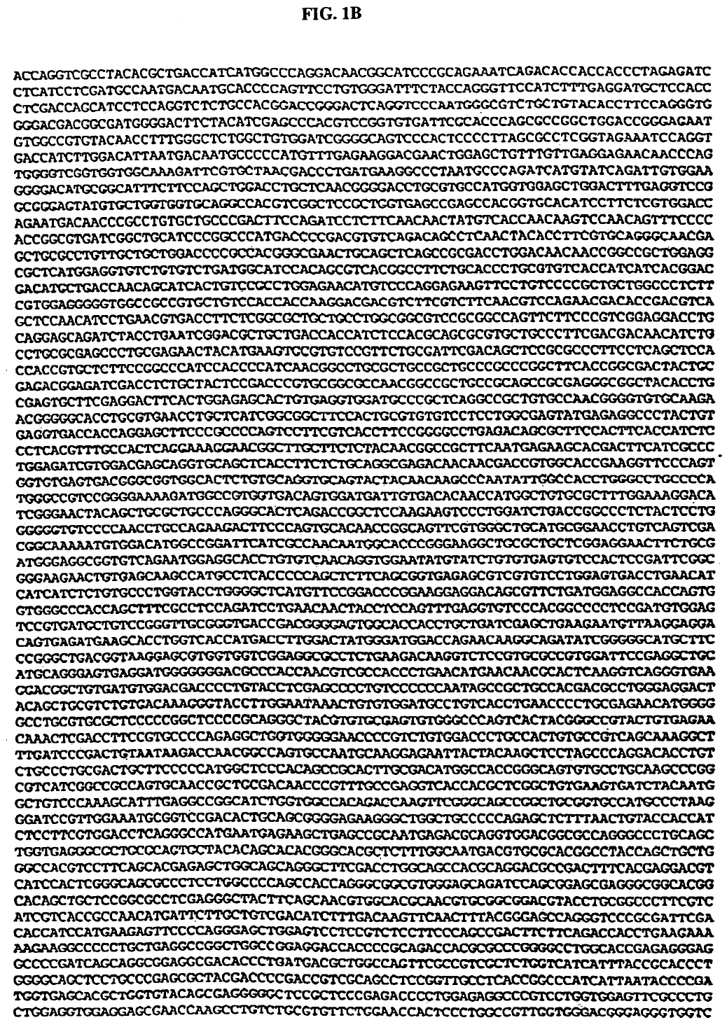

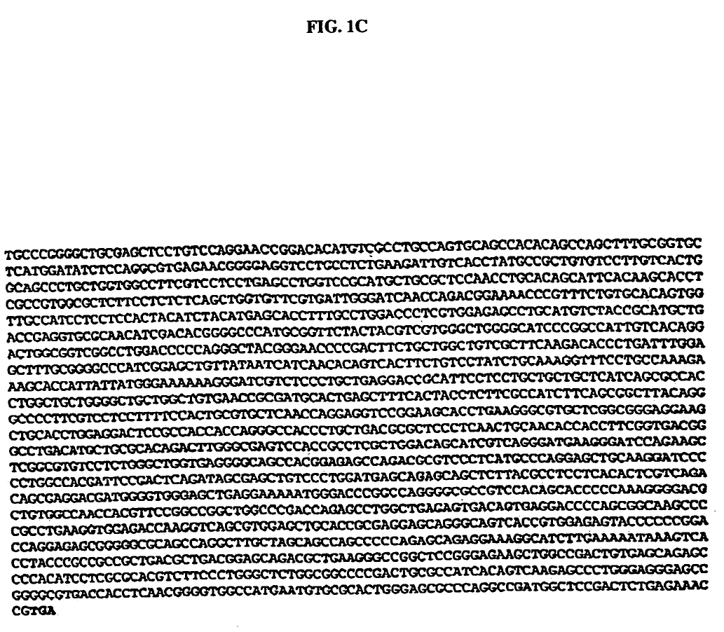

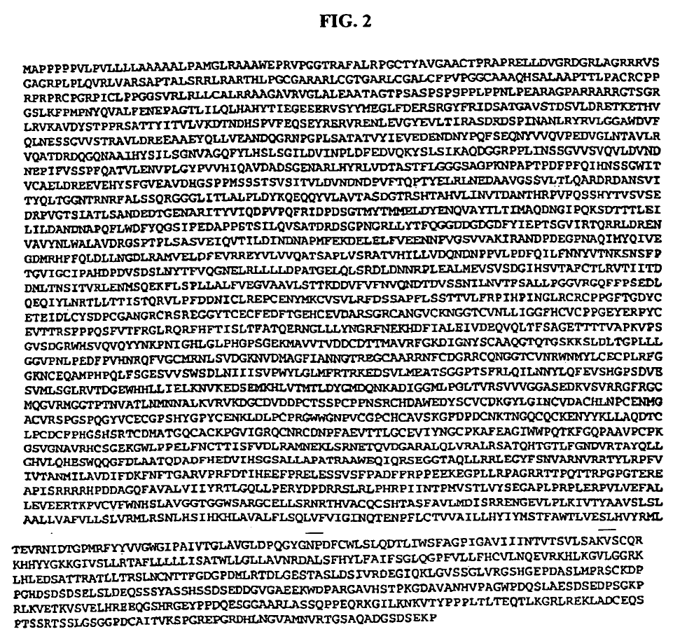

An MEM1 sequence according to the invention iincludes a nucleic acid sequence encoding a polypeptide related to the seven-pass transmembrane receptor family of proteins. The nucleotide sequence [SEQ ID NO:1] of the novel nucleic acid (designated CuraGen Acc. Nos. AL021392, AL031588, and AL031597) encoding a novel protein resembling the seven-pass transmembrane receptor proteins is shown in FIG. 1. An Open Reading Frame (ORF) was identified beginning with an atg initiation codon and ending with a tga termination codon. Putative untranslated regions upstream from the initiation codon and downstream from the termination codon are shown by underlining, and the start and stop codons are shown in bold letters. The amino acid sequence [SEQ ID NO:2] of the encoded protein is presented using the one-letter code in FIG. 2.

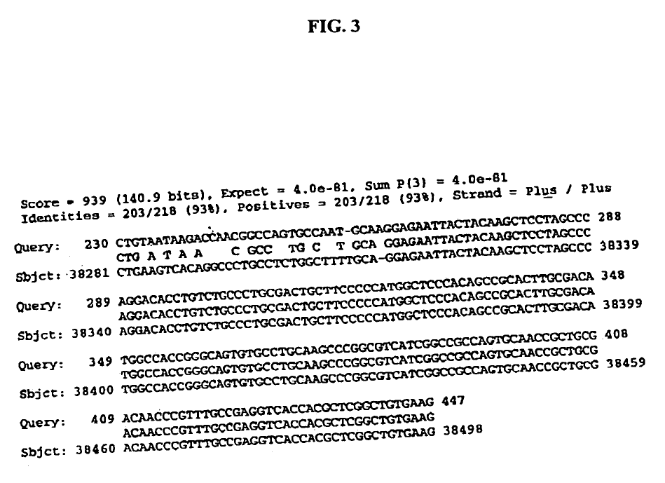

In a BlastN search of nucleic acid sequence databases (see, FIG. 3), it was found, e.g., that the MEM1 nucleic acid sequence has 203 of 218 bases (93%) positive and 203 of 218 bases (93%) identical to sequence (designated HS1163J1) which contains: the 3′ region of a gene for a novel KIAA0279-lile EGF-like domain containing a protein similar to murine Celsr1 and rat MEGF2; a novel gene for a protein similar to C. elegans B0035.16 and bacterial tRNA (5′-Methylaminomethyl-2-thiouridylate)-Methyltransferases; and the 3′ region of a novel gene for a protein similar to murine B99.

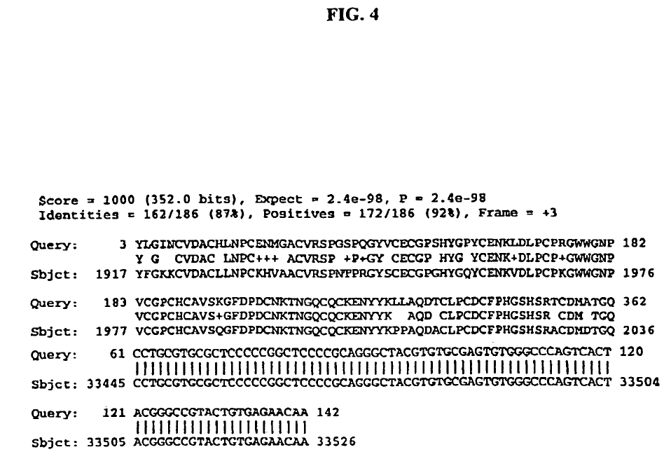

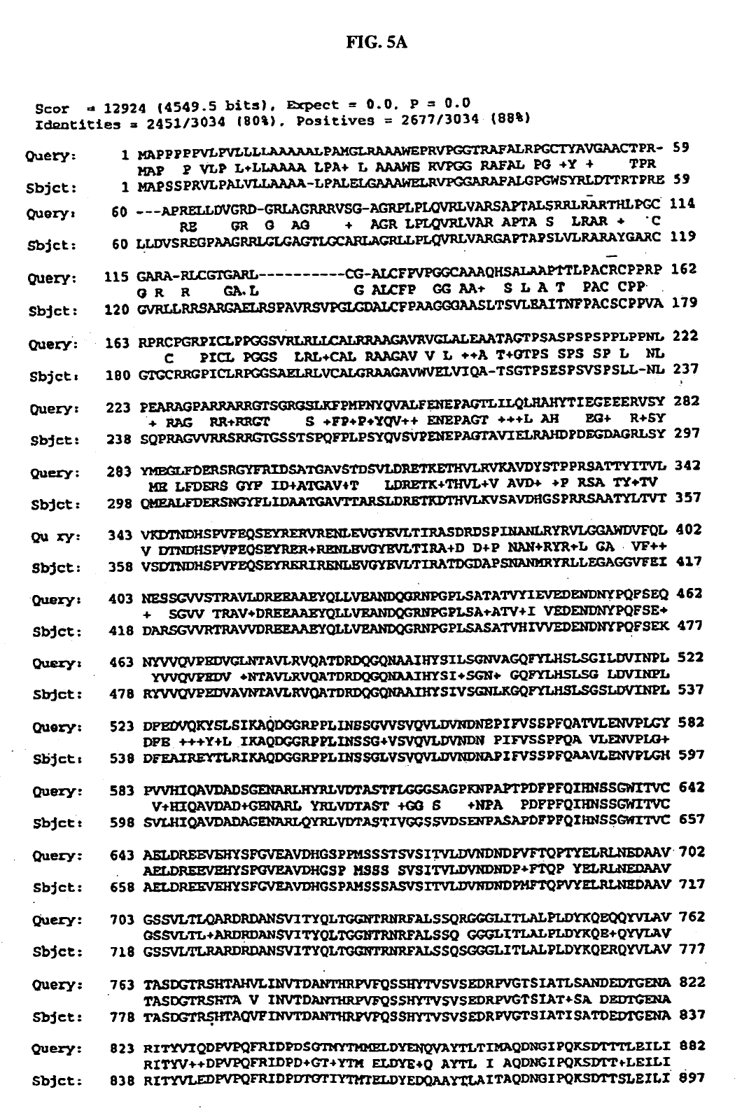

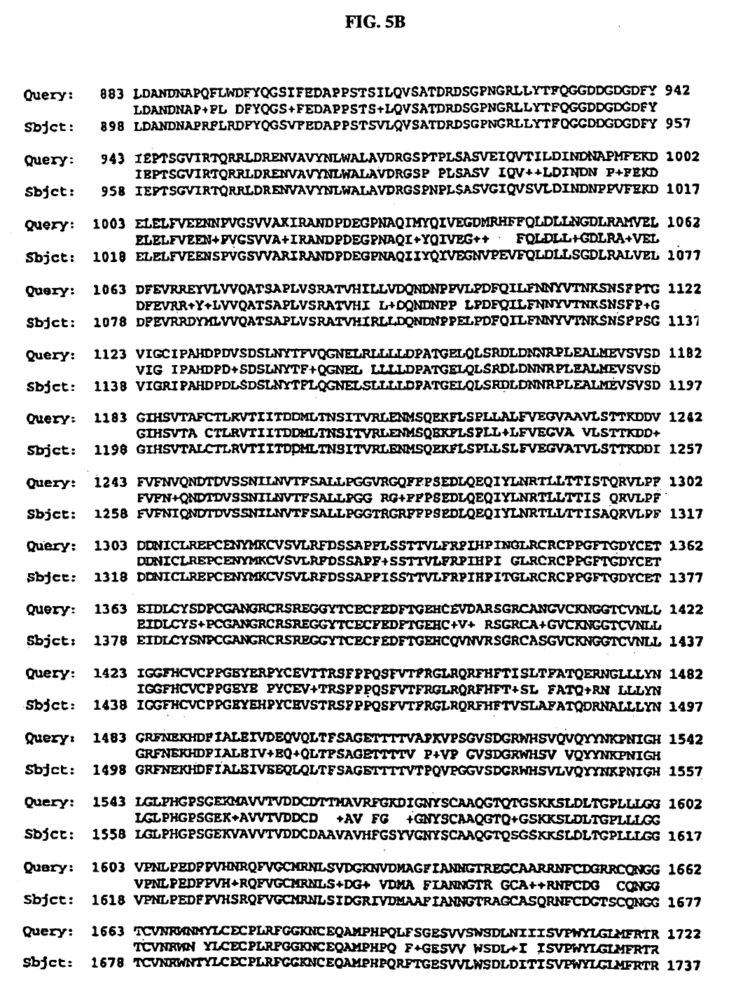

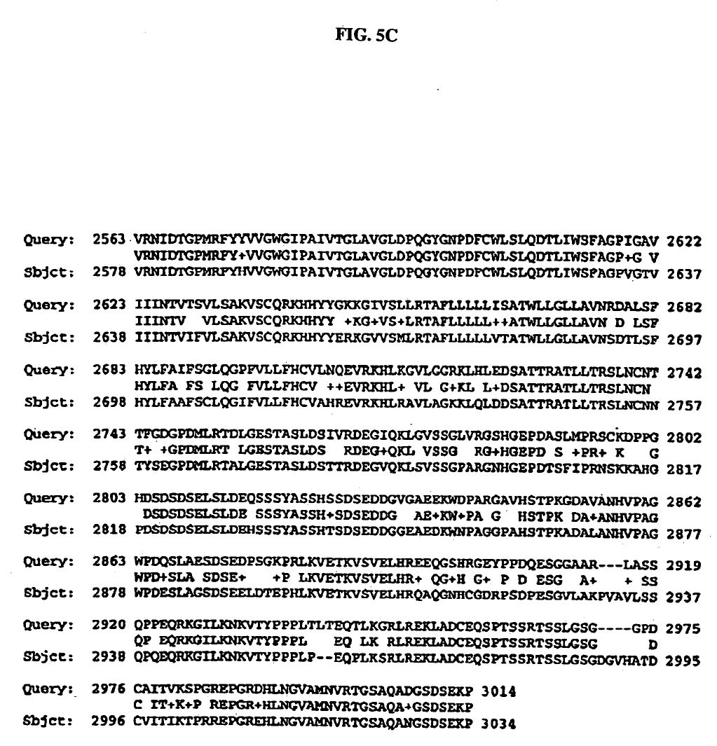

In a search of amino acid databases, the MEM1 protein of the invention was found to have 172 of 186 amino acid residues (91%) positive with, and 162 of 186 amino acid residues (87%) identical to the seven-pass transmembrane receptor protein precusor MouseA (ptnr: PIR-ID:T14119, see, FIG. 4) which is a member of the Celsr family of seven-pass transmembrane receptor proteins which are expressed during embryogenesis in the mouse. In a BlastP search (see, FIG. 5), the protein of the present invention was found to have 2345 of 2632 amino acid residues (89%) positive with, and 2139 of 2632 amino acid residues (81%) identical to the amino acid residue seven-pass transmembrane receptor protein precusor MouseA.

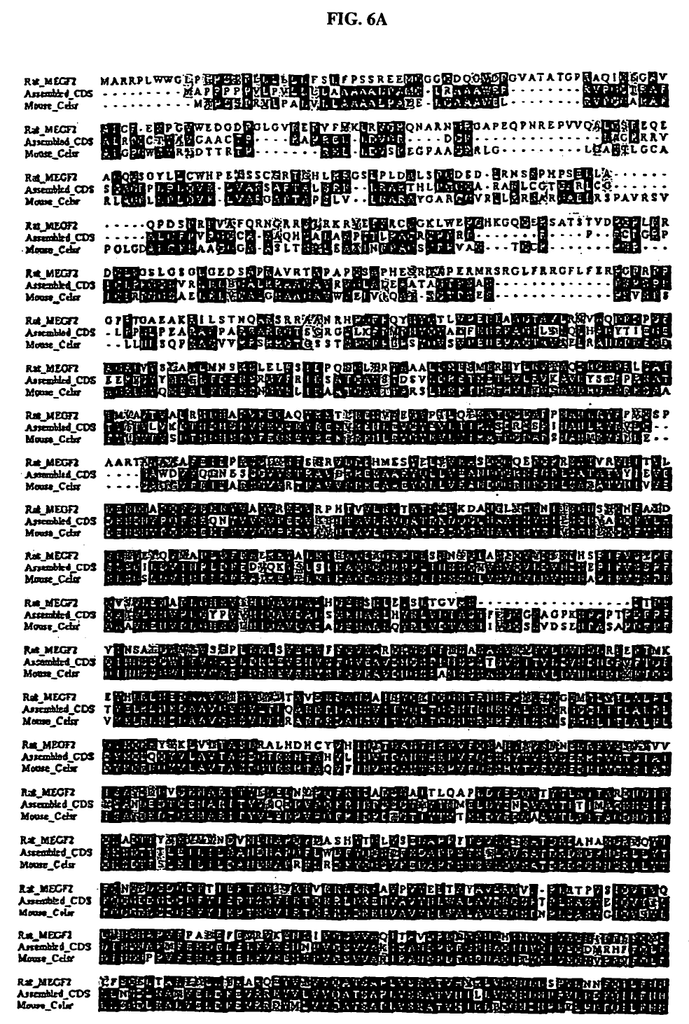

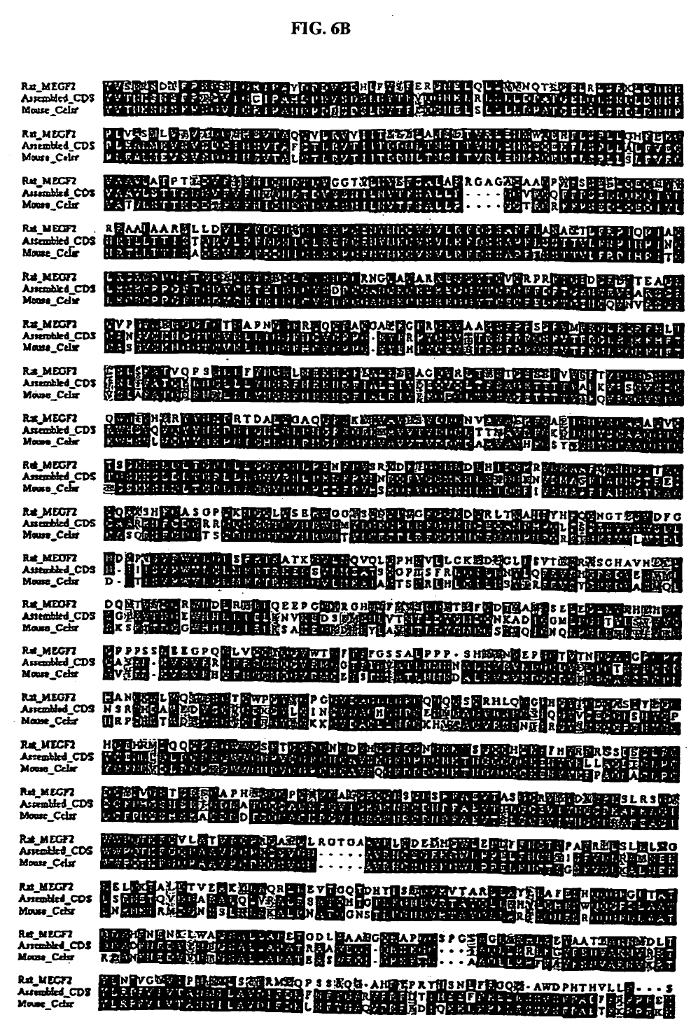

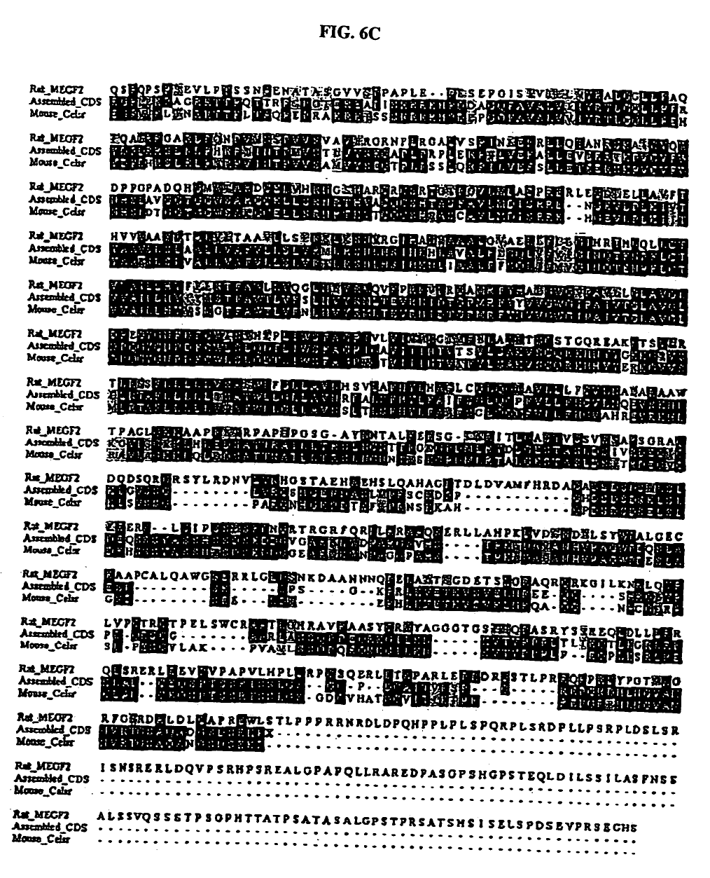

A multiple sequence alignment is illustrated in FIG. 6, with the protein of the invention being shown on Line 2, in a ClustalW analysis comparing the protein of the invention with related protein sequences.

The novel nucleic acid of the invention encoding a protein resembling the seven-pass transmembrane receptor family of proteins includes the nucleic acid whose sequence [SEQ ID NO:1] is provided in FIG. 1, or a fragment thereof. The invention also includes a mutant or variant nucleic acid any of whose bases may be changed from the corresponding base shown in FIG. 1, while still encoding a protein that maintains its retinol-binding activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to 20% or more of the bases may be so changed.

The novel protein of the invention includes the proteins resembling seven-pass transmembrane receptor proteins whose sequence [SEQ ID NO:2] is provided in FIG. 2. The invention also includes a mutant or variant protein any of whose residues may be changed from the corresponding residue shown in FIG. 2, while still encoding a protein that maintains its proteins resembling retinol-binding activities and physiological functions, or a functional fragment thereof. In the mutant or variant protein, up to 20% or more of the residues may be so changed. The invention further encompasses antibodies and antibody fragments, such as Fab or (Fab)₂, that bind immunospecifically to any of the proteins of the invention.

MEM2, MEM3, and MEM4

An MEM2, MEM3, and MEM4 sequence according to the invention includes a nucleic acid sequence encoding a polypeptide related to the human glutamate receptor family of proteins. Three variants of a human glutamate receptor MEM2 (Internal identification No. 21659259 EXT 1); MEM3 (Internal identification No. 21659259 EXT 2); and MEM4 (Internal identification No. 21659259 EXT 3) are disclosed in the present invention. These differing sequences apparently result from splice variants (or a similar deletion) at the nucleic acid level and resemble a lung-specific, splice-form of a previously reported glutamate receptor (SPTREMBL-ACC:O60391). Each of the three splice variants will be discussed below.

Splice Variant 21659259 EXT 1 (MEM2)

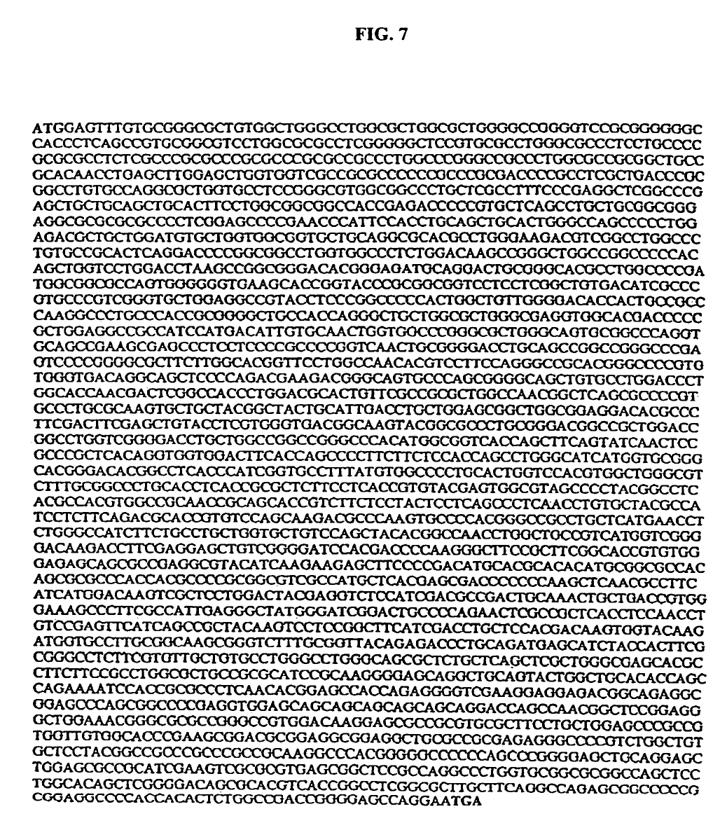

The nucleotide sequence of one splice variant of the present invention MEM2 (Internal Identification No. 21659259 EXT 1) is shown in FIG. 7 [SEQ ID NO:3]. An Open Reading Frame (ORF) was identified beginning with the atg initiation codon and ending with the tga termination codon. The start and termination codons are shown in bold letters. The encoded protein is illustrated using one-letter amino acid code in FIG. 8 [SEQ ID NO:4].

In this splice variant, the difference was found at amino acid residue 360, where 73 amino acids residues were shown to be deleted (i.e., “spliced-out”). These amino acid residues are also present in the best Blast-X protein match (SPTREMBL-ACC:O60391). It is important to note that these 73 amino acids are also spliced out in a reported glutamate receptor from human brain (SWISSPROT-ACC:Q14957). Therefore, this variant may represent an isoform of a glutamate receptor that is present in both lung and brain.

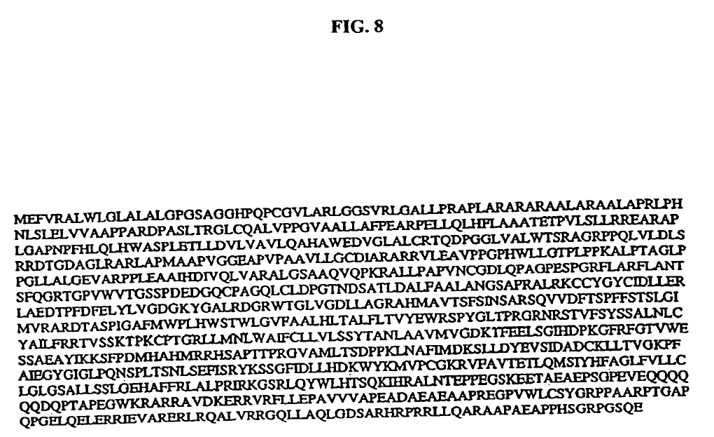

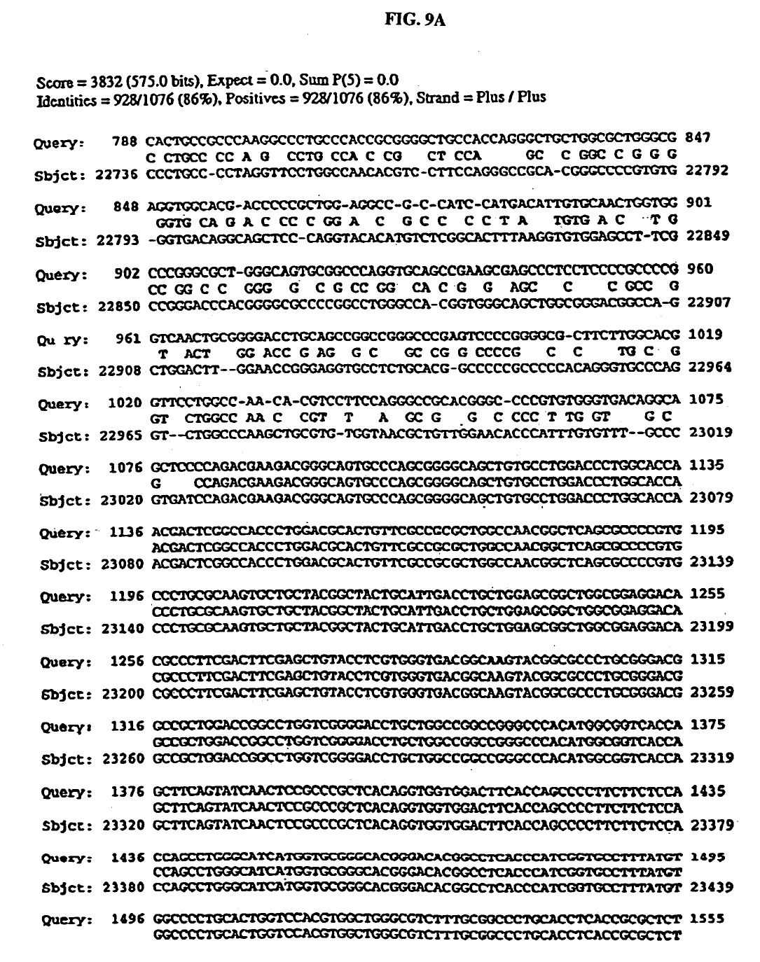

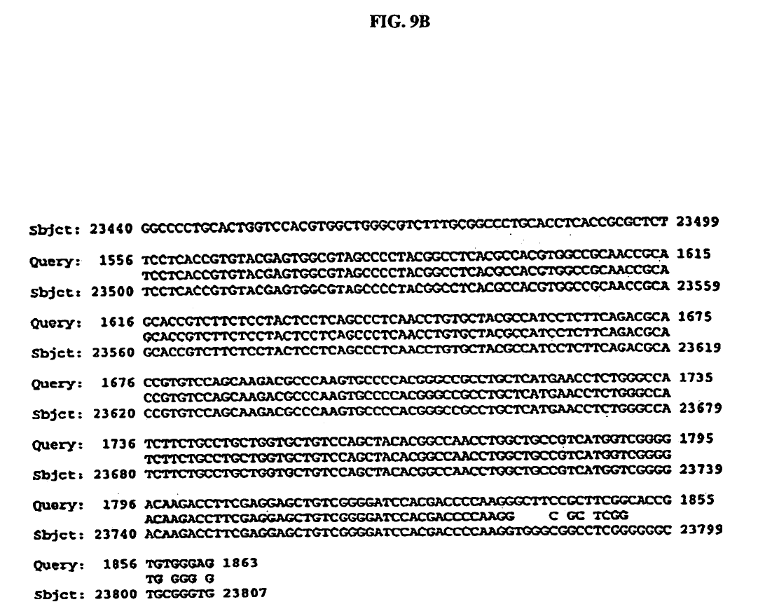

BLASTN comparisons leading to the assembly of the 21659259 EXT 1 variant of this invention are illustrated in FIG. 9. As noted above, the sequences of the present invention match a genomic sequence (SPTREMBL-ACC:O60391). In assembling and verifying the sequences, one correction was made to the SeqCalling assembly, which added a G at nucleotide 104 of the assembly. It was noted that the sequencing trace appearance also suggested that another G could be present in the sequence at basepair 104. Furthermore, adding the G corrected a frame shift in the protein and resulted in a better Blast-X match with other reported glutamate receptors. This gene, 21659259 EXT 1, differs from the previously reported gene (SPTREMBL-ACC:O60391). The protein in the public database (SPTREMBL-ACC:060391) includes 73 amino acids that are missing in the present 21659259 EXT 1 sequence. It is believed that the presently disclosed assembly (21659259 EXT 1), which is derived from fetal lung tissue, represents a splice variant of the reported protein. This represents omission of bases 22806-23025 of the genomic sequence (GENBANK-ID:AC004528).

The protein in the public database (SPTREMBL-ACC:O60391) additionally includes 6 amino acid residues at the beginning of the exon (i.e., basepairs 25855-26000) of the genomic sequence (GENBANK-ID:AC004528). In the presently disclosed sequence, however, the same exon includes only the region between basepairs 25873-26000 bp, and does not contain the 18 nucleotides which lie between basepairs 25855-25873 of the genomic sequence. Accordingly, the protein variant 21659259 EXT 1 of the present invention lacks the six amino acids, present in the human and rat reference sequences, encoded by these missing bases.

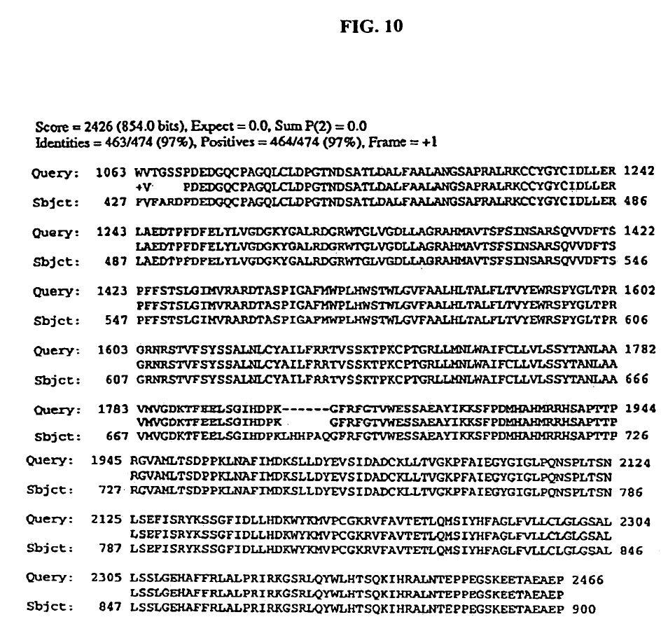

Additionally, the protein found in the public database (SPTREMBL-ACC:O60391) also lacks the last exon containing 430 bp predicted by GenScan in the present invention. This exon terminates with the stop codon TGA. BLASTX comparisons used in identifying variant 21659259 EXT 1 are shown in FIG. 10.

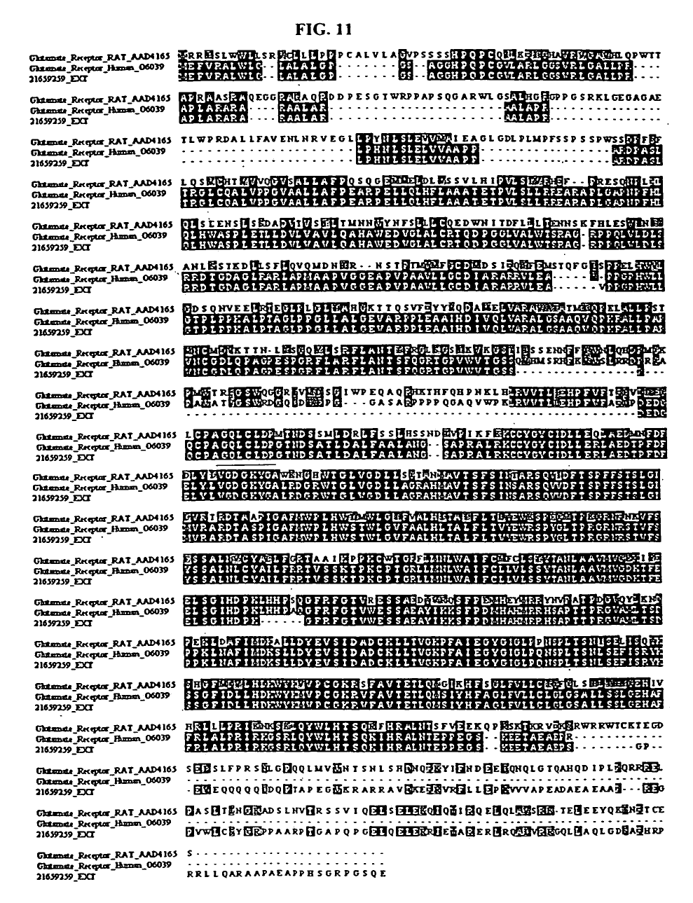

A multiple sequence alignment of variant 21659259 EXT 1 is illustrated in FIG. 11, with the protein of the invention being shown on Line 3, in a ClustalW analysis comparing the protein of the invention with related protein sequences. The 73-residue and 6-residue deletions are shown, as is the C-terminal extension.

Splice Variant 21659259 EXT 2 (MEM3)

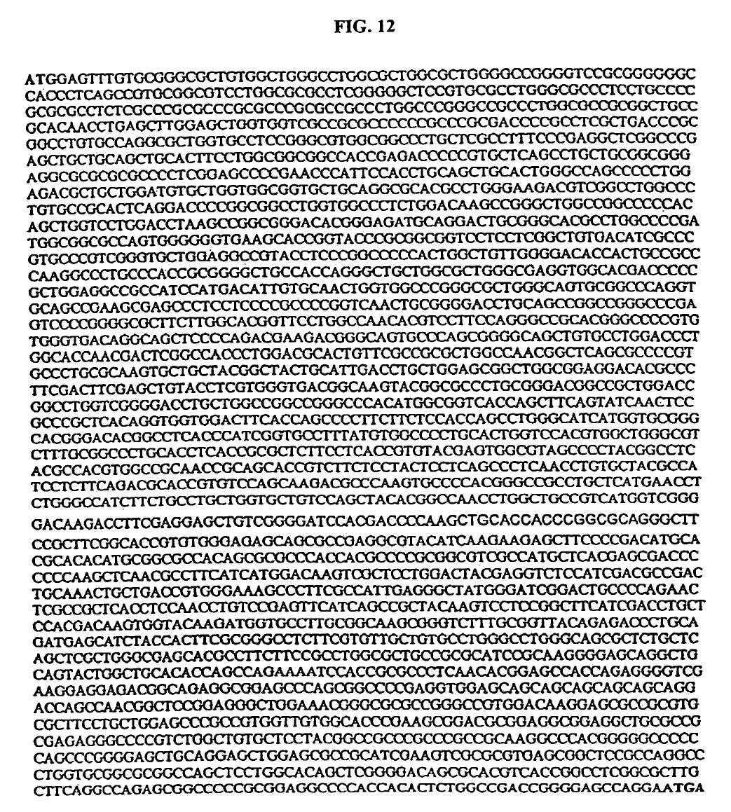

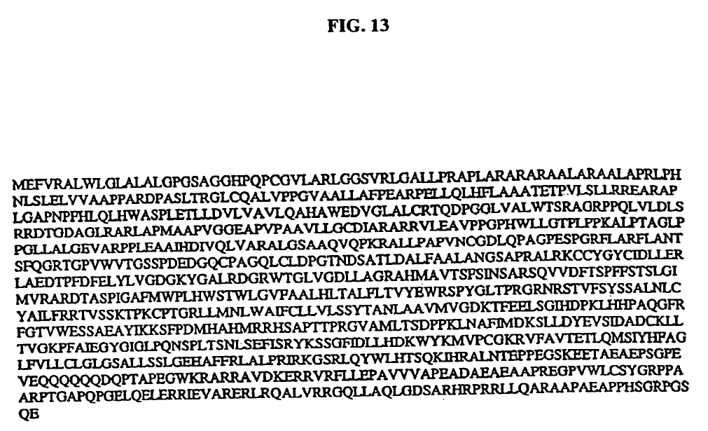

The nucleotide sequence [SEQ ID NO:5] of a second splice variant, MEM3 (Internal Identification No. 21659259 EXT 2), of the present invention is shown in FIG. 12. An Open Reading Frame (ORF) was identified beginning with an atg initiation codon and ending with a tga termination codon. The start and termination codons are shown in bold letters. The encoded protein [SEQ ID NO:6] is illustrated using the one-letter amino acid code in FIG. 13.

Two of the three distinctions found in MEM2 (the 21659259 EXT 1 variant) were also demonstrated to be present with this splice variant. However, it was believed that the eighteen nucleotide omission noted for MEM2 (21659259 EXT 1) should be included in view of the fact that this fragment is present in a variety of glutamate receptors. Thus the amino acids encoded by these nucleotides are included in the amino acid sequence of this variant.

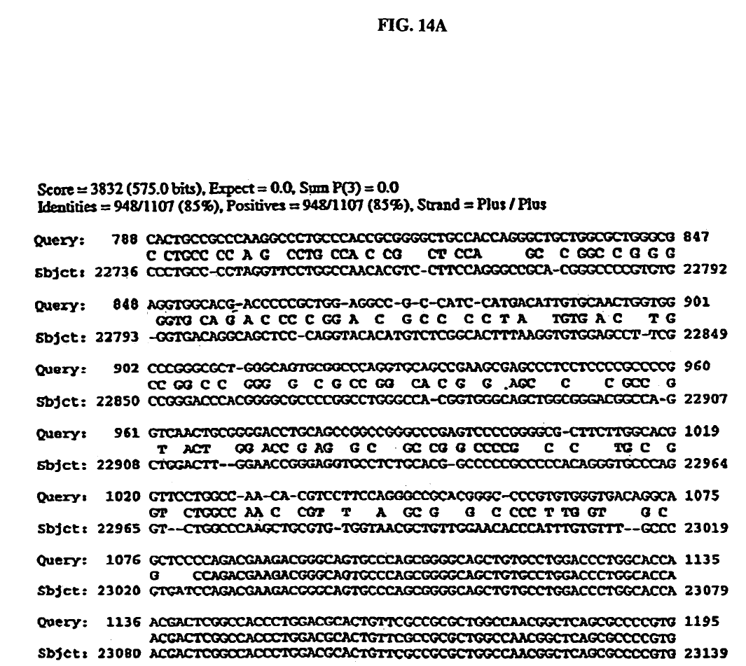

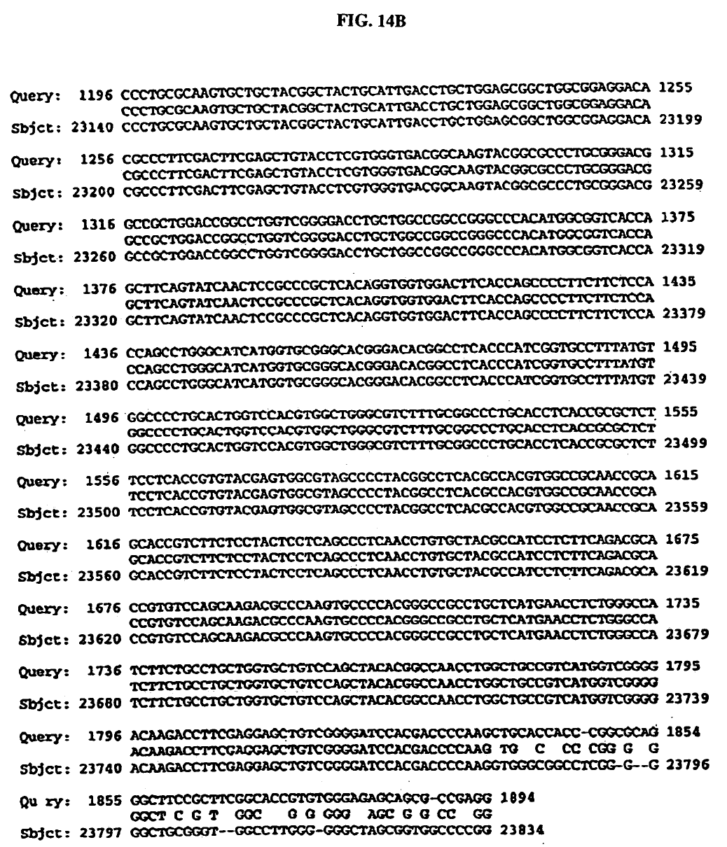

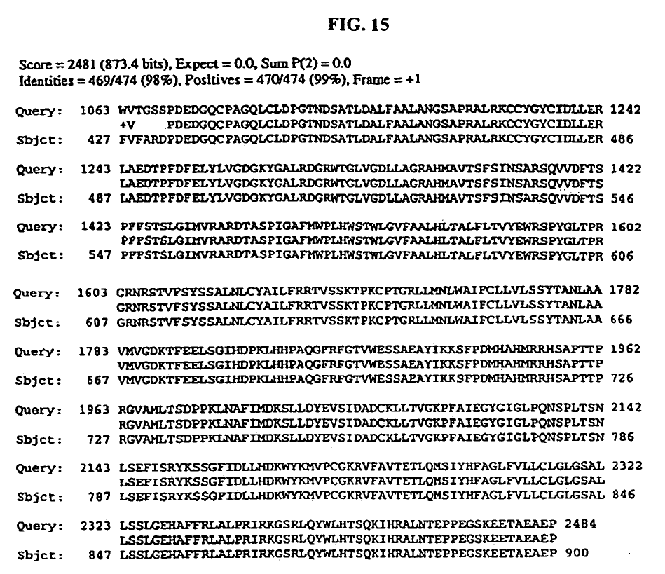

BLASTN comparisons leading to the assembly of the 21659259 EXT 2 variant of this invention are included in FIG. 14. BLASTX comparisons used in identifying variant 21659259 EXT 2 are shown in FIG. 15.

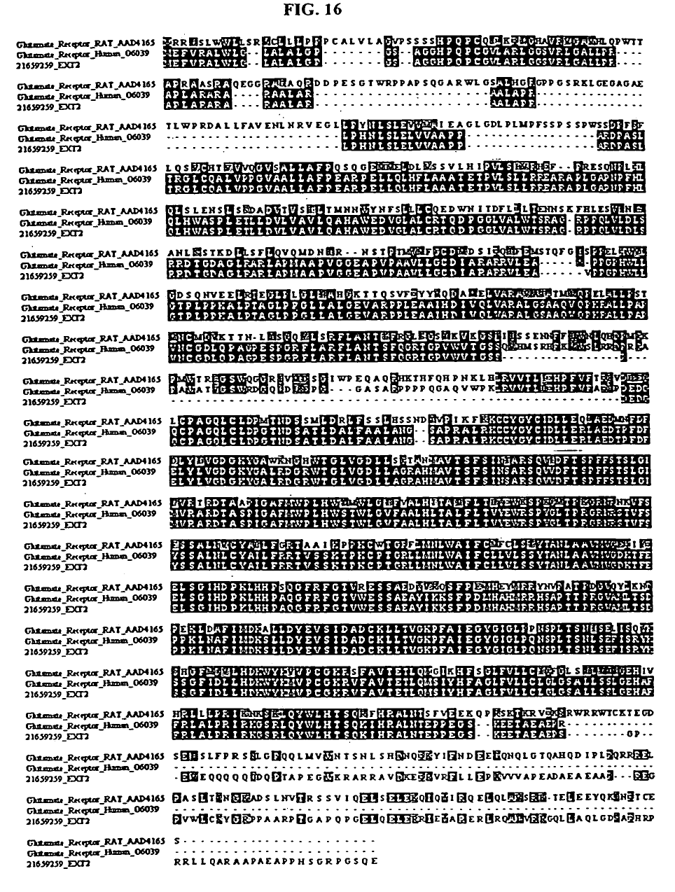

A multiple sequence alignment is of MEM3 variant 21659259 EXT 2 given in FIG. 16, with the protein of the invention being shown on Line 3, in a ClustalW analysis comparing the protein of the invention with related protein sequences. The 73-residue deletion is shown, as is the carboxyl-terminal extension.

Splice Variant 21659259 EXT 3 (MEM4)

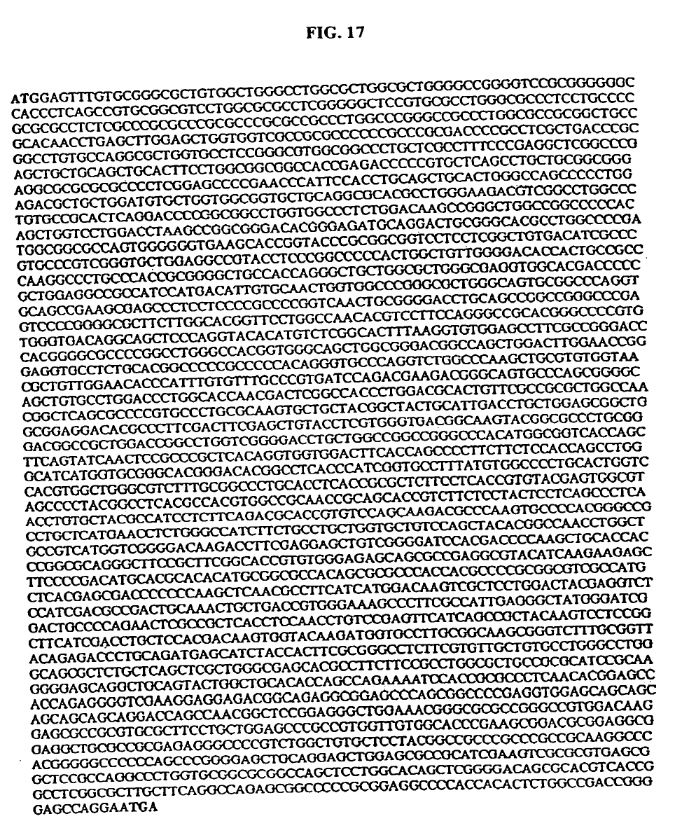

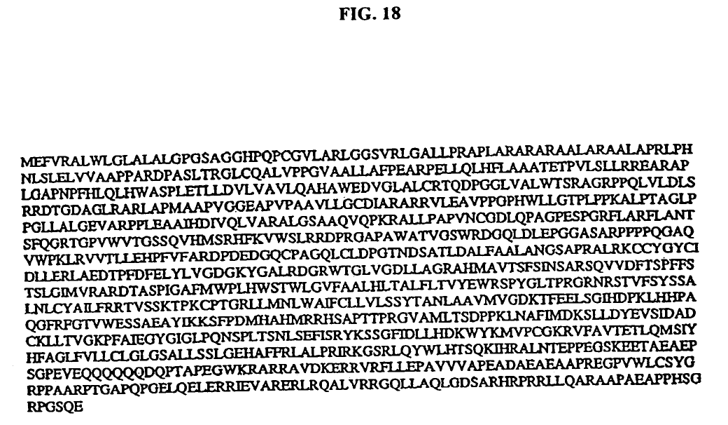

The nucleotide sequence [SEQ ID NO:7] of a third splice variant, MEM4 (Internal Identification No. 21659259 EXT 3), of the invention is shown in the nucleotide sequence of FIG. 17. An open reading frame was identified beginning with an atg initiation codon and ending with a tga termination codon. The start and stop codons are in bold letters. The amino acid sequence [SEQ ID NO:8] of the encoded protein is presented using the one-letter code in FIG. 18.

One of the three distinctions found with MEM2 (21659259 EXT 1) also occur in this variant. Due to the fact that these fragments have been shown to be present in a variety of glutamate receptors, both the eighteen nucleotide omission noted for MEM2 (21659259 EXT 1), as well as the 73 amino acid deletion, were included in the sequence of this splice variant. Thus, the amino acid sequences represented by these deletions are included in the amino acid sequence of this variant.

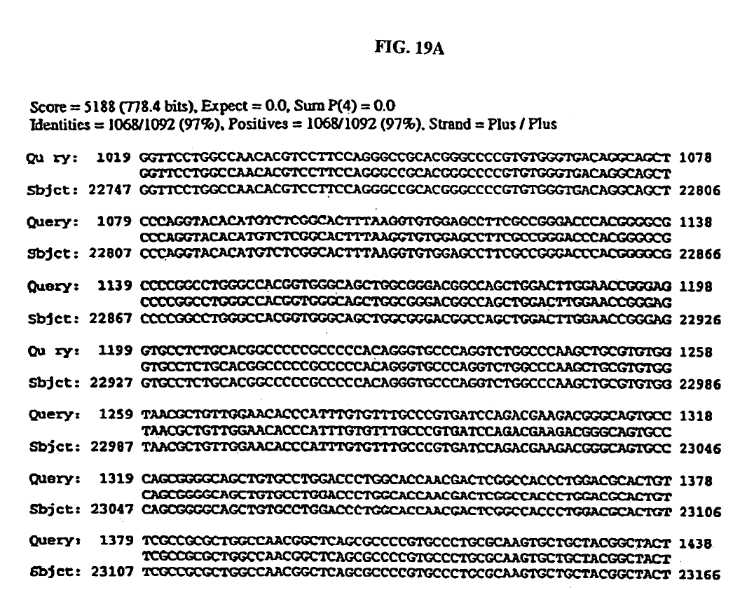

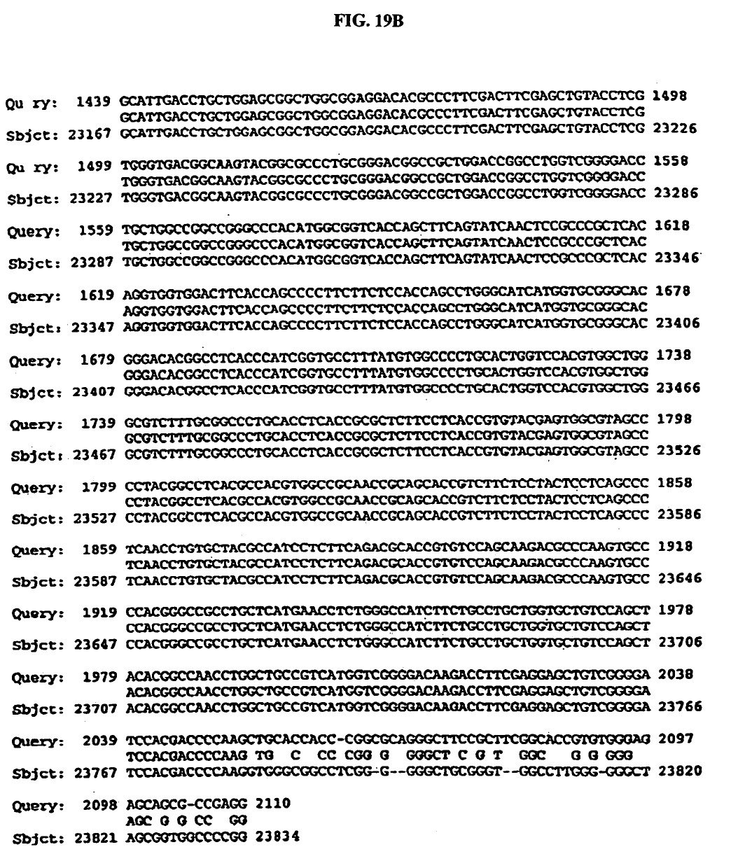

BLASTN comparisons leading to the assembly of MEM4 are illustrated in FIG. 19; whereas the BLASTX comparisons used in identifying MEM4 are illustrated FIG. 20. Although the match for 900 of the 901 residues of the SPTREMBL-ACC:O60391 sequence is 100% identical to that of 21659259 EXT 3, the public protein (SPTREMBL-ACC:O60391) is found to lack the terminal 143 amino acids included in the splice variants of the present invention.

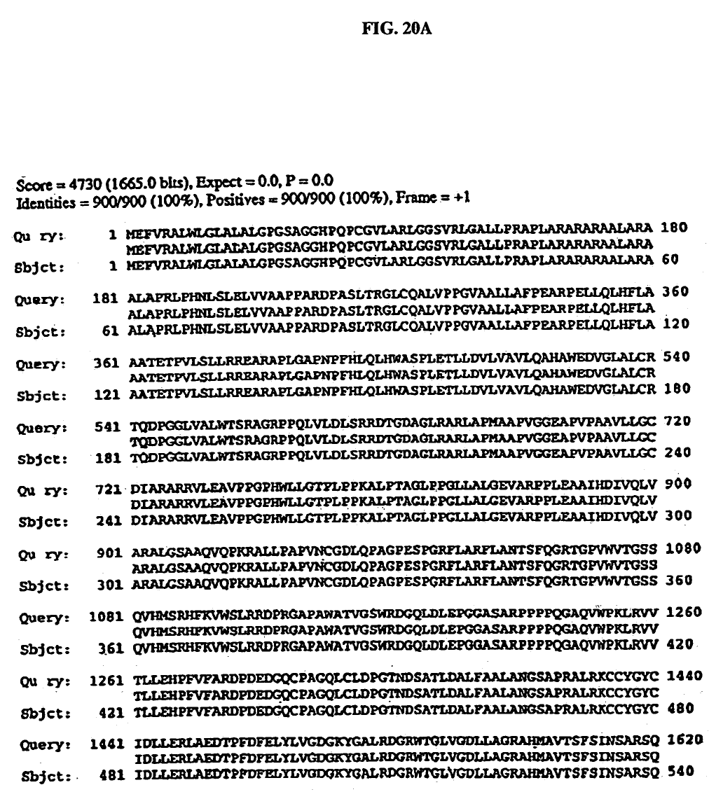

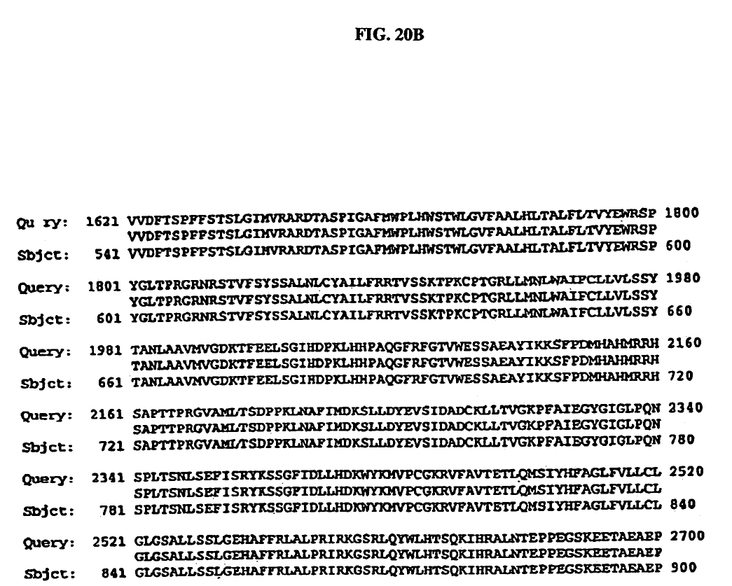

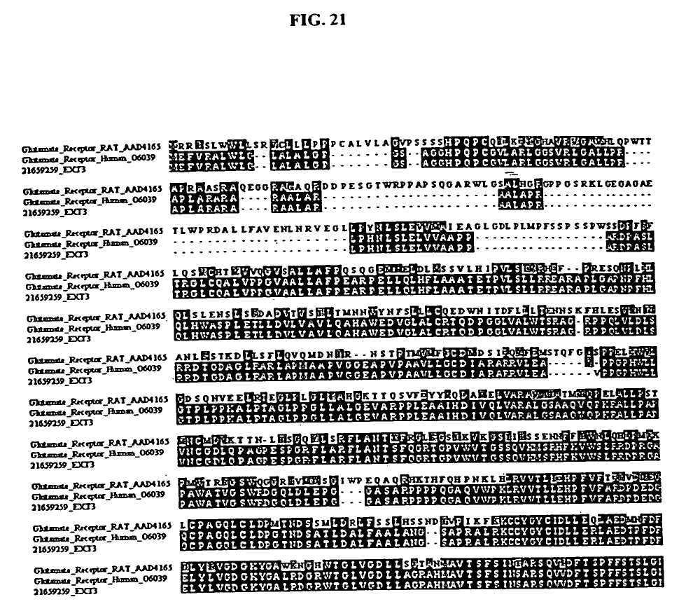

ClustalW analysis comparing variant 21659259 EXT 3 with related protein sequences is illustrated in FIG. 21, with the protein of the invention being shown on Line 3. In addition, the carboxyl-terminal extension is shown.

A comparative alignment of the three splice variants of the present invention MEM2, MEM3, and MEM4 (i.e., 21659259 EXT 1; 21659259 EXT 2; and 21659259 EXT 3) is shown in FIG. 22.

The novel nucleic acid of the invention encoding a glutamate receptor includes the nucleic acid whose sequence is provided in FIG. 7 [SEQ ID NO:3]; FIG. 12 [SEQ ID NO:5]; and FIG. 17 [SEQ ID NO:7], or fragments thereof. The present invention also includes a mutant or variant nucleic acid any of whose bases may be changed from the corresponding base shown in FIGS. 7, 12, and 17, while still encoding a protein that maintains its glutamate receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to 20% or more of the bases may be so changed.

The novel protein of the invention includes the following proteins: FIG. 8 [SEQ ID NO:4]; FIG. 13 [SEQ ID NO:6]; and FIG. 18 [SEQ ID NO:8]. The invention also includes a mutant or variant protein any of whose residues may be changed from the corresponding residue shown in FIG. 8, FIG. 13, and FIG. 18, while still encoding a protein that maintains its glutamate receptor-like protein-like activities and physiological functions, or a functional fragment thereof. In the mutant or variant protein, up to 20% or more of the residues may be so changed. The invention further encompasses antibodies and antibody fragments, such as Fab or (Fab)₂, that bind immunospecifically to any of the proteins of the invention.

MEM5

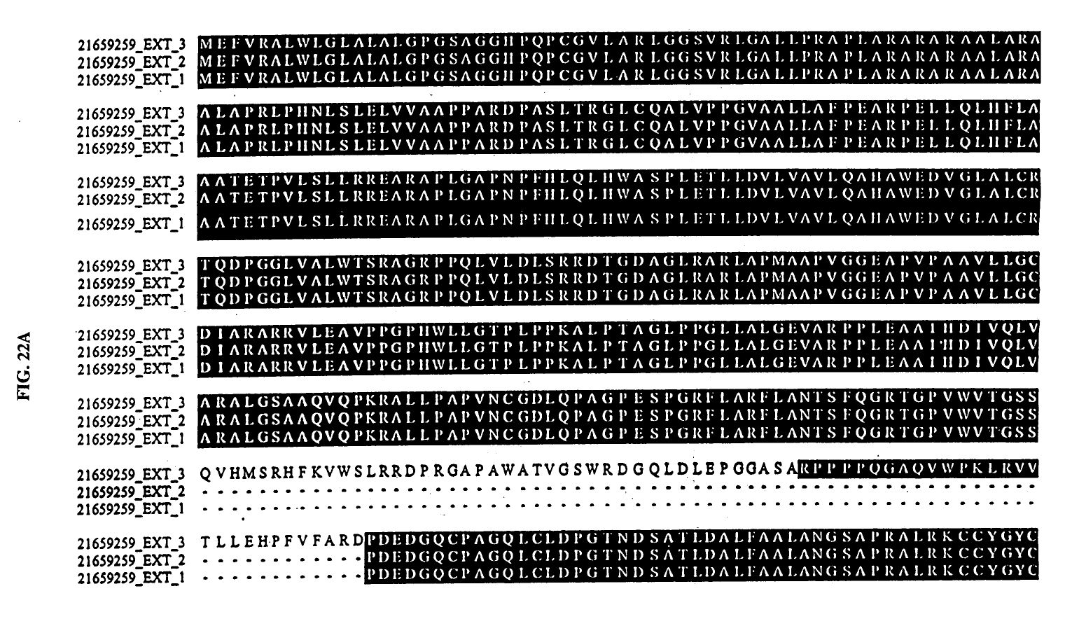

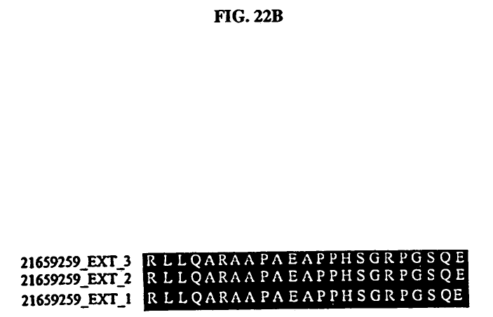

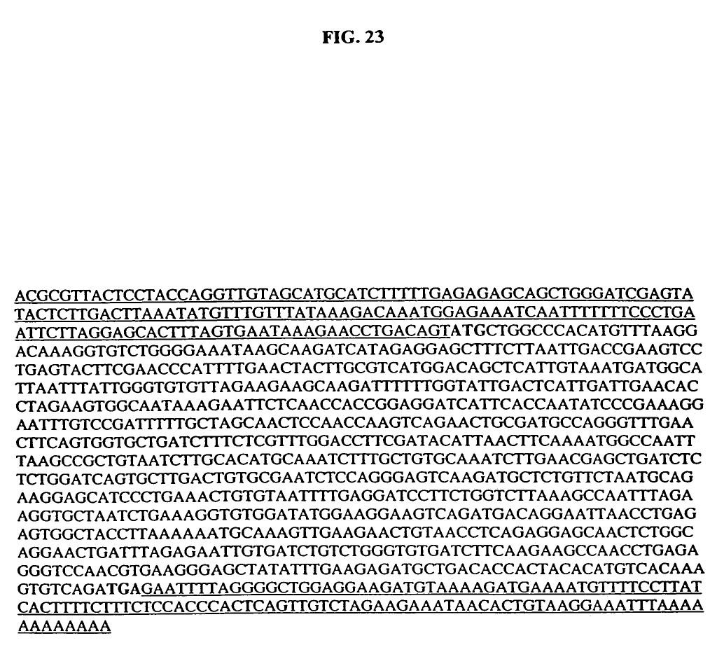

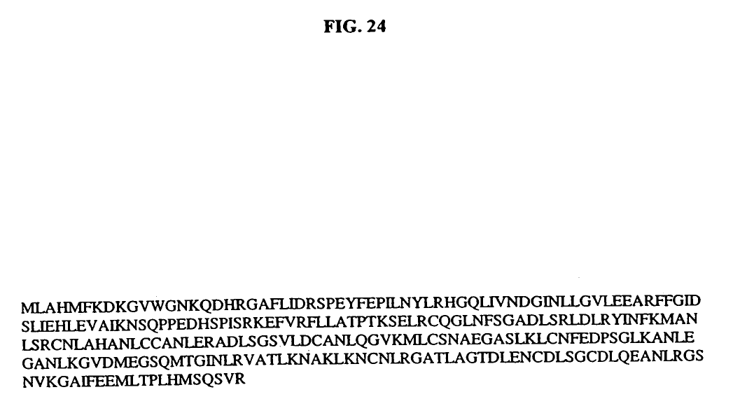

An MEM5 sequence according to the invention includes a nucleic acid sequence encoding a polypeptide related to the potassium channel proteins. The novel nucleic acid sequence [SEQ ID NO:9] of 1110 nucleotides (Internal Identification No. 16418841_EXT) encoding a ion channel-like protein is shown in FIG. 23. An Open Reading Frame (ORF) of 828 nucleotides was identified beginning with an atg initiation codon and ending with a tga termination codon (see, FIG. 23; [SEQ ID NO:9]). Putative untranslated regions, one upstream from the initiation codon and another downstream of the termination codon, are shown by underlining in FIG. 23, whereas the start and termination codons are shown in bold letters. The sequence of the encoded protein [SEQ ID NO:10] comprising 275 amino acid residues is presented using the one-letter amino code in FIG. 24.

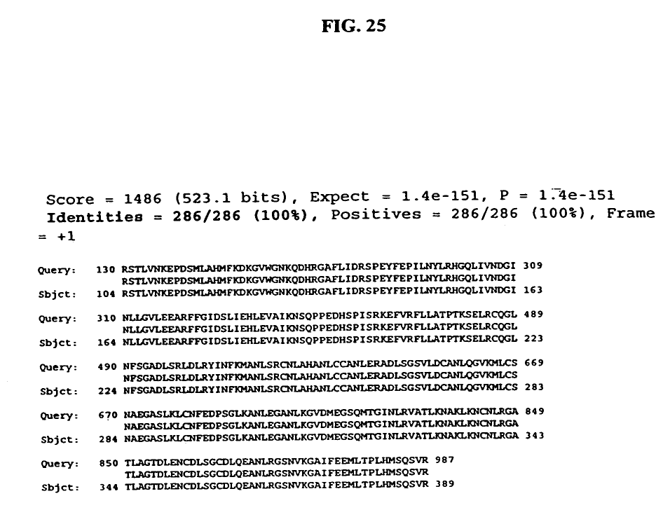

In a search of sequence databases (see, FIG. 25), it was found, e.g., that the nucleic acid sequence of the protein of the invention has found to have 286 of 286 amino acid residues (100%) identical to, and 286 of 286 amino acid residues (100%) positive with, the Human potassium channel protein K⁺Hnov42 (patp:Y34130; see, International Publication No. WO 9943696 A1).

A hydrophobicity plot shows that the protein of the invention has a short, N-terminal, hydrophilic sequence (140 aa), followed by a hydrophobic region (41-65 aa, peak hydrophobicity=1), followed by a hydrophilic C-terminus. Although a SignalP analysis suggests that there is no signal peptide, the hydrophobic region at 41-65 may nevertheless be a cleavable signal peptide.

The novel nucleic acid of the invention includes the nucleic acid whose sequence [SEQ ID NO:9] is provided in FIG. 23, or a fragment thereof. The invention also includes a mutant or variant nucleic acid any of whose bases may be changed from the corresponding base shown in FIG. 23, while still encoding a protein that maintains its activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to 20% or more of the bases may be so changed.

The novel protein of the invention includes the protein whose sequence [SEQ ID NO:10] is provided in FIG. 24. The invention also includes a mutant or variant protein any of whose residues may be changed from the corresponding residue shown in FIG. 24, while still encoding a protein that maintains its potassium channel protein-like activities and physiological functions, or a functional fragment thereof. In the mutant or variant protein, up to 20% or more of the residues may be so changed. The invention further encompasses antibodies and antibody fragments, such as Fab or (Fab)₂, that bind immunospecifically to any of the proteins of the invention.

MEM6

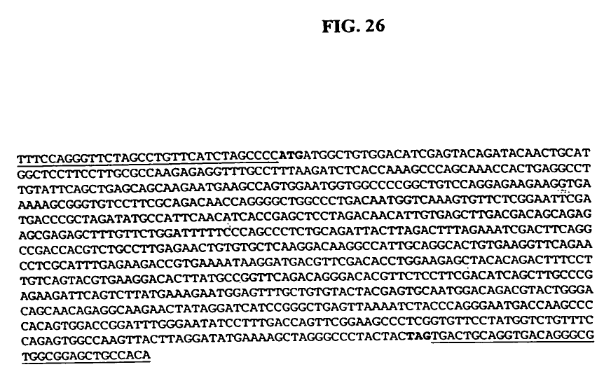

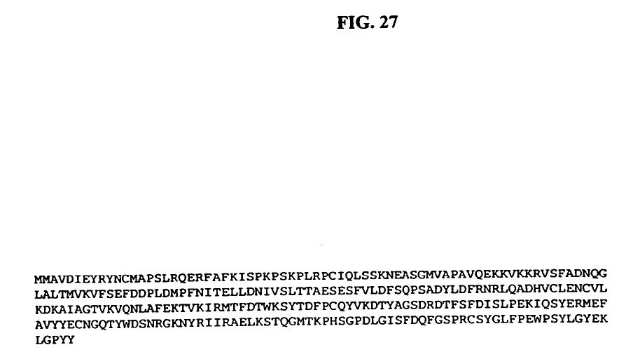

An MEM6 sequence according to the invention includes a nucleic acid sequence encoding a polypeptide related to the glycogen-binding, phosphatase 1 protein family. The nucleotide sequence [SEQ ID NO:11] of the novel nucleic acid (Internal Identification No. AC016485_A) encoding a glycogen-binding protein phosphatase 1-like protein is shown in FIG. 26. An Open Reading Frame (ORF) was identified beginning with an atg initiation codon and ending with a tag termination codon. Putative untranslated regions upstream from the initiation codon and downstream from the termination codon are shown by underlining, and the start and stop codons are shown in bold letters. The amino acid sequence [SEQ ID NO:12] of the encoded protein is presented using the one-letter code in FIG. 27.

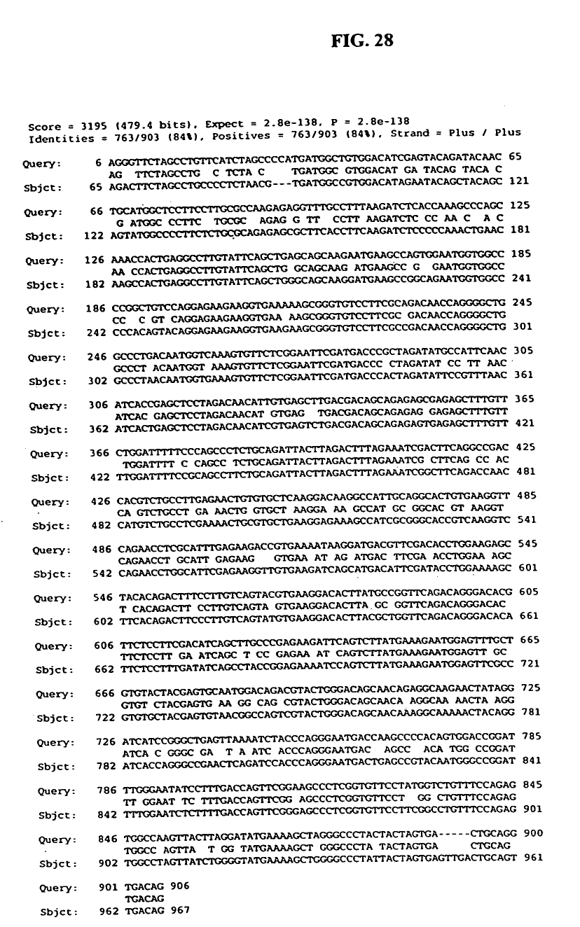

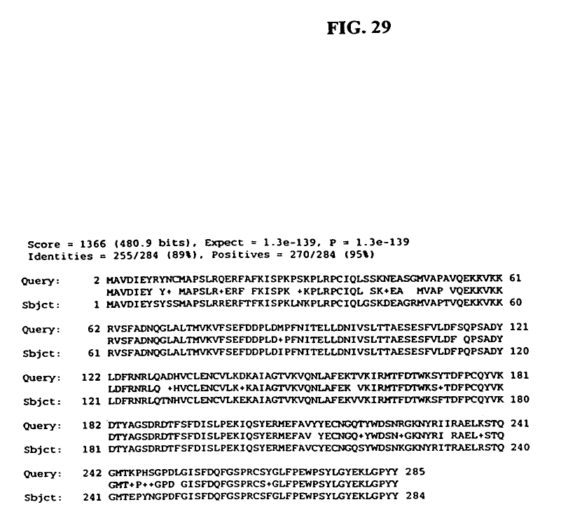

In a search of sequence databases, it was found, for example, that the nucleic acid sequence [SEQ ID NO:11] has 763 of 903 bases (84%) identical to a rat mRNA for protein phosphatase 1 (GL-subunit) (GENBANK-ID:Y18208; see, FIG. 28). The amino acid sequence [SEQ ID NO:12] of the protein of the invention was found to have 255 of 284 amino acid residues (89%) identical to, and 270 of 284 residues (92%) positive with, the 284 amino acid residue hepatic glycogen-binding subunit protein phosphatase-1 from rat (ACC: Q63759; see, FIG. 29).

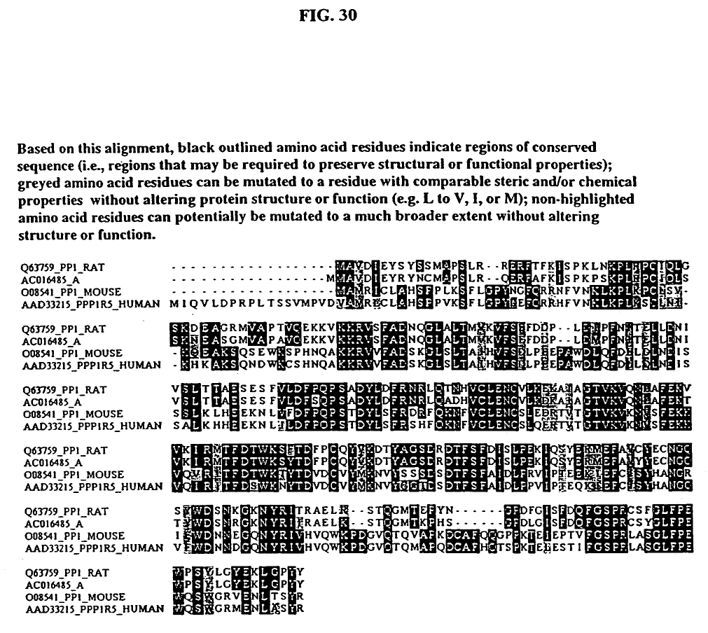

A multiple sequence alignment is illustrated in FIG. 30, with the protein of the invention being shown on Line 2, in a ClustalW analysis comparing the protein of the invention with related protein sequences.

The novel nucleic acid of the invention encoding a glycogen-binding protein phosphatase 1 includes the nucleic acid whose sequence [SEQ ID NO:11] is provided in FIG. 26, or a fragment thereof. The invention also includes a mutant or variant nucleic acid any of whose bases may be changed from the corresponding base shown in FIG. 26, while still encoding a protein that maintains its glycogen-binding protein phosphatase 1-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to 20% or more of the bases may be so changed.

The novel protein of the invention includes the glycogen-binding protein phosphatase 1-like protein whose sequence [SEQ ID NO:12] is provided in FIG. 27. The invention also includes a mutant or variant protein any of whose residues may be changed from the corresponding residue shown in FIG. 27, while still encoding a protein that maintains its glycogen binding protein phosphatase 1-like activities and physiological functions, or a functional fragment thereof. In the mutant or variant protein, up to 20% or more of the residues may be so changed. The invention further encompasses antibodies and antibody fragments, such as Fab or (Fab)₂, that bind immunospecifically to any of the proteins of the invention.

MEM7

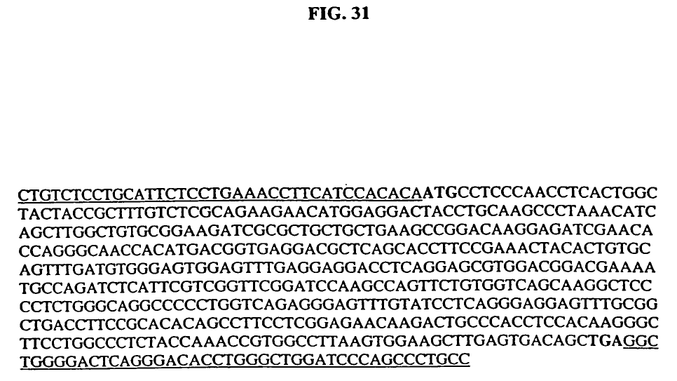

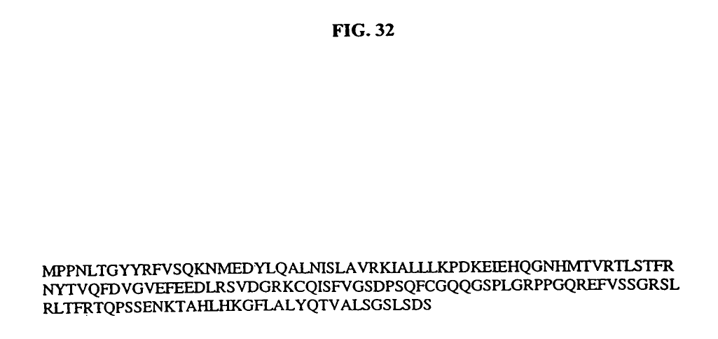

An MEM7 sequence according to the invention includes a nucleic acid sequence encoding a polypeptide related to retinol-binding protein family. The nucleotide sequence [SEQ ID NO:13] of the nucleic acid (Internal Identification No. AC018653_A) encoding a novel protein resembling retinol-binding protein is shown in FIG. 31. An Open Reading Frame (ORF) was identified beginning with an atg initiation codon and ending with a tga termination codon. Putative untranslated regions upstream from the initiation codon and downstream from the termination codon are shown by underlining, and the start and stop codons are shown in bold letters. The amino acid sequence [SEQ ID NO:14] of the encoded protein is presented using the one-letter code in FIG. 32.

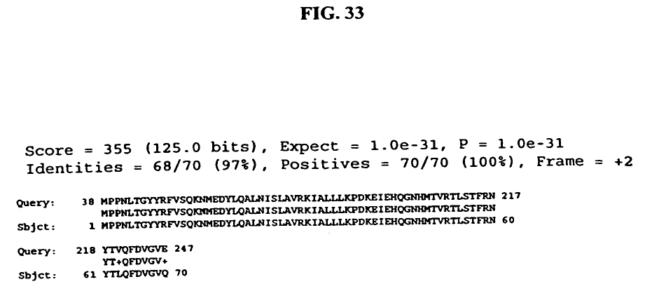

In a search of sequence databases, it was found, e.g., that the MEM7 amino acid sequence [SEQ ID NO:14] of the protein of the invention had 68 of 70 amino acid residues (97%) identical to, and 70 of 70 residues (100%) positive with, the Human cytostatin I protein (patp:W27561; see, FIG. 33).

The novel nucleic acid of the invention encoding a protein resembling retinol-binding protein includes the nucleic acid whose sequence [SEQ ID NO:13] is provided in FIG. 31, or a fragment thereof. The invention also includes a mutant or variant nucleic acid any of whose bases may be changed from the corresponding base shown in FIG. 31, while still encoding a protein that maintains its proteins resembling retinol-binding activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to 20% or more of the bases may be so changed.

The novel protein of the invention includes the proteins resembling retinol-binding protein whose sequence [SEQ ID NO:14] is provided in FIG. 32. The invention also includes a mutant or variant protein any of whose residues may be changed from the corresponding residue shown in FIG. 32, while still encoding a protein that maintains its proteins resembling retinol-binding activities and physiological functions, or a functional fragment thereof. In the mutant or variant protein, up to 20% or more of the residues may be so changed. The invention further encompasses antibodies and antibody fragments, such as Fab or (Fab)₂, that bind immunospecifically to any of the proteins of the invention.

MEM8

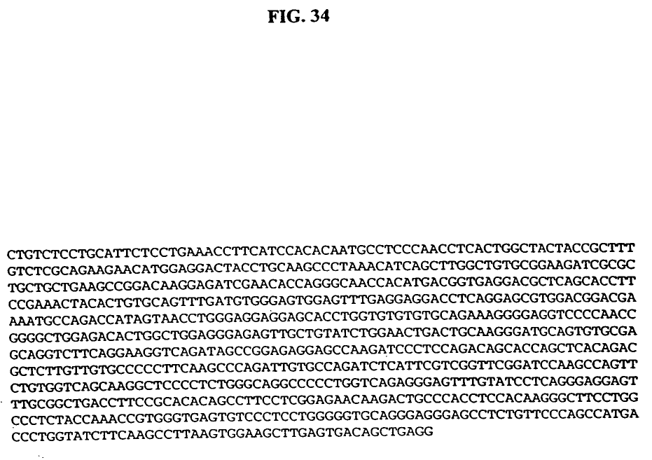

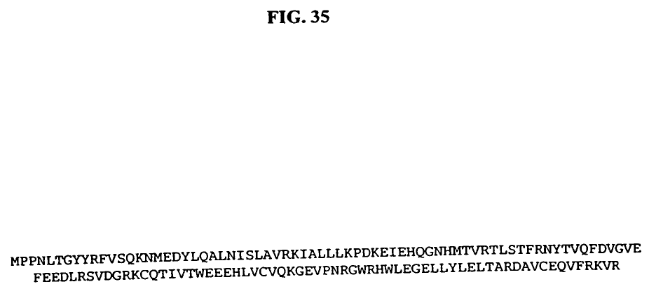

An MEM8 sequence according to the invention includes a nucleic acid sequence encoding a polypeptide related to retinol-binding protein family. The nucleotide sequence [SEQ ID NO:15] of the novel nucleic acid (designated CuraGen Acc. No. AC018653A_da1) encoding a novel protein resembling retinol-binding protein is shown in FIG. 34. An Open Reading Frame (ORF) was identified beginning with an atg initiation codon and ending with a tga termination codon. Putative untranslated regions upstream from the initiation codon and downstream from the termination codon are shown by underlining, and the start and stop codons are shown in bold letters. The amino acid sequence [SEQ ID NO:16] of the encoded protein is presented using the one-letter code in FIG. 35.

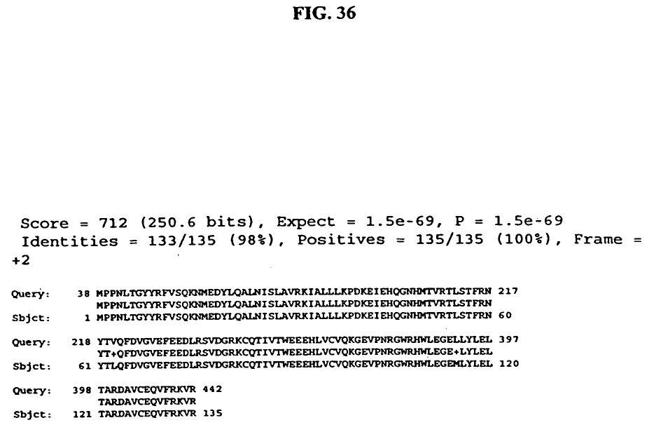

In both a database analysis (see, FIG. 36), the amino acid sequence [SEQ ID NO:16] of the protein of the invention was found to have 135 of 135 amino acid residues (100%) positive with, and 133 of 135 residues (98%) identical to, the 135 amino acid residue Human cytostatin III protein (patp:W30891).

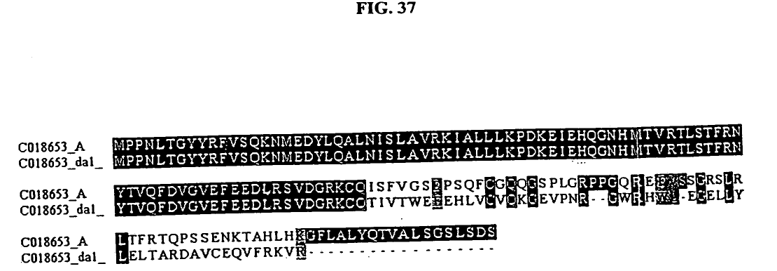

A multiple sequence alignment is illustrated in FIG. 37, with the protein of the invention being shown on Line 2, in a ClustalW analysis comparing the protein of the invention with related protein sequences.

The novel nucleic acid of the invention encoding a protein resembling retinol-binding protein includes the nucleic acid whose sequence [SEQ ID NO:15] is provided in FIG. 34, or a fragment thereof. The invention also includes a mutant or variant nucleic acid any of whose bases may be changed from the corresponding base shown in FIG. 34, while still encoding a protein that maintains its proteins resembling retinol-binding activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to 20% or more of the bases may be so changed.

The novel protein of the invention includes the proteins resembling retinol-binding protein whose sequence [SEQ ID NO:16] is provided in FIG. 35. The invention also includes a mutant or variant protein any of whose residues may be changed from the corresponding residue shown in FIG. 35, while still encoding a protein that maintains its proteins resembling retinol-binding activities and physiological functions, or a functional fragment thereof. In the mutant or variant protein, up to 20% or more of the residues may be so changed. The invention further encompasses antibodies and antibody fragments, such as F_{ab} or (F_{ab})_{2,} that bind immunospecifically to any of the proteins of the invention.

MEMX Nucleic Acids

The nucleic acids of the invention include those that encode a MEMX polypeptide or protein. As used herein, the terms polypeptide and protein are interchangeable.

In some embodiments, a MEMX nucleic acid encodes a mature MEMX polypeptide. As used herein, a “mature” form of a polypeptide or protein described herein relates to the product of a naturally occurring polypeptide or precursor form or proprotein. The naturally occurring polypeptide, precursor or proprotein includes, by way of non-limiting example, the full length gene product, encoded by the corresponding gene. Alternatively, it may be defined as the polypeptide, precursor or proprotein encoded by an open reading frame described herein. The product “mature” form arises, again by way of non-limiting example, as a result of one or more naturally occurring processing steps that may take place within the cell in which the gene product arises. Examples of such processing steps leading to a “mature” form of a polypeptide or protein include the cleavage of the amino-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the amino-terminal methionine, would have residues 2 through N remaining after removal of the amino-terminal methionine. Alternatively, a mature form arising from a precursor polypeptide or protein having residues 1 to N, in which an amino-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

Among the MEMX nucleic acids is the nucleic acid whose sequence is provided in SEQ ID NO:1, 3, 5, 7, 9, 11, 13, or 15, or a fragment thereof. Additionally, the invention includes mutant or variant nucleic acids of SEQ ID NO:1, 3, 5, 7, 9, 11, 13, or 15, or a fragment thereof, any of whose bases may be changed from the corresponding bases shown in SEQ ID NO:1, 3, 5, 7, 9, 11, 13, or 15, while still encoding a protein that maintains at least one of its MEMX-like activities and physiological functions (i.e., modulating angiogenesis, neuronal development). The invention further includes the complement of the nucleic acid sequence of SEQ ID NO:1, 3, 5, 7, 9, 11, 13, or 15, including fragments, derivatives, analogs and homologs thereof. The invention additionally includes nucleic acids or nucleic acid fragments, or complements thereto, whose structures include chemical modifications.

One aspect of the invention pertains to isolated nucleic acid molecules that encode MEMX proteins or biologically active portions thereof. Also included are nucleic acid fragments sufficient for use as hybridization probes to identify MEMX-encoding nucleic acids (e.g., MEMX mRNA) and fragments for use as polymerase chain reaction (PCR) primers for the amplification or mutation of MEMX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

The term “probes” refer to nucleic acid sequences of variable length, preferably between at least about 10 nucleotides (nt), 100 nt, or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated MEMX nucleic acid molecule can contain less than about 50 kb, 25 kb, 5 kb, 4 kb, 3 kb, 2 kb,

1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of SEQ ID NO:1, 3, 5, 7, 9, 11, 13, or 15, or a complement of any of this nucleotide sequence, can be isolated using standard molecular biology techniques and the sequence information provided herein. Using all or a portion of the nucleic acid sequence of SEQ ID NO:1, 3, 5, 7, 9, 11, 13, or 15, as a hybridization probe, MEMX nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to MEMX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of SEQ ID NO:1, 3, 5, 7, 9, 11, 13, or 15, or a complement thereof. Oligonucleotides may be chemically synthesized and may be used as probes.

In another embodiment, an isolated nucleic acid molecule of the invention comprises a nucleic acid molecule that is a complement of the nucleotide sequence shown in SEQ ID NO:1, 3, 5, 7, 9, 11, 13, or 15, or a portion of this nucleotide sequence. A nucleic acid molecule that is complementary to the nucleotide sequence shown in SEQ ID NO:1, 3, 5, 7, 9, 11, 13, or 15, is one that is sufficiently complementary to the nucleotide sequence shown in SEQ ID NO:1, 3, 5, 7, 9, 11, 13, or 15 that it can hydrogen bond with little or no mismatches to the nucleotide sequence shown in SEQ ID NO:1, 3, 5, 7, 9, 11, 13, or 15, thereby forming a stable duplex.

As used herein, the term “complementary” refers to Watson-Crick or Hoogsteen base pairing between nucleotide units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of SEQ ID NO:1, 3, 5, 7, 9, 11, 13, or 15, e.g., a fragment that can be used as a probe or primer, or a fragment encoding a biologically active portion of MEMX. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild-type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, 82%, 90%, 92%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (1981. Adv. Appl. Math. 2: 482-489, which is incorporated herein by reference in its entirety).

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of a MEMX polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a MEMX polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding human MEMX protein. Homologous nucleic acid sequences include those nucleic acid sequences that encode conservative amino acid substitutions (see below) in SEQ ID NO:2, 4, 6, 8, 10, 12, 14, or 16, as well as a polypeptide having MEMX activity. Biological activities of the MEMX proteins are described below. A homologous amino acid sequence does not encode the amino acid sequence of a human MEMX polypeptide.

The nucleotide sequence determined from the cloning of the human MEMX gene allows for the generation of probes and primers designed for use in identifying and/or cloning MEMX homologues in other cell types, e.g., from other tissues, as well as MEMX homologues from other mammals. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 or more consecutive sense strand nucleotide sequence of SEQ ID NO:1, 3, 5, 7, 9, 11, 13, or 15; or an anti-sense strand nucleotide sequence of SEQ ID NO:1, 3, 5, 7, 9, 11, 13, or 15, or of a naturally occurring mutant of SEQ ID NO:1, 3, 5, 7, 9, 11, 13, or 15.

Probes based upon the human MEMX nucleotide sequence can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a MEMX protein, such as by measuring a level of a MEMX-encoding nucleic acid in a sample of cells from a subject e.g., detecting MEMX mRNA levels or determining whether a genomic MEMX gene has been mutated or deleted.

A “polypeptide having a biologically active portion of MEMX” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of MEMX” can be prepared by isolating a portion of SEQ ID NO:1, 3, 5, 7, 9, 11, 13, or 15, that encodes a polypeptide having a MEMX biological activity (biological activities of the MEMX proteins are described below), expressing the encoded portion of MEMX protein (e.g., by recombinant expression in vitro) and assessing the activity of the encoded portion of MEMX. For example, a nucleic acid fragment encoding a biologically active portion of MEMX can optionally include an ATP-binding domain. In another embodiment, a nucleic acid fragment encoding a biologically active portion of MEMX includes one or more regions.

MEMX Variants

The invention further encompasses nucleic acid molecules that differ from the nucleotide sequences shown in SEQ ID NO:1, 3, 5, 7, 9, 11, 13, or 15 due to the degeneracy of the genetic code. These nucleic acids thus encode the same MEMX protein as that encoded by the nucleotide sequence shown in SEQ ID NO:1, 3, 5, 7, 9, 11, 13, or 15, e.g., the polypeptide of SEQ ID NO:2, 4, 6, 8, 10, 12, 14, or 16.

In addition to the human MEMX nucleotide sequence shown in SEQ ID NO:1, 3, 5, 7, 9, 11, 13, or 15, it will be appreciated by those skilled in the art that DNA sequence polymorphisms that lead to changes in the amino acid sequences of MEMX may exist within a population (e.g., the human population). Such genetic polymorphism in the MEMX gene may exist among individuals within a population due to natural allelic variation. As used herein, the terms “gene” and “recombinant gene” refer to nucleic acid molecules comprising an open reading frame encoding a MEMX protein, preferably a mammalian MEMX protein. Such natural allelic variations can typically result in 1-20% variance in the nucleotide sequence of the MEMX gene. Any and all such nucleotide variations and resulting amino acid polymorphisms in MEMX that are the result of natural allelic variation and that do not alter the functional activity of MEMX are intended to be within the scope of the invention.

Moreover, nucleic acid molecules encoding MEMX proteins from other species, and thus that have a nucleotide sequence that differs from the human sequence of SEQ ID NO:1, 3, 5, 7, 9, 11, 13, or 15 are intended to be within the scope of the invention. Nucleic acid molecules corresponding to natural allelic variants and homologues of the MEMX cDNAs of the invention can be isolated based on their homology to the human MEMX nucleic acids disclosed herein using the human cDNAs, or a portion thereof, as a hybridization probe according to standard hybridization techniques under stringent hybridization conditions. For example, a soluble human MEMX cDNA can be isolated based on its homology to human membrane-bound MEMX. Likewise, a membrane-bound human MEMX cDNA can be isolated based on its homology to soluble human MEMX.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:1, 3, 5, 7, 9, 11, 13, or 15. In another embodiment, the nucleic acid is at least 10, 25, 50, 100, 250, 500 or 750 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding MEMX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tₘ) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 620%, 70%, 72%, 82%, 90%, 92%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of SEQ ID NO:1, 3, 5, 7, 9, 11, 13, or 15, corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:1, 3, 5, 7, 9, 11, 13, or 15, or fragments, analogs or derivatives thereof, under conditions of moderate stringency is provided. A non-limiting example of moderate stringency hybridization conditions are hybridization in 6×SSC, 5× Denhardt's solution, 0.20% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of SEQ ID NO:1, 3, 5, 7, 9, 11, 13, or 15, or fragments, analogs or derivatives thereof, under conditions of low stringency, is provided. A non-limiting example of low stringency hybridization conditions are hybridization in 320% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981, Proc Natl Acad Sci USA 78: 6789-6792.

I. Conservative Mutations

In addition to naturally-occurring allelic variants of the MEMX sequence that may exist in the population, the skilled artisan will further appreciate that changes can be introduced by mutation into the nucleotide sequence of SEQ ID NO:1, 3, 5, 7, 9, 11, 13, or 15, thereby leading to changes in the amino acid sequence of the encoded MEMX protein, without altering the functional ability of the MEMX protein. For example, nucleotide substitutions leading to amino acid substitutions at “non-essential” amino acid residues can be made in the sequence of SEQ ID NO:1, 3, 5, 7, 9, 11, 13, or 15. A “non-essential” amino acid residue is a residue that can be altered from the wild-type sequence of MEMX without altering the biological activity, whereas an “essential” amino acid residue is required for biological activity. For example, amino acid residues that are conserved among the MEMX proteins of the present invention, are predicted to be particularly unamenable to alteration.

Another aspect of the invention pertains to nucleic acid molecules encoding MEMX proteins that contain changes in amino acid residues that are not essential for activity. Such MEMX proteins differ in amino acid sequence from SEQ ID NO:2, 4, 6, 8, 10, 12, 14, or 16, yet retain biological activity. In one embodiment, the isolated nucleic acid molecule comprises a nucleotide sequence encoding a protein, wherein the protein comprises an amino acid sequence at least about 720% homologous to the amino acid sequence of SEQ ID NO:2, 4, 6, 8, 10, 12, 14, or 16. Preferably, the protein encoded by the nucleic acid is at least about 80% homologous to SEQ ID NO:2, 4, 6, 8, 10, 12, 14, or 16, more preferably at least about 90%, 92%, 98%, and most preferably at least about 99% homologous to SEQ ID NO:2, 4, 6, 8, 10, 12, 14, or 16.

An isolated nucleic acid molecule encoding a MEMX protein homologous to the protein of can be created by introducing one or more nucleotide substitutions, additions or deletions-into the nucleotide sequence of SEQ ID NO:1, 3, 5, 7, 9, 11, 13, or 5, such that one or more amino acid substitutions, additions or deletions are introduced into the encoded protein.

Mutations can be introduced into the nucleotide sequence of SEQ ID NO:1, 3, 5, 7, 9, 11, 13, or 15 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in MEMX is replaced with another amino acid residue from the same side chain family. Alternatively, in another embodiment, mutations can be introduced randomly along all or part of a MEMX coding sequence, such as by saturation mutagenesis, and the resultant mutants can be screened for MEMX biological activity to identify mutants that retain activity. Following mutagenesis of SEQ ID NO:1, 3, 5, 7, 9, 11, 13, or 15, the encoded protein can be expressed by any recombinant technology known in the art and the activity of the protein can be determined.

In one embodiment, a mutant MEMX protein can be assayed for: (i) the ability to form protein:protein interactions with other MEMX proteins, other cell-surface proteins, or biologically active portions thereof; (ii) complex formation between a mutant MEMX protein and a MEMX receptor; (iii) the ability of a mutant MEMX protein to bind to an intracellular target protein or biologically active portion thereof; (e.g., avidin proteins); (iv) the ability to bind MEMX protein; or (v) the ability to specifically bind an anti-MEMX protein antibody.

Antisense MEMX Nucleic Acids

Another aspect of the invention pertains to isolated antisense nucleic acid molecules that are hybridizable to or complementary to the nucleic acid molecule comprising the nucleotide sequence of SEQ ID NO:1, 3, 5, 7, 9, 11, 13, or 15, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire MEMX coding strand, or to only a portion thereof. Nucleic acid molecules encoding fragments, homologs, derivatives and analogs of a MEMX protein of SEQ ID NO:2, 4, 6, 8, 10, 12, 14, or 16, or antisense nucleic acids complementary to a MEMX nucleic acid sequence of SEQ ID NO:1, 3, 5, 7, 9, 11, 13, or 15 are additionally provided.

In one embodiment, an antisense nucleic acid molecule is antisense to a “coding region” of the coding strand of a nucleotide sequence encoding MEMX. The term “coding region” refers to the region of the nucleotide sequence comprising codons which are translated into amino acid residues (e.g., the protein coding region of human MEMX corresponds to SEQ ID NO:2, 4, 6, 8, 10, 12, 14, or 16). In another embodiment, the antisense nucleic acid molecule is antisense to a “non-coding region” of the coding strand of a nucleotide sequence encoding MEMX. The term “non-coding region” refers to 5′ and 3′ sequences which flank the coding region that are not translated into amino acids (i.e., also referred to as 5′ and 3′ untranslated regions).

Given the coding strand sequences encoding MEMX disclosed herein (e.g., SEQ ID NO:1, 3, 5, 7, 9, 11, 13, or 15), antisense nucleic acids of the invention can be designed according to the rules of Watson and Crick or Hoogsteen base pairing. The antisense nucleic acid molecule can be complementary to the entire coding region of MEMX mRNA, but more preferably is an oligonucleotide that is antisense to only a portion of the coding or non-coding region of MEMX mRNA. For example, the antisense oligonucleotide can be complementary to the region surrounding the translation start site of MEMX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a MEMX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (see, Gaultier, et al. 1987. Nucl. Acids Res. 15: 6625-6641). The antisense nucleic acid molecule can also comprise a 2′-o-methylribonucleotide (see, Inoue, et al., 1987. Nucl. Acids Res. 15: 6131-6148) or a chimeric RNA-DNA analogue (see, Inoue, et al., 1987. FEBS Lett. 215: 327-330).

Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

MEMX Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as a mRNA, to which they have a complementary region. Thus, ribozymes (e.g., hammerhead ribozymes; see, Haselhoff and Gerlach, 1988. Nature 334: 585-591) can be used to catalytically-cleave MEMX mRNA transcripts to thereby inhibit translation of MEMX mRNA. A ribozyme having specificity for a MEMX-encoding nucleic acid can be designed based upon the nucleotide sequence of a MEMX DNA disclosed herein (i.e., SEQ ID NO:1, 3, 5, 7, 9, 11, 13, or 15). For example, a derivative of a Tetrahymena L-19 IVS RNA can be constructed in which the nucleotide sequence of the active site is complementary to the nucleotide sequence to be cleaved in a MEMX-encoding mRNA. See, e.g., Cech, et al. U.S. Pat. No. 4,987,071; and Cech, et al., U.S. Pat. No. 5,116,742. Alternatively, MEMX mRNA can be used to select a catalytic RNA having a specific ribonuclease activity from a pool of RNA molecules. See, e.g., Bartel, et al., 1993. Science 261: 1411-1418.

Alternatively, MEMX gene expression can be inhibited by targeting nucleotide sequences complementary to the regulatory region of the MEMX (e.g., the MEMX promoter and/or enhancers) to form triple helical structures that prevent transcription of the MEMX gene in target cells. See, generally, Helene, 1991. Anticancer Drug Des. 6: 569-584; Helene. et al. 1992. Ann. N.Y. Acad. Sci. 660: 27-36; and Maher. 1992. Bioassays 14: 807-815.

In various embodiments, the nucleic acids of MEMX can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see, Hyrup, et al. 1996. Bioorg. Med. Chem. 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of MEMX can be used in therapeutic and diagnostic applications. For example, PNAs can be used as antisense or antigene agents for sequence-specific modulation of gene expression by, e.g., inducing transcription or translation arrest or inhibiting replication. PNAs of MEMX can also be used, e.g., in the analysis of single base pair mutations in a gene by, e.g., PNA directed PCR clamping; as artificial restriction enzymes when used in combination with other enzymes, e.g., S1 nucleases (Hyrup, 1996. supra); or as probes or primers for DNA sequence and hybridization (Hyrup, 1996 and Perry-O'Keefe, 1996., supra).

In another embodiment, PNAs of MEMX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of MEMX can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation. The synthesis of PNA-DNA chimeras can be performed (see, e.g., Finn, et al., 1996. Nucl. Acids Res. 24: 3357-3363. For example, a DNA chain can be synthesized on a solid support using standard phosphoramidite coupling chemistry, and modified nucleoside analogs, e.g., 5′-(4-methoxytrityl) amino-5′-deoxy-thymidine phosphoramidite, can be used between the PNA and the 5′ end of DNA (see, Mag, et al., 1989. Nucl. Acids Res. 17: 5973-5988). PNA monomers are then coupled in a stepwise manner to produce a chimeric molecule with a 5′ PNA segment and a 3′ DNA segment (Finn, et al., 1996., supra). Alternatively, chimeric molecules can be synthesized with a 5′ DNA segment and a 3′ PNA segment (see, Petersen, et al., 1975. Bioorg. Med. Chem. Lett. 5: 1119-1124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

MEMX Polypeptides

A MEMX polypeptide of the invention includes the MEMX-like protein whose sequence is provided in SEQ ID NO:2, 4, 6, 8, 10, 12, 14, or 16. The invention also includes a mutant or variant protein any of whose residues may be changed from the corresponding residue shown in SEQ ID NO:2, 4, 6, 8, 10, 12, 14, or 16, while still encoding a protein that maintains its MEMX-like activities and physiological functions, or a functional fragment thereof. In some embodiments, up to 20% or more of the residues may be so changed in the mutant or variant protein. In some embodiments, the MEMX polypeptide according to the invention is a mature polypeptide.

In general, a MEMX-like variant that preserves MEMX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated MEMX proteins, and biologically active portions thereof, or derivatives, fragments, analogs or homologs thereof. Also provided are polypeptide fragments suitable for use as immunogens to raise anti-MEMX antibodies. In one embodiment, native MEMX proteins can be isolated from cells or tissue sources by an appropriate purification scheme using standard protein purification techniques. In another embodiment, MEMX proteins are produced by recombinant DNA techniques. Alternative to recombinant expression, a MEMX protein or polypeptide can be synthesized chemically using standard peptide synthesis techniques.

An “isolated” or “purified” protein or biologically active portion thereof is substantially free of cellular material or other contaminating proteins from the cell or tissue source from which the MEMX protein is derived, or substantially free from chemical precursors or other chemicals when chemically synthesized. The language “substantially free of cellular material” includes preparations of MEMX protein in which the protein is separated from cellular components of the cells from which it is isolated or recombinantly produced. In one embodiment, the language “substantially free of cellular material” includes preparations of MEMX protein having less than about 30% (by dry weight) of non-MEMX protein (also referred to herein as a “contaminating protein”), more preferably less than about 20% of non-MEMX protein, still more preferably less than about 10% of non-MEMX protein, and most preferably less than about 20% non-MEMX protein. When the MEMX protein or biologically active portion thereof is recombinantly produced, it is also preferably substantially free of culture medium, i.e., culture medium represents less than about 20%, more preferably less than about 10%, and most preferably less than about 20% of the volume of the protein preparation.

The language “substantially free of chemical precursors or other chemicals” includes preparations of MEMX protein in which the protein is separated from chemical precursors or other chemicals that are involved in the synthesis of the protein. In one embodiment, the language “substantially free of chemical precursors or other chemicals” includes preparations of MEMX protein having less than about 30% (by dry weight) of chemical precursors or non-MEMX chemicals, more preferably less than about 20% chemical precursors or non-MEMX chemicals, still more preferably less than about 10% chemical precursors or non-MEMX chemicals, and most preferably less than about 20% chemical precursors or non-MEMX chemicals.

Biologically active portions of a MEMX protein include peptides comprising amino acid sequences sufficiently homologous to or derived from the amino acid sequence of the MEMX protein, e.g., the amino acid sequence shown in SEQ ID NO:2, 4, 6, 8, 10, 12, 14, or 16, that include fewer amino acids than the full length MEMX proteins, and exhibit at least one activity of a MEMX protein. Typically, biologically active portions comprise a domain or motif with at least one activity of the MEMX protein. A biologically active portion of a MEMX protein can be a polypeptide which is, for example, 10, 25, 50, 100 or more amino acids in length.

A biologically active portion of a MEMX protein of the present invention may contain at least one of the above-identified domains conserved between the MEMX proteins, e.g. TSR modules. Moreover, other biologically active portions, in which other regions of the protein are deleted, can be prepared by recombinant techniques and evaluated for one or more of the functional activities of a native MEMX protein.

In an embodiment, the MEMX protein has an amino acid sequence shown in SEQ ID NO:2, 4, 6, 8, 10, 12, 14, or 16. In other embodiments, the MEMX protein is substantially homologous to SEQ ID NO:2, 4, 6, 8, 10, 12, 14, or 16 and retains the functional activity of the protein of SEQ ID NO:2, 4, 6, 8, 10, 12, 14, or 16, yet differs in amino acid sequence due to natural allelic variation or mutagenesis, as described in detail below. Accordingly, in another embodiment, the MEMX protein is a protein that comprises an amino acid sequence at least about 45% homologous, and more preferably about 55, 65, 70, 75, 80, 85, 90, 95, 98, or even 99% homologous to the amino acid sequence of SEQ ID NO:2, 4, 6, 8, 10, 12, 14, or 16 and retains the functional activity of the MEMX proteins of SEQ ID NO:2, 4, 6, 8, 10, 12, 14, or 16.

I. Determining Homology Between Two or More Amino Acid Sequences

To determine the percent homology of two amino acid sequences or of two nucleic acids, the sequences are aligned for optimal comparison purposes (e.g., gaps can be introduced in either of the sequences being compared for optimal alignment between the sequences). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 72%, 80%, 82%, 90%, 92%, 98%, or 99%, with the CDS (encoding) part of the DNA sequence shown in SEQ ID NO:1, 3, 5, 7, 9, 11, 13, or 15.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. The term “percentage of positive residues” is calculated by comparing two optimally aligned sequences over that region of comparison, determining the number of positions at which the identical and conservative amino acid substitutions, as defined above, occur in both sequences to yield the number of matched positions, dividing the number of matched positions by the total number of positions in the region of comparison (i.e., the window size), and multiplying the result by 100 to yield the percentage of positive residues.

Chimeric and Fusion Proteins

The invention also provides MEMX chimeric or fusion proteins. As used herein, a MEMX “chimeric protein” or “fusion protein” comprises a MEMX polypeptide operatively linked to a non-MEMX polypeptide. An “MEMX polypeptide” refers to a polypeptide having an amino acid sequence corresponding to MEMX, whereas a “non-MEMX polypeptide” refers to a polypeptide having an amino acid sequence corresponding to a protein that is not substantially homologous to the MEMX protein, e.g., a protein that is different from the MEMX protein and that is derived from the same or a different organism. Within a MEMX fusion protein the MEMX polypeptide can correspond to all or a portion of a MEMX protein. In one embodiment, a MEMX fusion protein comprises at least one biologically active portion of a MEMX protein. In another embodiment, a MEMX fusion protein comprises at least two biologically active portions of a MEMX protein. Within the fusion protein, the term “operatively linked” is intended to indicate that the MEMX polypeptide and the non-MEMX polypeptide are fused in-frame to each other. The non-MEMX polypeptide can be fused to the N-terminus or C-terminus of the MEMX polypeptide.

For example, in one embodiment a MEMX fusion protein comprises a MEMX polypeptide operably linked to the extracellular domain of a second protein. Such fusion proteins can be further utilized in screening assays for compounds that modulate MEMX activity (such assays are described in detail below).

In another embodiment, the fusion protein is a glutathione S-transferase (GST)-MEMX fusion protein in which the MEMX sequences are fused to the carboxyl-terminus of the GST sequences. Such fusion proteins can facilitate the purification of recombinant MEMX.

In another embodiment, the fusion protein is a MEMX-immunoglobulin fusion protein in which the MEMX sequences comprising one or more domains are fused to sequences derived from a member of the immunoglobulin protein family. The MEMX-immunoglobulin fusion proteins of the invention can be incorporated into pharmaceutical compositions and administered to a subject to inhibit an interaction between a MEMX ligand and a MEMX protein on the surface of a cell, to thereby suppress MEMX-mediated signal transduction in vivo. In one non-limiting example, a contemplated MEMX ligand of the invention is the MEMX receptor. The MEMX-immunoglobulin fusion proteins can be used to affect the bioavailability of a MEMX cognate ligand. Inhibition of the MEMX ligand/MEMX interaction may be useful therapeutically for both the treatment of proliferative and differentiative disorders, e,g., cancer as well as modulating (e.g., promoting or inhibiting) cell survival. Moreover, the MEMX-immunoglobulin fusion proteins of the invention can be used as immunogens to produce anti-MEMX antibodies in a subject, to purify MEMX ligands, and in screening assays to identify molecules that inhibit the interaction of MEMX with a MEMX ligand.

A MEMX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A MEMX-encoding nucleic acid can be cloned into such an expression vector such that the fusion moiety is linked in-frame to the MEMX protein.

MEMX Agonists and Antagonists

The present invention also pertains to variants of the MEMX proteins that function as either MEMX agonists (i.e., mimetics) or as MEMX antagonists. Variants of the MEMX protein can be generated by mutagenesis, e.g., discrete point mutation or truncation of the MEMX protein. An agonist of the MEMX protein can retain substantially the same, or a subset of, the biological activities of the naturally occurring form of the MEMX protein. An antagonist of the MEMX protein can inhibit one or more of the activities of the naturally occurring form of the MEMX protein by, for example, competitively binding to a downstream or upstream member of a cellular signaling cascade which includes the MEMX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the MEMX proteins.

Variants of the MEMX protein that function as either MEMX agonists (mimetics) or as MEMX antagonists can be identified by screening combinatorial libraries of mutants, e.g., truncation mutants, of the MEMX protein for MEMX protein agonist or antagonist activity. In one embodiment, a variegated library of MEMX variants is generated by combinatorial mutagenesis at the nucleic acid level and is encoded by a variegated gene library. A variegated library of MEMX variants can be produced by, for example, enzymatically ligating a mixture of synthetic oligonucleotides into gene sequences such that a degenerate set of potential MEMX sequences is expressible as individual polypeptides, or alternatively, as a set of larger fusion proteins (e.g., for phage display) containing the set of MEMX sequences therein. There are a variety of methods which can be used to produce libraries of potential MEMX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential MEMX sequences. Methods for synthesizing degenerate oligonucleotides are known in the art (see, e.g., Narang 1983. Tetrahedron 39:3; Itakura, et al., 1984. Annual Rev. Biochem. 53: 323; Itakura, et al., 1984. Science 198: 1056; Ike, et al., 1983. Nucl. Acid Res. 11:477.

I. Polypeptide Libraries

In addition, libraries of fragments of the MEMX protein coding sequences can be used to generate a variegated population of MEMX fragments for screening and subsequent selection of variants of an MEMX protein. In one embodiment, a library of coding sequence fragments can be generated by treating a double stranded PCR fragment of an MEMX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S1 nuclease, and ligating the resulting fragment library into an expression vector. By this method, expression libraries can be derived which encodes N-terminal and internal fragments of various sizes of the MEMX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of MEMX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify MEMX variants. See, e.g., Arkin and Yourvan, 1992. Proc. Natl. Acad. Sci. USA 89: 7811-7815; Delgrave, et al., 1993. Protein Engineering 6:327-331.

Anti-MEMX Antibodies

Also included in the invention are antibodies to MEMX proteins, or fragments of MEMX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated MEMX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence shown in SEQ ID NO:2, 4, 6, 8, 10, 12, 14, or 16, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of MEMX-related protein that is located on the surface of the protein, e.g., a hydrophilic region. A hydrophobicity analysis of the human MEMX-related protein sequence will indicate which regions of a MEMX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle, 1982. J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, ANTIBODIES: A LABORATORY MANUAL, Harlow E, and Lane D, 1988, Cold Spring Harbor Laboratory Press, Cold Spring Harbor, N.Y., incorporated herein by reference). Some of these antibodies are discussed below.

I. Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

II. Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, (1975. Nature 256: 495). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, 1984. J. Immunol. 133: 3001; Brodeur, et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, (1980. Anal. Biochem. 107: 220). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, 1994. Nature 368: 812-813) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

III. Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones, et al., 1986. Nature, 321: 522-525; Riechmann, et al., 1988. Nature 332: 323-327; Verhoeyen, et al., 1988. Science, 239: 1534-1536); by substituting rodent CDRs or CDR sequences for the corresponding sequences of a human antibody. (see, e.g., U.S. Pat. No. 5,225,539.). In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones, et al., 1986, supra; Riechmann, et al., 1988, supra; Presta, 1992. Curr. Op. Struct. Biol., 2: 593-596).

IV. Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see, e.g., Kozbor, et al., 1983. Immunol Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see, e.g., Cote, et al., 1983. Proc. Natl. Acad. Sci. USA 80: 2026-2030) or by transforming human B-cells with Epstein Barr Virus in vitro (see, e.g., Cole, et al., 1985., supra).

In addition, human antibodies can also be produced using additional techniques, including phage display libraries (Hoogenboom and Winter, 1991. J. Mol. Biol. 227: 381; Marks, et al., J. Mol. Biol. 222:). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and Marks, et al. (1992. Bio/Technology 10: 779-783); Lonberg, et al. (1994. Nature 368: 856-859); Morrison (1994. Nature 368: 812-813); Fishwild, et al, (1996. Nature Biotech. 14: 845-851); Neuberger (1996. Nature Biotech. 14: 826); and Lonberg and Huszar (1995. International Rev. Immunol. 13: 65-93).

Human antibodies may additionally be produced using transgenic nonhuman animals which are modified so as to produce fully human antibodies rather than the animal's endogenous antibodies in response to challenge by an antigen. See, PCT Publication WO94/02602. The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT Publications WO 96/33735 and WO 96/34096. This animal produces

B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

V. F_{ab} Fragments and Single Chain Antibodies

According to the invention, techniques can be adapted for the production of single-chain antibodies specific to an antigenic protein of the invention (see, e.g., U.S. Pat. No. 4,946,778). In addition, methods can be adapted for the construction of F_{ab} expression libraries (see, e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent; and (iv) Fᵥ fragments.

VI. Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (see, e.g., Milstein and Cuello, 1983. Nature 305: 537-539). Because of the random assortment of immunoglobulin heavy and light chains, these hybridomas (i.e., quadromas) produce a potential mixture of ten different antibody molecules, of which only one has the correct bispecific structure. The purification of the correct molecule is usually accomplished by affinity chromatography steps. Similar procedures are disclosed in PCT Publication

WO 93/08829 (published May 13, 1993); Traunecker, et al., (1991. EMBO J., 10: 3655-3659).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies (see, e.g., Suresh, et al., 1986. Meth. Enzymology 121: 210).

According to another approach described in PCT Publication WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F_{(ab′)2} bispecific antibodies). Techniques for generating bispecific antibodies from antibody fragments have been described in the literature. For example, bispecific antibodies can be prepared using chemical linkage. Brennan, et al. (1985. Science 229: 81) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby, et al. (1992. J. Exp. Med. 175: 217-225) describe the production of a fully humanized bispecific antibody F_{(ab′)2} molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers (Kostelny, et al., 1992. J. Immunol. 148(5): 1547-1553). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger, et al. (1993. Proc. Natl. Acad. Sci. USA 90: 6444-6448) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported (Gruber, et al., 1994. J. Immunol. 152: 5368).

Antibodies with more than two valencies are contemplated. For example, trispecific antibodies can be prepared (Tutt, et al., 1991. J. Immunol. 147: 60).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g., CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

VII. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving cross-linking agents. For example, immunotoxins can be constructed using a disulfide exchange reaction or by forming a thioether bond. Examples of suitable reagents for this purpose include iminothiolate and methyl-4-mercaptobutyrimidate and those disclosed, e.g., in U.S. Pat. No. 4,676,980.

VIII. Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See, e.g., Caron, et al., 1992. J. Exp Med., 176: 1191-1195; Shopes, 1992. J. Immunol. 148: 2918-2922. Homodimeric antibodies with enhanced anti-tumor activity can also be prepared using heterobifunctional cross-linkers as described by Wolff, et al. (1993. Cancer Res. 53: 2560-2565). Alternatively, an antibody can be engineered that has dual Fc regions and can thereby have enhanced complement lysis and ADCC capabilities (see, e.g., Stevenson, et al., 1989. Anti-Cancer Drug Design 3: 219-230).

IX. Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described by Vitetta, et al. (1987. Science 238: 1098). Carbon-14-labeled, 1-isothiocyanatobenzyl-3-methyldiethylene triaminepentaacetic acid (MX-DTPA) is an exemplary chelating agent for conjugation of radionucleotide to the antibody. See, PCT Publication WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

MEMX Recombinant Expression Vectors and Host Cells

Another aspect of the invention pertains to vectors, preferably expression vectors, containing a nucleic acid encoding an MEMX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., MEMX proteins, mutant forms of MEMX proteins, fusion proteins, etc.).

The recombinant expression vectors of the invention can be designed for expression of MEMX proteins in prokaryotic or eukaryotic cells. For example, MEMX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the MEMX expression vector is a yeast expression vector. Examples of vectors for expression in yeast Saccharomyces cerivisae include pYepSec 1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, MEMX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to MEMX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, MEMX protein can be expressed in bacterial cells such as E. coli, insect cells, yeast or mammalian cells (e.g., Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding MEMX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) MEMX protein. Accordingly, the invention further provides methods for producing MEMX protein using the host cells of the invention. In one embodiment, the method comprises culturing the host cell of invention (into which a recombinant expression vector encoding MEMX protein has been introduced) in a suitable medium such that MEMX protein is produced. In another embodiment, the method further comprises isolating MEMX protein from the medium or the host cell.

Transgenic MEMX Animals

The host cells of the invention can also be used to produce non-human transgenic animals. For example, in one embodiment, a host cell of the invention is a fertilized oocyte or an embryonic stem cell into which MEMX protein-coding sequences have been introduced. Such host cells can then be used to create non-human transgenic animals in which exogenous MEMX sequences have been introduced into their genome or homologous recombinant animals in which endogenous MEMX sequences have been altered. Such animals are useful for studying the function and/or activity of MEMX protein and for identifying and/or evaluating modulators of MEMX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous MEMX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing MEMX-encoding nucleic acid into the male pronuclei of a fertilized oocyte (e.g., by microinjection, retroviral infection) and allowing the oocyte to develop in a pseudopregnant female foster animal. The human MEMX cDNA sequences of SEQ ID NO:1, 3, 5, 7, 9, 11, 13, or 15, can be introduced as a transgene into the genome of a non-human animal. Alternatively, a non-human homologue of the human MEMX gene, such as a mouse MEMX gene, can be isolated based on hybridization to the human MEMX cDNA (described further supra) and used as a transgene. Intronic sequences and polyadenylation signals can also be included in the transgene to increase the efficiency of expression of the transgene. A tissue-specific regulatory sequence(s) can be operably-linked to the MEMX transgene to direct expression of MEMX protein to particular cells. Methods for generating transgenic animals via embryo manipulation and microinjection, particularly animals such as mice, have become conventional in the art and are described, for example, in U.S. Pat. No. 4,736,866; No. 4,870,009; and No. 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the MEMX transgene in its genome and/or expression of MEMX mRNA in tissues or cells of the animals. A transgenic founder animal can then be used to breed additional animals carrying the transgene. Moreover, transgenic animals carrying a transgene-encoding MEMX protein can further be bred to other transgenic animals carrying other transgenes.

To create a homologous recombinant animal, a vector is prepared which contains at least a portion of an MEMX gene into which a deletion, addition or substitution has been introduced to thereby alter, e.g., functionally disrupt, the MEMX gene. The MEMX gene can be a human gene (e.g., the cDNA of SEQ ID NO:1, 3, 5, 7, 9, 11, 13, or 15), but more preferably, is a non-human homologue of a human MEMX gene. For example, a mouse homologue of human MEMX gene of SEQ ID NO:1, 3, 5, 7, 9, 11, 13, or 15, can be used to construct a homologous recombination vector suitable for altering an endogenous MEMX gene in the mouse genome. In one embodiment, the vector is designed such that, upon homologous recombination, the endogenous MEMX gene is functionally disrupted (i.e., no longer encodes a functional protein; also referred to as a “knock out” vector).

Alternatively, the vector can be designed such that, upon homologous recombination, the endogenous MEMX gene is mutated or otherwise altered but still encodes functional protein (e.g., the upstream regulatory region can be altered to thereby alter the expression of the endogenous MEMX protein). In the homologous recombination vector, the altered portion of the MEMX gene is flanked at its 5′- and 3′-termini by additional nucleic acid of the MEMX gene to allow for homologous recombination to occur between the exogenous MEMX gene carried by the vector and an endogenous MEMX gene in an embryonic stem cell. The additional flanking MEMX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced MEMX gene has homologously-recombined with the endogenous MEMX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/oxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The MEMX nucleic acid molecules, MEMX proteins, and anti-MEMX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an MEMX protein or anti-MEMX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express MEMX protein (e.g., via a recombinant expression vector in a host cell in gene therapy applications), to detect MEMX mRNA (e.g., in a biological sample) or a genetic lesion in an MEMX gene, and to modulate MEMX activity, as described further, below. In addition, the MEMX proteins can be used to screen drugs or compounds that modulate the MEMX protein activity or expression as well as to treat disorders characterized by insufficient or excessive production of MEMX protein or production of MEMX protein forms that have decreased or aberrant activity compared to MEMX wild-type protein. In addition, the anti-MEMX antibodies of the invention can be used to detect and isolate MEMX proteins and modulate MEMX activity.

The invention further pertains to novel agents identified by the screening assays described herein and uses thereof for treatments as described, supra.

I. Screening Assays

The invention provides a method (also referred to herein as a “screening assay”) for identifying modulators, i.e., candidate or test compounds or agents (e.g., peptides, peptidomimetics, small molecules or other drugs) that bind to MEMX proteins or have a stimulatory or inhibitory effect on, e.g., MEMX protein expression or MEMX protein activity. The invention also includes compounds identified in the screening assays described herein.

In one embodiment, the invention provides assays for screening candidate or test compounds which bind to or modulate the activity of the membrane-bound form of an MEMX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 Kdal and most preferably less than about 4 Kdal. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of MEMX protein, or a biologically-active portion thereof, on the cell surface is contacted with a test compound and the ability of the test compound to bind to an MEMX protein determined. The cell, for example, can of mammalian origin or a yeast cell. Determining the ability of the test compound to bind to the MEMX protein can be accomplished, for example, by coupling the test compound with a radioisotope or enzymatic label such that binding of the test compound to the MEMX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of MEMX protein, or a biologically-active portion thereof, on the cell surface with a known compound which binds MEMX to form an assay mixture, contacting the assay mixture with a test compound, and determining the ability of the test compound to interact with an MEMX protein, wherein determining the ability of the test compound to interact with an MEMX protein comprises determining the ability of the test compound to preferentially bind to MEMX protein or a biologically-active portion thereof as compared to the known compound.

In another embodiment, an assay is a cell-based assay comprising contacting a cell expressing a membrane-bound form of MEMX protein, or a biologically-active portion-thereof, on the cell surface with a test compound and determining the ability of the test compound to modulate (e.g., stimulate or inhibit) the activity of the MEMX protein or biologically-active portion thereof. Determining the ability of the test compound to modulate the activity of MEMX or a biologically-active portion thereof can be accomplished, for example, by determining the ability of the MEMX protein to bind to or interact with an MEMX target molecule. As used herein, a “target molecule” is a molecule with which an MEMX protein binds or interacts in nature, for example, a molecule on the surface of a cell which expresses an MEMX interacting protein, a molecule on the surface of a second cell, a molecule in the extracellular milieu, a molecule associated with the internal surface of a cell membrane or a cytoplasmic molecule. An MEMX target molecule can be a non-MEMX molecule or an MEMX protein or polypeptide of the invention. In one embodiment, an MEMX target molecule is a component of a signal transduction pathway that facilitates transduction of an extracellular signal (e.g. a signal generated by binding of a compound to a membrane-bound MEMX molecule) through the cell membrane and into the cell. The target, for example, can be a second intercellular protein that has catalytic activity or a protein that facilitates the association of downstream signaling molecules with MEMX.

Determining the ability of the MEMX protein to bind to or interact with an MEMX target molecule can be accomplished by one of the methods described above for determining direct binding. In one embodiment, determining the ability of the MEMX protein to bind to or interact with an MEMX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an MEMX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an MEMX protein or biologically-active portion thereof with a test compound and determining the ability of the test compound to bind to the MEMX protein or biologically-active portion thereof. Binding of the test compound to the MEMX protein can be determined either directly or indirectly as described above. In one such embodiment, the assay comprises contacting the MEMX protein or biologically-active portion thereof with a known compound which binds MEMX to form an assay mixture, contacting the assay mixture with a test compound, and determining the ability of the test compound to interact with an MEMX protein, wherein determining the ability of the test compound to interact with an MEMX protein comprises determining the ability of the test compound to preferentially bind to MEMX or biologically-active portion thereof as compared to the known compound.

In still another embodiment, an assay is a cell-free assay comprising contacting MEMX protein or biologically-active portion thereof with a test compound and determining the ability of the test compound to modulate (e.g. stimulate or inhibit) the activity of the MEMX protein or biologically-active portion thereof. Determining the ability of the test compound to modulate the activity of MEMX can be accomplished, for example, by determining the ability of the MEMX protein to bind to an MEMX target molecule by one of the methods described above for determining direct binding. In an alternative embodiment, determining the ability of the test compound to modulate the activity of MEMX protein can be accomplished by determining the ability of the MEMX protein further modulate an MEMX target molecule. For example, the catalytic/enzymatic activity of the target molecule on an appropriate substrate can be determined as described, supra.

In yet another embodiment, the cell-free assay comprises contacting the MEMX protein or biologically-active portion thereof with a known compound which binds MEMX protein to form an assay mixture, contacting the assay mixture with a test compound, and determining the ability of the test compound to interact with an MEMX protein, wherein determining the ability of the test compound to interact with an MEMX protein comprises determining the ability of the MEMX protein to preferentially bind to or modulate the activity of an MEMX target molecule.

The cell-free assays of the invention are amenable to use of both the soluble form or the membrane-bound form of MEMX protein. In the case of cell-free assays comprising the membrane-bound form of MEMX protein, it may be desirable to utilize a solubilizing agent such that the membrane-bound form of MEMX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl—N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either MEMX protein or its target molecule to facilitate separation of complexed from uncomplexed forms of one or both of the proteins, as well as to accommodate automation of the assay. Binding of a test compound to MEMX protein, or interaction of MEMX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-MEMX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or MEMX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of MEMX protein binding or activity determined using standard techniques.

Other techniques for immobilizing proteins on matrices can also be used in the screening assays of the invention. For example, either the MEMX protein or its target molecule can be immobilized utilizing conjugation of biotin and streptavidin. Biotinylated MEMX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with MEMX protein or target molecules, but which do not interfere with binding of the MEMX protein to its target molecule, can be derivatized to the wells of the plate, and unbound target or MEMX protein trapped in the wells by antibody conjugation. Methods for detecting such complexes, in addition to those described above for the GST-immobilized complexes, include immunodetection of complexes using antibodies reactive with the MEMX protein or target molecule, as well as enzyme-linked assays that rely on detecting an enzymatic activity associated with the MEMX protein or target molecule.

In another embodiment, modulators of MEMX protein expression are identified in a method wherein a cell is contacted with a candidate compound and the expression of MEMX mRNA or protein in the cell is determined. The level of expression of MEMX mRNA or protein in the presence of the candidate compound is compared to the level of expression of MEMX mRNA or protein in the absence of the candidate compound. The candidate compound can then be identified as a modulator of MEMX mRNA or protein expression based upon this comparison. For example, when expression of MEMX mRNA or protein is greater (i.e., statistically significantly greater) in the presence of the candidate compound than in its absence, the candidate compound is identified as a stimulator of MEMX mRNA or protein expression. Alternatively, when expression of MEMX mRNA or protein is less (statistically significantly less) in the presence of the candidate compound than in its absence, the candidate compound is identified as an inhibitor of MEMX mRNA or protein expression. The level of MEMX mRNA or protein expression in the cells can be determined by methods described herein for detecting MEMX mRNA or protein.

In yet another aspect of the invention, the MEMX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with MEMX (“MEMX-binding proteins” or “MEMX-bp”) and modulate MEMX activity. Such MEMX-binding proteins are also likely to be involved in the propagation of signals by the MEMX proteins as, for example, upstream or downstream elements of the MEMX pathway.

The two-hybrid system is based on the modular nature of most transcription factors, which consist of separable DNA-binding and activation domains. Briefly, the assay utilizes two different DNA constructs. In one construct, the gene that codes for MEMX is fused to a gene encoding the DNA binding domain of a known transcription factor (e.g., GALA). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an MEMX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with MEMX.

The invention further pertains to novel agents identified by the aforementioned screening assays and uses thereof for treatments as described herein.

II. Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the MEMX sequences, SEQ ID NO:1, 3, 5, 7, 9, 11, 13, or 15, or fragments or derivatives thereof, can be used to map the location of the MEMX genes, respectively, on a chromosome. The mapping of the MEMX sequences to chromosomes is an important first step in correlating these sequences with genes associated with disease.

Briefly, MEMX genes can be mapped to chromosomes by preparing PCR primers (preferably 15-25 bp in length) from the MEMX sequences. Computer analysis of the MEMX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the MEMX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the MEMX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to non-coding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the MEMX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The MEMX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for Restriction Fragment Length Polymorphisms (RFLP) described in U.S. Pat. No. 5,272,057.

Furthermore, the sequences of the invention can be used to provide an alternative technique that determines the actual base-by-base DNA sequence of selected portions of an individual's genome. Thus, the MEMX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The MEMX sequences of the invention uniquely represent portions of the human genome. Allelic variation occurs to some degree in the coding regions of these sequences, and to a greater degree in the non-coding regions. It is estimated that allelic variation between individual humans occurs with a frequency of about once per each 500 bases. Much of the allelic variation is due to single nucleotide polymorphisms (SNPs), which include RFLPs.

Each of the sequences described herein can, to some degree, be used as a standard against which DNA from an individual can be compared for identification purposes. Because greater numbers of polymorphisms occur in the non-coding regions, fewer sequences are necessary to differentiate individuals. The non-coding sequences can comfortably provide positive individual identification with a panel of perhaps 10 to 1,000 primers that each yield a non-coding amplified sequence of 100 bases. If predicted coding sequences, such as those in SEQ ID NO:1, 3, 5, 7, 9, 11, 13, or 15, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining MEMX protein and/or nucleic acid expression as well as MEMX activity, in the context of a biological sample (e.g., blood, serum, cells, tissue) to thereby determine whether an individual is afflicted with a disease or disorder, or is at risk of developing a disorder, associated with aberrant MEMX expression or activity. The invention also provides for prognostic (or predictive) assays for determining whether an individual is at risk of developing a disorder associated with MEMX protein, nucleic acid expression or activity. For example, mutations in an MEMX gene can be assayed in a biological sample. Such assays can be used for prognostic or predictive purpose to thereby prophylactically treat an individual prior to the onset of a disorder characterized by or associated with MEMX protein, nucleic acid expression, or biological activity.

Another aspect of the invention provides methods for determining MEMX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of MEMX in clinical trials.

These and other agents are described in further detail in the following sections.

I. Diagnostic Assays

An exemplary method for detecting the presence or absence of MEMX in a biological sample involves obtaining a biological sample from a test subject and contacting the biological sample with a compound or an agent capable of detecting MEMX protein or nucleic acid (e.g., mRNA, genomic DNA) that encodes MEMX protein such that the presence of MEMX is detected in the biological sample. An agent for detecting MEMX mRNA or genomic DNA is a labeled nucleic acid probe capable of hybridizing to MEMX mRNA or genomic DNA. The nucleic acid probe can be, for example, a full-length MEMX nucleic acid, such as the nucleic acid of SEQ ID NO:1, 3, 5 7, 9, 11, 13, or 15, or a portion thereof, such as an oligonucleotide of at least 15, 30, 50, 100, 250 or 500 nucleotides in length and sufficient to specifically hybridize under stringent conditions to MEMX mRNA or genomic DNA. Other suitable probes for use in the diagnostic assays of the invention are described herein.

One agent for detecting MEMX protein is an antibody capable of binding to MEMX protein, preferably an antibody with a detectable label. Antibodies directed against a protein of the invention may be used in methods known within the art relating to the localization and/or quantitation of the protein (e.g., for use in measuring levels of the protein within appropriate physiological samples, for use in diagnostic methods, for use in imaging the protein, and the like). In a given embodiment, antibodies against the proteins, or derivatives, fragments, analogs or homologs thereof, that contain the antigen binding domain, are utilized as pharmacologically-active compounds.

An antibody specific for a protein of the invention can be used to isolate the protein by standard techniques, such as immunoaffinity chromatography or immunoprecipitation. Such an antibody can facilitate the purification of the natural protein antigen from cells and of recombinantly produced antigen expressed in host cells. Moreover, such an antibody can be used to detect the antigenic protein (e.g., in a cellular lysate or cell supernatant) in order to evaluate the abundance and pattern of expression of the antigenic protein. Antibodies directed against the protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect MEMX mRNA, protein, or genomic DNA in a biological sample in vitro as well as in vivo. For example, in vitro techniques for detection of MEMX mRNA include Northern hybridizations and in situ hybridizations. In vitro techniques for detection of MEMX protein include enzyme linked immunosorbent assays (ELISAs), Western blots, immunoprecipitations, and immunofluorescence. In vitro techniques for detection of MEMX genomic DNA include Southern hybridizations. Furthermore, in vivo techniques for detection of MEMX protein include introducing into a subject a labeled anti-MEMX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In one embodiment, the methods further involve obtaining a control biological sample from a control subject, contacting the control sample with a compound or agent capable of detecting MEMX protein, mRNA, or genomic DNA, such that the presence of MEMX protein, mRNA or genomic DNA is detected in the biological sample, and comparing the presence of MEMX protein, mRNA or genomic DNA in the control sample with the presence of MEMX protein, mRNA or genomic DNA in the test sample.

The invention also encompasses kits for detecting the presence of MEMX in a biological sample. For example, the kit can comprise: a labeled compound or agent capable of detecting MEMX protein or mRNA in a biological sample; means for determining the amount of MEMX in the sample; and means for comparing the amount of MEMX in the sample with a standard. The compound or agent can be packaged in a suitable container. The kit can further comprise instructions for using the kit to detect MEMX protein or nucleic acid.

II. Prognostic Assays

The diagnostic methods described herein can furthermore be utilized to identify subjects having or at risk of developing a disease or disorder associated with aberrant MEMX expression or activity. Such disorders for MEM1 include immunological conditions, viral infections, neurological disorders, Alzheimer's or Parkinson's Diseases, cancer (e.g., breast or neuroblastoma), nephrology, and female reproductive health. Such disorders for MEM4 include those involving the lung and/or brain (e.g., schizophrenia, or neuronal damage following head injury). Disorders for MEM5 include heart and other muscular disorders (e.g., arrhythmial), clotting deficiencies, and cobalamine deficiencies (e.g., pernicious anemia). Such disorders for MEM6 include those originating in dysregulation of glycogen metabolism (e.g., diabetes). Such disorders for MEM7 and MEM8 include vision-related disorders (e.g., keratomalacia), cancer, and other neoplastic pathologies.

For example, the assays described herein, such as the preceding diagnostic assays or the following assays, can be utilized to identify a subject having or at risk of developing a disorder associated with MEMX protein, nucleic acid expression or activity. Alternatively, the prognostic assays can be utilized to identify a subject having or at risk for developing a disease or disorder. Thus, the invention provides a method for identifying a disease or disorder associated with aberrant MEMX expression or activity in which a test sample is obtained from a subject and MEMX protein or nucleic acid (e.g., mRNA, genomic DNA) is detected, wherein the presence of MEMX protein or nucleic acid is diagnostic for a subject having or at risk of developing a disease or disorder associated with aberrant MEMX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant MEMX expression or activity. For example, such methods can be used to determine whether a subject can be effectively treated with an agent for a disorder. Thus, the invention provides methods for determining whether a subject can be effectively treated with an agent for a disorder associated with aberrant MEMX expression or activity in which a test sample is obtained and MEMX protein or nucleic acid is detected (e.g., wherein the presence of MEMX protein or nucleic acid is diagnostic for a subject that can be administered the agent to treat a disorder associated with aberrant MEMX expression or activity).

The methods of the invention can also be used to detect genetic lesions in an MEMX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an MEMX-protein, or the misexpression of the MEMX gene. For example, such genetic lesions can be detected by ascertaining the existence of at least one of: (i) a deletion of one or more nucleotides from an MEMX gene; (ii) an addition of one or more nucleotides to an MEMX gene; (iii) a substitution of one or more nucleotides of an MEMX gene, (iv) a chromosomal rearrangement of an MEMX gene; (v) an alteration in the level of a messenger RNA transcript of an MEMX gene, (vi) aberrant modification of an MEMX gene, such as of the methylation pattern of the genomic DNA, (vii) the presence of a non-wild-type splicing pattern of a messenger RNA transcript of an MEMX gene, (viii) a non-wild-type level of an MEMX protein, (ix) allelic loss of an MEMX gene, and (x) inappropriate post-translational modification of an MEMX protein. As described herein, there are a large number of assay techniques known in the art which can be used for detecting lesions in an MEMX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the MEMX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an MEMX gene under conditions such that hybridization and amplification of the MEMX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an MEMX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in MEMX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in MEMX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the MEMX gene and detect mutations by comparing the sequence of the sample MEMX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the MEMX gene include methods in which protection from cleavage agents is used to detect mismatched bases in RNA/RNA or RNA/DNA heteroduplexes. See, e.g., Myers, et al.; 1985. Science 230: 1242. In general, the art technique of “mismatch cleavage” starts by providing heteroduplexes of formed by hybridizing (labeled) RNA or DNA containing the wild-type MEMX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in MEMX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an MEMX sequence, e.g., a wild-type MEMX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in MEMX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control MEMX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an MEMX gene.

Furthermore, any cell type or tissue, preferably peripheral blood leukocytes, in which MEMX is expressed may be utilized in the prognostic assays described herein. However, any biological sample containing nucleated cells may be used, including, for example, buccal mucosal cells.

III. Pharmacogenomics

Agents, or modulators that have a stimulatory or inhibitory effect on MEMX activity (e.g., MEMX gene expression), as identified by a screening assay described herein can be administered to individuals to treat (prophylactically or therapeutically) disorders associated with aberrant MEMX activity. Such disorders for MEM1 include immunological conditions, viral infections, neurological disorders, Alzheimer's or Parkinson's Diseases, cancer (e.g., breast or neuroblastoma), nephrology, and female reproductive health. Such disorders for MEM4 include those involving the lung and/or brain (e.g., schizophrenia, or neuronal damage following head injury). Disorders for MEM5 include heart and other muscular disorders (e.g., arrhythmial), clotting deficiencies, and cobalamine deficiencies (e.g., pernicious anemia). Such disorders for MEM6 include those originating in dysregulation of glycogen metabolism (e.g., diabetes). Such disorders for MEM7 and MEM8 include vision-related disorders (e.g., keratomalacia), cancer, and other neoplastic pathologies.

In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of MEMX protein, expression of MEMX nucleic acid, or mutation content of MEMX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYMD6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of MEMX protein, expression of MEMX nucleic acid, or mutation content of MEMX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an MEMX modulator, such as a modulator identified by one of the exemplary screening assays described herein.

IV. Monitoring of Effects During Clinical Trials

Monitoring the influence of agents (e.g., drugs, compounds) on the expression or activity of MEMX (e.g., the ability to modulate aberrant cell proliferation and/or differentiation) can be applied not only in basic drug screening, but also in clinical trials. For example, the effectiveness of an agent determined by a screening assay as described herein to increase MEMX gene expression, protein levels, or upregulate MEMX activity, can be monitored in clinical trails of subjects exhibiting decreased MEMX gene expression, protein levels, or down-regulated MEMX activity. Alternatively, the effectiveness of an agent determined by a screening assay to decrease MEMX gene expression, protein levels, or down-regulate MEMX activity, can be monitored in clinical trails of subjects exhibiting increased MEMX gene expression, protein levels, or up-regulated MEMX activity. In such clinical trials, the expression or activity of MEMX and, preferably, other genes that have been implicated in, for example, a cellular proliferation or immune disorder can be used as a “read out” or markers of the immune responsiveness of a particular cell.

By way of example, and not of limitation, genes, including MEMX, that are modulated in cells by treatment with an agent (e.g., compound, drug or small molecule) that modulates MEMX activity (e.g., identified in a screening assay as described herein) can be identified. Thus, to study the effect of agents on cellular proliferation disorders, for example, in a clinical trial, cells can be isolated and RNA prepared and analyzed for the levels of expression of MEMX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of MEMX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an MEMX protein, mRNA, or genomic DNA in the pre-administration sample; (iii) obtaining one or more post-administration samples from the subject; (iv) detecting the level of expression or activity of the MEMX protein, mRNA, or genomic DNA in the post-administration samples; (v) comparing the level of expression or activity of the MEMX protein, mRNA, or genomic DNA in the pre-administration sample with the MEMX protein, mRNA, or genomic DNA in the post administration sample or samples; and (vi) altering the administration of the agent to the subject accordingly. For example, increased administration of the agent may be desirable to increase the expression or activity of MEMX to higher levels than detected, i.e., to increase the effectiveness of the agent. Alternatively, decreased administration of the agent may be desirable to decrease expression or activity of MEMX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant MEMX expression or activity. For example, disorders associated with aberrant MEM1 expression of activity include but are not limited to, viral infections, neurological disorders (e.g., Alzheimer's disease or Parkinson's disease), cancer (e.g., breast or neuroblastoma), and various renal disorders. Disorders associated with aberrant MEM2, MEM3, and MEM4 expression of activity include, but are not limited to, psychiatric diseases (e.g., schizophrenia) or reducing neuronal damage following head injury. Disorders associated with aberrant MEM5 expression include, but are not limited to, heart and other muscular disorders (e.g., arrhythmic disorders), clotting Factor XI in clotting deficiencies, and cobalamin-deficiencies (e.g., pernicious anemia). Disorders associated with aberrant MEM6 expression include, but are not limited to, glycogen-metabolism-related disorders (e.g., diabetes and related disorders). Disorders associated with aberrant MEM7 and MEM8 expression include, but are not limited to, vision-related disorders (e.g., keratomalacia) and cancer and/or similar neoplastic pathologies.

These methods of treatment will be discussed more fully, below.

I. Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

I. Prophylactic Methods

In one aspect, the invention provides a method for preventing, in a subject, a disease or condition associated with an aberrant MEMX expression or activity, by administering to the subject an agent that modulates MEMX expression or at least one MEMX activity. Subjects at risk for a disease that is caused or contributed to by aberrant MEMX expression or activity can be identified by, for example, any or a combination of diagnostic or prognostic assays as described herein. Administration of a prophylactic agent can occur prior to the manifestation of symptoms characteristic of the MEMX aberrancy, such that a disease or disorder is prevented or, alternatively, delayed in its progression. Depending upon the type of MEMX aberrancy, for example, an MEMX agonist or MEMX antagonist agent can be used for treating the subject. The appropriate agent can be determined based on screening assays described herein. The prophylactic methods of the invention are further discussed in the following subsections.

II. Therapeutic Methods

Another aspect of the invention pertains to methods of modulating MEMX expression or activity for therapeutic purposes. The modulatory method of the invention involves contacting a cell with an agent that modulates one or more of the activities of MEMX protein activity associated with the cell. An agent that modulates MEMX protein activity can be an agent as described herein, such as a nucleic acid or a protein, a naturally-occurring cognate ligand of a MEMX protein, a peptide, a MEMX peptidomimetic, or other small molecule. In one embodiment, the agent stimulates one or more MEMX protein activity. Examples of such stimulatory agents include active MEMX protein and a nucleic acid molecule encoding MEMX that has been introduced into the cell. In another embodiment, the agent inhibits one or more MEMX protein activity. Examples of such inhibitory agents include antisense MEMX nucleic acid molecules and anti-MEMX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a MEMX protein or nucleic acid molecule. In one embodiment, the method involves administering an agent (e.g., an agent identified by a screening assay described herein), or combination of agents that modulates (e.g., up-regulates or down-regulates) MEMX expression or activity. In another embodiment, the method involves administering a MEMX protein or nucleic acid molecule as therapy to compensate for reduced or aberrant MEMX expression or activity:

Stimulation of MEMX activity is desirable in situations in which MEMX is abnormally down-regulated and/or in which increased MEMX activity is likely to have a beneficial effect. One example of such a situation is where a subject has a disorder characterized by aberrant cell proliferation and/or differentiation (e.g., cancer or immune associated). Another example of such a situation is where the subject has an immunodeficiency disease (e.g., AIDS).

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of non-limiting example, from about 0.1 mg/kg body weight to about 50 mg/kg body weight. Common dosing frequencies may range, for example, from twice daily to once a week.

III. Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

### SPECIFIC EXAMPLES

### Example 1

### Real Time Quantitative (RTQ) PCR Evaluation of Expression of MEM5 in Various Cells and Tissues

The quantitative expression of MEM5 (Internal Identification 16418841) was assessed in normal and tumor samples by real time quantitative PCR (TAQMAN@) performed on a Perkin-Elmer Biosystems ABI PRISM® 7700 Sequence Detection System. In the Tables contained within this Example, the following abbreviations are used:

| | |
|---|---|
| ca. = carcinoma, | squam = squamous, |
| * = established from metastasis, | pl. eff = pl effusion = pleural effusion, |
| met = metastasis, | glio = glioma, |
| s cell var = small cell variant, | astro = astrocytoma, |
| non-s = non-sm = non-small, | neuro = neuroblastoma |

96 RNA samples were normalized to internal standards such as β-actin and GAPDH. RNA (˜50 ng total or ˜1 ng polyA+) was converted to cDNA using the TAQMAN® Reverse Transcription Reagents Kit (PE Biosystems; Foster City, Calif.; Catalog No. N808-0234) and random hexamers according to the manufacturer's protocol. Reactions were performed in 20 μl and incubated for 30 min. at 48° C. cDNA (5 μl) was then transferred to a separate plate for the TAQMAN® reaction using internal standards such as β-actin and GAPDH TAQMAN® Assay Reagents (PE Biosystems; Catalog Nos. 4310881E and 4310884E, respectively) and TAQMAN® Universal PCR Master Mix (PE Biosystems; Catalog No. 4304447) according to the manufacturer's protocol. Reactions were performed in 25 μl total reaction volume using the following parameters: 2 minutes at 50° C.; 10 minutes at 95° C.; 15 seconds at 95° C.; and 1 minute at 60° C. (40 cycles). Results were recorded as CT values (cycle at which a given sample crosses a threshold level of fluorescence) using a log scale, with the difference in RNA concentration between a given sample and the sample with the lowest CT value being represented as 2^{δCT}. The percent relative expression is then obtained by taking the reciprocal of this RNA difference and multiplying by 100. The average CT values obtained for B-actin and GAPDH were used to normalize RNA samples. The RNA sample generating the highest CT value required no further diluting, while all other samples were diluted relative to this sample according to their

β-actin/GAPDH average CT values.

Normalized RNA (5 μl) was converted to cDNA and analyzed via TAQMAN® using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (Version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58′-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′- and 3′-termini of the probe, respectively. Their final concentrations were: forward and reverse primers=900 nM each, and probe=200 nM.

The following PCR conditions were utilized. Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1×TaqMan™ PCR Master Mix for the PE Biosystems 7700, with 5 mM MgCl2, dNTPs (dA, dG, dC, dU at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 minutes; then 40 cycles of 95° C. for 15 seconds; 60° C. for 1 minute.

Two sample panels were employed in the present Example. Panel 1 is a 96 well plate (usually 2 control wells and 94 test samples) whose wells are contain RNA or cDNA isolated from various human cell lines that have been established from human malignant tissues (i.e., tumors). These cell lines have been extensively characterized by investigators in both academia and the commercial sector regarding their tumorgenicity, metastatic potential, drug resistance, invasive potential and other cancer-related properties. They serve as suitable tools for pre-clinical evaluation of anti-cancer agents and promising therapeutic strategies. RNA from these various human cancer cell lines was isolated by and procured from the Developmental Therapeutic Branch (DTB) of the National Cancer Institute (USA). Basic information regarding their biological behavior, gene expression, and resistance to various cytotoxic agents are provided by the DTB (http://dtp.nci.nih.gov/).

In addition, RNA or cDNA was obtained from various human tissues derived from human autopsies performed on deceased elderly people or sudden death victims (accidents, etc.). These tissues were ascertained to be free of disease and were purchased from various high quality commercial sources such as Clontech, Research Genetics, and Invitrogen.

RNA integrity from all samples was controlled for quality by visual assessment of agarose gel electrophoresis using 28S and 18S ribosomal RNA (rRNA) staining intensity ratio as a guide (2:1 to 2.5:1 28S:18S rRNA ratio) and the assuring the absence of low molecular weight RNAs indicative of degradation products.

Panel 2 is a 96 well plate (usually 2 control wells and 94 test samples) containing RNA or cDNA isolated from human tissue procured by surgeons working in close cooperation with the National Cancer Institute's Cooperative Human Tissue Network (CHTN) or the National Disease Research Initiative (NDRI). The tissues procured are derived from human malignancies and in cases where indicated many malignant tissues have “matched margins”. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e., immediately proximal) to the zone of surgery (designated “NAT”, for normal adjacent tissue). In addition, RNA or cDNA was obtained from various human tissues derived from human autopsies performed on deceased elderly people or sudden death victims (accidents, etc.). These tissue were ascertained to be free of disease and were purchased from various high quality commercial sources such as Clontech, Research Genetics, and Invitrogen.

Again, RNA integrity from all samples was controlled for quality by visual assessment of agarose gel electrophoresis using 28S and 18S rRNA staining intensity ratio as a guide (2:1 to 2.5:1 28S:18S ratio) and by assuring the absence of low molecular weight RNAs indicative of degradation products. Samples are quality controlled for genomic DNA contamination by reactions run in the absence of reverse transcriptase using probe and primer sets designed to amplify across the span of a single exon.

The following RTQ PCR of the MEM5 sequence (Internal Designation 16418841) utilzing Panel 1, is shown in Table 3, using the primer-probe set Ag765 designated in Table 2.

TABLE 2

| | | | Start | SEQ ID |
|---|---|---|---|---|
| Primers | Sequences | Length | Position | NO: |
| Forward | 5′-CCAACGTGAAGGGAGCTATAT-3′ | 21 | 923 | 17 |
| Probe | TET-5′-TGCTGACACCACTACACATGTCACAA-3′-TAMRA | 26 | 953 | 18 |
| Reverse | 5′-CCAGCCCCTAAAATTCTCATC-3′ | 21 | 986 | 19 |

TABLE 3

| | Rel. Expr., | Rel. Expr., | Rel. Expr., | | Rel. Expr., | Rel. Expr., | Rel. Expr., |
|---|---|---|---|---|---|---|---|
| | % | % | % | | % | % | % |
| Cell source | 1.2tm717t | 1.2tm917t | 1.2tm971t | Cell source | 1.2tm717t | 1.2tm917t | 1.2tm971t |
| Endothelial cells | 22.5 | 22.5 | 22.1 | Renal ca.786-0 | 17.6 | 17.6 | 5.3 |
| Endothelial cells | 2.0 | 2.0 | 1.3 | Renal Ca. A498 | 27.2 | 27.2 | 22.1 |
| (treated) | | | | | | | |
| Pancreas | 27.7 | 27.7 | 32.5 | Renal ca. RXF 393 | 2.8 | 2.8 | 2.5 |
| Pancreatic Ca. | 11.8 | 11.8 | 3.9 | Renal ca. ACHN | 8.7 | 8.7 | 6.9 |
| CAPAN2 | | | | | | | |
| Adrenal Gland (new | 12.5 | 12.5 | 14.9 | Renal ca. UO-31 | 16.6 | 16.6 | 4.5 |
| lot*) | | | | | | | |
| Thyroid | 24.8 | 24.8 | 18.7 | Renal ca. TK-10 | 20.3 | 20.3 | 9.1 |
| Salivary gland | 35.1 | 35.1 | 33.9 | Liver | 6.8 | 6.8 | 6.9 |
| Pituitary gland | 33.7 | 33.7 | 44.1 | Liver (fetal) | 12.9 | 12.9 | 12.8 |
| Brain (fetal) | 1.7 | 1.7 | 3.2 | Liver Ca. | 8.8 | 8.8 | 3.2 |
| | | | | (hepatoblast) HepG2 | | | |
| Brain (whole) | 11.9 | 11.9 | 13.0 | Lung | 11.8 | 11.8 | 10.7 |
| Brain (amygdala) | 2.9 | 2.9 | 4.4 | Lung (fetal) | 10.9 | 10.9 | 12.6 |
| Brain (cerebellum) | 6.6 | 6.6 | 10.0 | Lung ca. (small cell) | 26.2 | 26.2 | 16.5 |
| | | | | LX-1 | | | |
| Brain (hippocampus) | 6.7 | 6.7 | 8.9 | Lung ca. (small cell) | 28.7 | 28.7 | 9.2 |
| | | | | NCI-H69 | | | |
| Brain (thalamus) | 6.1 | 6.1 | 5.5 | Lung ca. (s.ceIl var.) | 12.5 | 12.5 | 7.7 |
| | | | | SHP-77 | | | |
| Cerebral Cortex | 7.6 | 7.6 | 0.0 | Lung ca. (large | 23.7 | 23.7 | 17.1 |
| | | | | cell)NCI-H460 | | | |
| Spinal cord | 7.1 | 7.1 | 10.7 | Lung ca. (non-sm. | 12.6 | 12.6 | 6.5 |
| | | | | cell) A549 | | | |
| CNS ca. (glio/astro) | 22.9 | 22.9 | 22.9 | Lung ca. (non-s.cell) | 7.3 | 7.3 | 6.0 |
| U87-MG | | | | NCI-H23 | | | |
| CNS ca. (glio/astro) | 33.5 | 33.5 | 25.9 | Lung ca (non-s.cell) | 11.5 | 11.5 | 0.0 |
| U-118-MG | | | | HOP-62 | | | |
| CNS ca. (astro) | 7.8 | 7.8 | 6.0 | Lung ca. (non-sl) | 44.1 | 44.1 | 24.7 |
| SW1783 | | | | NCI-H522 | | | |
| CNS ca.* (neuro; | 26.1 | 26.1 | 23.3 | Lung ca. (squam.) | 15.9 | 15.9 | 8.1 |
| met) SK-N-AS | | | | SW 900 | | | |
| CNS ca. (astro) SF- | 16.7 | 16.7 | 11.6 | Lung ca. (squam.) | 21.6 | 21.6 | 16.4 |
| 539 | | | | NCI-H596 | | | |
| CNS ca. (astro) | 19.6 | 19.6 | 12.5 | Mammary | 24.8 | 24.8 | 24.7 |
| SNB-75 | | | | gland | | | |
| CNS Ca. (glio) | 65.1 | 65.1 | 27.0 | Breast ca.* (pl. | 11.7 | 11.7 | 7.6 |
| SNB-19 | | | | effusion) MCF-7 | | | |
| CNS ca. (glio) U251 | 17.2 | 17.2 | 9.0 | Breast ca.* (pl.ef) | 14.3 | 14.3 | 13.4 |
| | | | | MDA-MB-231 | | | |
| CNS ca. (glio) SF- | 22.1 | 22.1 | 20.0 | Breast ca.* (p1. | 11.0 | 11.0 | 12.3 |
| 295 | | | | effusion) T47D | | | |
| Heart | 57.8 | 57.8 | 57.4 | Breast ca. BT-549 | 21.2 | 21.2 | 15.3 |
| Skeletal Muscle | 100.0 | 100.0 | 100.0 | Breast ca. MDA-N | 10.8 | 10.8 | 13.4 |
| (new lot*) | | | | | | | |
| Bone marrow | 7.2 | 7.2 | 7.2 | Ovary | 2.2 | 2.2 | 0.3 |
| Thymus | 5.6 | 5.6 | 4.2 | Ovarian ca. | 15.5 | 15.5 | 12.5 |
| | | | | OVCAR-3 | | | |
| Spleen | 6.0 | 6.0 | 7.4 | Ovarian ca. | 5.0 | 5.0 | 5.0 |
| | | | | OVCAR-4 | | | |
| Lymph node | 7.0 | 7.0 | 7.7 | Ovarian ca. | 12.2 | 12.2 | 6.4 |
| | | | | OVCAR-5 | | | |
| Colorectal | 2.2 | 2.2 | 1.3 | Ovarian ca. | 15.3 | 15.3 | 5.6 |
| | | | | OVCAR-8 | | | |
| Stomach | 12.4 | 12.4 | 15.8 | Ovarian ca. IGROV-1 | 20.5 | 20.5 | 12.9 |
| Small intestine | 23.5 | 23.5 | 17.8 | Ovarian ca.* | 30.6 | 30.6 | 26.6 |
| | | | | (ascites) SK-OV-3 | | | |
| Colon ca. SW480 | 15.6 | 15.6 | 6.5 | Uterus | 9.2 | 9.2 | 6.3 |
| Colon ca.* (SW480 | 38.4 | 38.4 | 15.9 | Placenta | 13.7 | 13.7 | 15.2 |
| met)SW620 | | | | | | | |
| Colon ca. HT29 | 8.5 | 8.5 | 3.1 | Prostate | 20.6 | 20.6 | 16.6 |
| Colon ca. HCT-116 | 20.9 | 20.9 | 10.5 | Prostate ca.* (bone | 23.7 | 23.7 | 15.5 |
| | | | | met)PC-3 | | | |
| Colon ca. CaCo-2 | 11.5 | 11.5 | 4.5 | Testis | 29.5 | 29.5 | 23.8 |
| 83219 CC Well to | 3.9 | 3.9 | 0.4 | Melanoma | 12.0 | 12.0 | 8.4 |
| Mod Diff | | | | Hs688(A).T | | | |
| (ODO3866) | | | | | | | |
| Colon ca. HCC-2998 | 35.4 | 35.4 | 21.6 | Melanoma* (met) | 15.9 | 15.9 | 11.0 |
| | | | | Hs688(B).T | | | |
| Gastric ca.* (liver | 25.5 | 25.5 | 21.0 | Melanoma UACC- | 1.9 | 1.9 | 1.0 |
| met) NCI-N87 | | | | 62 | | | |
| Bladder | 23.0 | 23.0 | 22.5 | Melanoma M14 | 14.4 | 14.4 | 1.7 |
| Trachea | 9.4 | 9.4 | 10.7 | Melanoma LOX | 8.3 | 8.3 | 2.1 |
| | | | | IMVI | | | |
| Kidney | 12.9 | 12.9 | 13.1 | Melanoma* (met) | 14.1 | 14.1 | 11.2 |
| | | | | SK-MEL-5 | 2.1 | 2.1 | 1.0 |
| Kidney (fetal) | 19.0 | 19.0 | 8.3 | Adipose | | | |

The results in Table 3 show that the sequence of MEM5 is expressed in a wide variety of normal and cancer cell lines. With relation to normal tissues, it is more highly expressed in certain brain tumors such as CNS ca. (glio) SNB-19, colon cancer such as Colon ca.* (SW480 met)SW620, and lung cancer such as Lung ca. (non-s.cl) NCI-H522.

Additional results for MEM5 which were obtained using Panel 2 are shown in Table 4.

TABLE 4

| | Rel. | | Rel. |
|---|---|---|---|
| | Expr., % | | Expr., % |
| Tissue source | 2tm972t | Tissue source | 2tm972t |
| 83786 Kidney Ca, Nuclear grade 2 (OD04338) | 15.4 | 87492 Ovary Cancer (OD04768-07) | 78.5 |
| 83219 CC Well to Mod Diff (ODO3866) | 2.1 | 87493 Ovary NAT (OD04768-08) | 11.7 |
| 83220 CC NAT (ODO3866) | 11.7 | Bladder Cancer INVITROGEN A302173 | 7.7 |
| 83221 CC Gr. 2 rectosigmoid (ODO3868) | 11.7 | Bladder Cancer Research Genetics RNA 1023 | 0.0 |
| 83222 CC NAT (ODO3868) | 3.4 | Breast Cancer Clontech 9100266 | 2.8 |
| 83235 CC Mod Diff (ODO3920) | 28.9 | Breast Cancer INVITROGEN A209073 | 1.9 |
| 83236 CC NAT (ODO3920) | 30.8 | Breast Cancer Res. Gen. 1024 | 21.8 |
| 83237 CC Gr. 2 ascend colon (ODO3921) | 10.4 | Breast NAT Clontech 9100265 | 0.1 |
| 83238 CC NAT (ODO3921) | 1.6 | Breast NAT INVITROGEN A2090734 | 5.1 |
| 83239 Lung Met to Muscle (ODO4286) | 1.5 | GENPAK Breast Cancer 064006 | 17.3 |
| 83240 Muscle NAT (ODO4286) | 18.4 | Gastric Cancer Clontech 9060395 | 14.8 |
| 83241 CC from Partial Hepatectomy (ODO4309) | 9.6 | Gastric Cancer Clontech 9060397 | 3.0 |
| 83242 Liver NAT (ODO4309) | 22.1 | Gastric Cancer GENPAK 064005 | 48.6 |
| 83255 Ocular Mel Met to Liver (ODO4310) | 54.7 | Kidney Cancer Clontech 8120607 | 0.0 |
| 83256 Liver NAT (ODO4310) | 25.2 | Kidney Cancer Clontech 8120613 | 0.3 |
| 83787 Kidney NAT (OD04338) | 32.8 | Kidney Cancer Clontech 9010320 | 0.4 |
| 83788 Kidney Ca Nuclear grade 1/2 (OD04339) | 59.1 | Kidney NAT Clontech 8120608 | 0.1 |
| 83789 Kidney NAT (OD04339) | 50.4 | Kidney NAT Clontech 8120614 | 0.0 |
| 83790 Kidney Ca, Clear cell type (OD04340) | 100.0 | Kidney NAT Clontech 9010321 | 0.1 |
| 83791 Kidney NAT (OD04340) | 39.2 | Liver Cancer GENPAK 064003 | 23.8 |
| 83792 Kidney Ca, Nuclear grade 3 (OD04348) | 24.2 | Liver Cancer Research Genetics RNA 1025 | 0.8 |
| 83793 Kidney NAT (OD04348) | 25.4 | Liver Cancer Research Genetics RNA 1026 | 0.1 |
| 84136 Lung Malignant Cancer (OD03126) | 5.5 | NAT Stomach Clontech 9060359 | 14.3 |
| 84137 Lung NAT (OD03126) | 9.7 | NAT Stomach Clontech 9060394 | 11.4 |
| 84138 Lung NAT (OD04321) | 4.8 | NAT Stomach Clontech 9060396 | 5.4 |
| 84139 Melanoma Mets to Lung (OD04321) | 26.2 | Normal Bladder GENPAK 061001 | 18.8 |
| 84140 Prostate Cancer (OD04410) | 26.8 | Normal Breast GENPAK 061019 | 5.4 |
| 84141 Prostate NAT (OD04410) | 41.8 | Normal Colon GENPAK 061003 | 17.2 |
| 84871 Lung Cancer (OD04404) | 2.0 | Normal Kidney GENPAK 061008 | 11.8 |
| 84872 Lung NAT (OD04404) | 0.0 | Normal Liver GENPAK 061009 | 26.4 |
| 84875 Lung Cancer (OD04565) | 55.9 | Normal Lung GENPAK 061010 | 10.9 |
| 84877 Breast Cancer (OD04566) | 7.2 | Normal Ovary Res. Gen. | 0.6 |
| 85950 Lung Cancer (OD04237-01) | 7.0 | Normal Prostate Clontech A+ 6546-1 | 3.9 |
| 85970 Lung NAT (OD04237-02) | 12.2 | Normal Stomach GENPAK 061017 | 12.9 |
| 85973 Kidney Cancer (OD04450-01) | 20.6 | Normal Thyroid Clontech A+ 6570-1** | 10.3 |
| 85974 Kidney NAT (OD04450-03) | 40.6 | Normal Uterus GENPAK 061018 | 12.6 |
| 85975 Breast Cancer (OD04590-01) | 31.6 | Ovarian Cancer GENPAK 064008 | 9.3 |
| 85976 Breast Cancer Mets (OD04590-03) | 39.5 | Paired Liver Cancer Tissue RNA 6004-T | 0.4 |
| 87070 Breast Cancer Metastasis (OD04655-05) | 26.6 | Paired Liver Cancer Tissue RNA 6005-T | 1.3 |
| 87071 Bladder Cancer (OD04718-01) | 28.3 | Paired Liver lissue RNA 6004-N | 9.0 |
| 87072 Bladder Normal Adjacent (OD04718-03) | 1.8 | Paired Liver Tissue Research Genetics RNA | 0.8 |
| | | 6005-N | |
| 87073 Prostate Cancer (OD04720-01) | 52.5 | Thyroid Cancer GENPAK 064010 | 17.8 |
| 87074 Prostate NAT (OD04720-02) | 6.2 | Thyroid Cancer INVITROGEN A302152 | 27.7 |
| 87472 Colon mets to lung (OD04451-01) | 6.7 | Thyroid NAT INVITROGEN A302153 | 23.2 |
| 87473 Lung NAT (OD04451-02) | 4.3 | Uterus Cancer GENPAK 064011 | 29.1 |
| 87474 Kidney Cancer (OD04622-01) | 7.5 | genomic DNA control | 0.3 |
| 87475 Kidney NAT (OD04622-03) | 1.7 | 87492 Ovary Cancer (0004768-07) | 78.5 |

The results shown in Table 4 indicate that MEM5 is expressed preferentially in certain tumor samples compared to the adjacent noncancerous tissue. These tumors include a liver metastasis, a kidney tumor, a prostate cancer, and an ovarian cancer. In addition there is high expression in additional tumor tissues that have no matching normal tissue in the panel.

Accordingly, the results in Tables 3 and 4 suggests that MEM5 may serve as a diagnostic probe for certain specific cancer types.

### Example 2

### Real Time Quantitative (RTQ) PCR Evaluation of Expression of MEM7 in Various Cells and Tissues

The quantitative expression of MEM7 (Internal Identification AC018653_A) was assessed in normal and tumor samples by real time quantitative PCR (TAQMAN®) performed on a Perkin-Elmer Biosystems ABI PRISM® 7700 Sequence Detection System. In the Tables contained within this Example, the following abbreviations are used:

| | |
|---|---|
| ca. = carcinoma, | squam = squamous, |
| * = established from metastasis, | pl. eff = pl effusion = pleural effusion, |
| met = metastasis, | glio = glioma, |
| s cell var = small cell variant, | astro = astrocytoma, |
| non-s = non-sm = non-small, | neuro = neuroblastoma |

96 RNA samples were normalized to internal standards such as β-actin and GAPDH. RNA (˜50 ng total or ˜1 ng polyA+) was converted to cDNA using the TAQMAN® Reverse Transcription Reagents Kit (PE Biosystems; Foster City, Calif.; Catalog No. N808-0234) and random hexamers according to the manufacturer's protocol. Reactions were performed in 20 μl and incubated for 30 min. at 48° C. cDNA (5 μl) was then transferred to a separate plate for the TAQMAN® reaction using internal standards such as β-actin and GAPDH TAQMAN® Assay Reagents (PE Biosystems; Catalog Nos. 4310881E and 4310884E, respectively) and TAQMAN® Universal PCR Master Mix (PE Biosystems; Catalog No. 4304447) according to the manufacturer's protocol. Reactions were performed in 25 μl total reaction volume using the following parameters: 2 minutes at 50° C.; 10 minutes at 95° C.; 15 seconds at 95° C.; and 1 minute at 60° C. (40 cycles). Results were recorded as CT values (cycle at which a given sample crosses a threshold level of fluorescence) using a log scale, with the difference in RNA concentration between a given sample and the sample with the lowest CT value being represented as 2^{δCT}. The percent relative expression is then obtained by taking the reciprocal of this RNA difference and multiplying by 100. The average CT values obtained for B-actin and GAPDH were used to normalize RNA samples. The RNA sample generating the highest CT value required no further diluting, while all other samples were diluted relative to this sample according to their

β-actin/GAPDH average CT values.

Normalized RNA (5 μl) was converted to cDNA and analyzed via TAQMAN® using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (Version I for Apple Computer's Macintosh Power PC) or a similar algorithm using the target sequence as input. Default settings were used for reaction conditions and the following parameters were set before selecting primers: primer concentration=250 nM, primer melting temperature (Tₘ)

range=58′-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′- and 3′-termini of the probe, respectively. Their final concentrations were: forward and reverse primers=900 nM each, and probe=200 nM.

The following PCR conditions were utilized. Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1×TaqMan™ PCR Master Mix for the PE Biosystems 7700, with 5 mM MgCl2, dNTPs (dA, dG, dC, dU at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 minutes; then 40 cycles of 95° C. for 15 seconds; 60° C. for 1 minute.

Two sample panels were employed in the present Example. Panel 1 is a 96 well plate (usually 2 control wells and 94 test samples) whose wells are contain RNA or cDNA isolated from various human cell lines that have been established from human malignant tissues (i.e., tumors). These cell lines have been extensively characterized by investigators in both academia and the commercial sector regarding their tumorgenicity, metastatic potential, drug resistance, invasive potential and other cancer-related properties. They serve as suitable tools for pre-clinical evaluation of anti-cancer agents and promising therapeutic strategies. RNA from these various human cancer cell lines was isolated by and procured from the Developmental Therapeutic Branch (DTB) of the National Cancer Institute (USA). Basic information regarding their biological behavior, gene expression, and resistance to various cytotoxic agents are provided by the DTB (http://dtp.nci.nih.gov/).

In addition, RNA or cDNA was obtained from various human tissues derived from human autopsies performed on deceased elderly people or sudden death victims (accidents, etc.). These tissues were ascertained to be free of disease and were purchased from various high quality commercial sources such as Clontech, Research Genetics, and Invitrogen.

RNA integrity from all samples was controlled for quality by visual assessment of agarose gel electrophoresis using 28S and 18S ribosomal RNA (rRNA) staining intensity ratio as a guide (2:1 to 2.5:1 28S:18S rRNA ratio) and the assuring the absence of low molecular weight RNAs indicative of degradation products.

Panel 2 is a 96 well plate (usually 2 control wells and 94 test samples) containing RNA or cDNA isolated from human tissue procured by surgeons working in close cooperation with the National Cancer Institute's Cooperative Human Tissue Network (CHTN) or the National Disease Research Initiative (NDRI). The tissues procured are derived from human malignancies and in cases where indicated many malignant tissues have “matched margins”. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e., immediately proximal) to the zone of surgery (designated “NAT”, for normal adjacent tissue). In addition, RNA or cDNA was obtained from various human tissues derived from human autopsies performed on deceased elderly people or sudden death victims (accidents, etc.). These tissue were ascertained to be free of disease and were purchased from various high quality commercial sources such as Clontech, Research Genetics, and Invitrogen.

Again, RNA integrity from all samples was controlled for quality by visual assessment of agarose gel electrophoresis using 28S and 18S rRNA staining intensity ratio as a guide (2:1 to 2.5:1 28S:18S ratio) and by assuring the absence of low molecular weight RNAs indicative of degradation products. Samples are quality controlled for genomic DNA contamination by reactions run in the absence of reverse transcriptase using probe and primer sets designed to amplify across the span of a single exon.

The following RTQ PCR of the MEM7 sequence (Internal Identification AC018653_A) utilzing Panel 1, is shown in Table 6, using the primer-probe set Ag 1387 designated in Table 5.

TABLE 5

| | | | Start | SEQ ID |
|---|---|---|---|---|
| Primers | Sequences | Length | Position | NO: |
| Forward | 5′-CTGAAACCTTCATCCACACAAT-3′ | 22 | 18 | 20 |
| Probe | TET-5′-TCACTGGCTACTACCGCTTTGTCTCG-3′-TAMRA | 26 | 51 | 21 |
| Reverse | 5′-GCAGGTAGTCCTCCATGTTCTT-3′ | 22 | 80 | 22 |

TABLE 6

| | Rel. Expr., | | Rel. Expr., |
|---|---|---|---|
| | %, | | %, |
| Cell source | 1.2tm1615t | Cell source | 1.2tm1615t |
| Endothelial cells | 0.1 | Renal ca. 786-0 | 0.2 |
| Endothelial cells (treated) | 0.6 | Renal ca. A498 | 0.7 |
| Pancreas | 0.0 | Renal ca. RXF 393 | 0.3 |
| Pancreatic ca. CAPAN 2 | 0.1 | Renal ca. ACHN | 0.7 |
| Adrenal Gland (new lot*) | 0.4 | Renal ca. UO-31 | 0.5 |
| Thyroid | 0.0 | Renal ca. TK-10 | 0.3 |
| Salavary gland | 0.7 | Liver | 12.7 |
| Pituitary gland | 0.0 | Liver (fetal) | 1.4 |
| Brain (fetal) | 0.0 | Liver ca. (hepatoblast) HepG2 | 1.7 |
| Brain (whole) | 0.0 | Lung | 0.1 |
| Brain (amygdala) | 0.0 | Lung (fetal) | 0.3 |
| Brain (cerebellum) | 0.0 | Lung ca. (small cell) LX-1 | 1.1 |
| Brain (hippocampus) | 0.1 | Lung ca. (small cell) NCI-H69 | 0.7 |
| Brain (thalamus) | 0.1 | Lung ca. (s. cell var.) SHP-77 | 0.0 |
| Cerebral Cortex | 0.2 | Lung ca. (large cell)NCI-H460 | 1.7 |
| Spinal cord | 0.0 | Lung ca. (non-sm. cell) A549 | 0.6 |
| CNS ca. (glio/astro) U87-MG | 0.1 | Lung ca. (non-s. cell) NCI-H23 | 1.3 |
| CNS ca. (glio/astro) U-118-MG | 0.1 | Lung ca (non-s. cell) HOP-62 | 0.6 |
| CNS ca. (astro) SW1783 | 0.1 | Lung Ca. (non-s. cl) NCI-H522 | 0.9 |
| CNS ca.* (neuro; met) SK-N-AS | 0.1 | Lung ca. (squam.) SW 900 | 0.5 |
| CNS ca. (astro) SF-539 | 0.3 | Lung ca. (squam.) NCI-H596 | 0.3 |
| CNS ca. (astro) SNB-75 | 0.0 | Mammary gland | 0.3 |
| CNS ca. (glio) SNB-19 | 0.1 | Breast ca.* (pl. effusion) MCF-7 | 0.0 |
| CNS ca. (glio) U251 | 0.0 | Breast ca.* (pl. ef) MDA-MB-231 | 0.0 |
| CNS ca. (glio) SF-295 | 0.3 | Breast ca.* (pl. effusion) T47D | 0.1 |
| Heart | 0.3 | Breast ca. BT-549 | 0.0 |
| Skeletal Muscle (new lot*) | 0.0 | Breast ca. MDA-N | 0.1 |
| Bone marrow | 0.2 | Ovary | 0.7 |
| Thymus | 0.4 | Ovarian ca. OVCAR-3 | 0.2 |
| Spleen | 1.5 | Ovarian ca. OVCAR-4 | 0.2 |
| Lymph node | 1.3 | Ovarian ca. OVCAR-5 | 1.3 |
| Colorectal | 0.0 | Ovarian ca. OVCAR-8 | 0.3 |
| Stomach | 0.9 | Ovarian ca. IGROV-1 | 0.3 |
| Small intestine | 1.8 | Ovarian ca.* (ascites) SK-OV-3 | 0.4 |
| Colon ca. SW480 | 0.1 | Uterus | 1.0 |
| Colon ca.* (SW480 met)SW620 | 0.3 | Plancenta | 0.0 |
| Colon ca. HT29 | 0.3 | Prostate | 0.9 |
| Colon ca. HCT-116 | 0.3 | Prostate ca.* (bone met)PC-3 | 0.4 |
| Colon ca. CaCo-2 | 0.5 | Testis | 0.0 |
| 83219 CC Well to Mod Diff (ODO3866) | 0.2 | Melanoma Hs688(A).T | 0.1 |
| Colon ca. HCC-2998 | 1.5 | Melanoma* (met) Hs688(B).T | 0.1 |
| Gastric ca.* (liver met) NCI-N87 | 0.7 | Melanoma UACC-62 | 0.1 |
| Bladder | 2.5 | Melanoma M14 | 0.0 |
| Trachea | 0.1 | Melanoma LOXIMVI | 0.0 |
| Kidney | 100.0 | Melanoma* (met) SK-MEL-5 | 0.0 |
| Kidney (fetal) | 0.3 | Adipose | 1.7 |

Additionally, the expression of sequence MEM7 was also evaluated using the same primer-probe set, Ag1387, on Panel 2. The results are shown in Table 7.

TABLE 7

| | Rel. | | Rel. |
|---|---|---|---|
| | Expr., %, | | Expr., %, |
| Tissue Source | 2tm515f | Tissue Source | 2tm515f |
| 83786 Kidney Ca, Nuclear grade 2 (OD04338) | 9.9 | 87492 Ovary Cancer (OD04768-07) | 17.7 |
| 83219 CC Well to Mod Diff (ODO3866) | 3.7 | 87493 Ovary NAT (OD04768-08) | 9.0 |
| 83220 CC NAT (ODO3866) | 3.7 | Bladder Cancer INVITROGEN A302173 | 7.9 |
| 83221 CC Gr. 2 rectosigmoid (ODO3868) | 3.4 | Bladder Cancer Research Genetics RNA 1023 | 4.0 |
| 83222 CC NAT (ODO3868) | 1.3 | Breast Cancer Clontech 9100266 | 6.1 |
| 83235 CC Mod Diff (ODO3920) | 10.6 | Breast Cancer INVITROGEN A209073 | 6.6 |
| 83236 CC NAT (ODO3920) | 5.0 | Breast Cancer Res. Gen. 1024 | 9.9 |
| 83237 CC Gr. 2 ascend colon (ODO3921) | 4.5 | Breast NAT Clontech 9100265 | 3.8 |
| 83238 CC NAT (ODO3921) | 2.3 | Breast NAT INVITROGEN A2090734 | 8.0 |
| 83239 Lung Met to Muscle (ODO4286) | 2.6 | GENPAK Breast Cancer 064006 | 12.8 |
| 83240 Muscle NAT (ODO4286) | 6.4 | Gastric Cancer Clontech 9060395 | 5.8 |
| 83241 CC from Partial Hepatectomy (ODO4309) | 7.1 | Gastric Cancer Clontech 9060397 | 6.3 |
| 83242 Liver NAT (ODO4309) | 80.1 | Gastric Cancer GENPAK 064005 | 12.5 |
| 83255 Ocular Mel Met to Liver (ODO4310) | 1.7 | Kidney Cancer Clontech 8120607 | 8.3 |
| 83256 Liver NAT (ODO4310) | 51.4 | Kidney Cancer Clontech 8120613 | 1.0 |
| 83787 Kidney NAT (OD04338) | 11.5 | Kidney Cancer Clontech 9010320 | 3.7 |
| 83788 Kidney Ca Nuclear grade 1/2 (OD04339) | 26.4 | Kidney NAT Clontech 8120608 | 4.4 |
| 83789 Kidney NAT (OD04339) | 10.7 | Kidney NAT Clontech 8120614 | 2.9 |
| 83790 Kidney Ca, Clear cell type (OD04340) | 12.0 | Kidney NAT Clontech 9010321 | 3.1 |
| 83791 Kidney NAT (OD04340) | 9.2 | Liver Cancer GENPAK 064003 | 23.5 |
| 83792 Kidney Ca, Nuclear grade 3 (OD04348) | 6.7 | Liver Cancer Research Genetics RNA 1025 | 92.7 |
| 83793 Kidney NAT (OD04348) | 12.9 | Liver Cancer Research Genetics RNA 1026 | 16.6 |
| 84136 Lung Malignant Cancer (OD03126) | 4.7 | NAT Stomach Clontech 9060359 | 7.1 |
| 84137 Lung NAT (OD03126) | 9.7 | NAT Stomach Clontech 9060394 | 7.2 |
| 84138 Lung NAT (OD04321) | 4.7 | NAT Stomach Clontech 9060396 | 3.4 |
| 84139 Melanoma Mets to Lung (OD04321) | 5.1 | Normal Bladder GENPAK 061001 | 13.0 |
| 84140 Prostate Cancer (OD04410) | 7.0 | Normal Breast GENPAK 061019 | 8.1 |
| 84141 Prostate NAT (OD04410) | 11.0 | Normal Colon GENPAK 061003 | 5.2 |
| 84871 Lung Cancer (OD04404) | 4.4 | Normal Kidney GENPAK 061008 | 4.8 |
| 84872 Lung NAT (OD04404) | 4.3 | Normal Liver GENPAK 061009 | 100.0 |
| 84875 Lung Cancer (OD04565) | 9.3 | Normal Lung GENPAK 061010 | 5.7 |
| 84877 Breast Cancer (OD04566) | 7.8 | Normal Ovary Res. Gen. | 7.0 |
| 85950 Lung Cancer (OD04237-01) | 6.2 | Normal Prostate Clontech A+ 6546-1 | 4.8 |
| 85970 Lung NAT (OD04237-02) | 5.1 | Normal Stomach GENPAK 061017 | 6.4 |
| 85973 Kidney Cancer (OD04450-01) | 13.4 | Normal Thyroid Clontech A+ 6570-1** | 6.1 |
| 85974 Kidney NAT (OD04450-03) | 6.3 | Normal Uterus GENPAK 061018 | 3.0 |
| 85975 Breast Cancer (OD04590-01) | 6.3 | Ovarian Cancer GENPAK 064008 | 11.7 |
| 85976 Breast Cancer Mets (OD04590-03) | 7.1 | Paired Liver Cancer Tissue RNA 6004-T | 54.0 |
| 87070 Breast Cancer Metastasis (OD04655-05) | 9.4 | Paired Liver Cancer Tissue RNA 6005-T | 18.4 |
| 87071 Bladder Cancer (OD04718-01) | 5.3 | Paired Liver Tissue RNA 6004-N | 26.1 |
| 87072 Bladder Normal Adjacent (OD04718-03) | 4.8 | Paired Liver Tissue Genetics RNA 6005-N | 55.1 |
| 87073 Prostate Cancer (OD04720-01) | 13.1 | Thyroid Cancer GENPAK 064010 | 3.4 |
| 87074 Prostate NAT (OD04720-02) | 10.4 | Thyroid Cancer INVITROGEN A302152 | 10.7 |
| 87472 Colon mets to lung (OD04451-01) | 9.5 | Thyroid NAT INVITROGEN A302153 | 7.7 |
| 87473 Lung NAT (OD04451-02) | 5.6 | Uterus Cancer GENPAK 064011 | 10.6 |
| 87474 Kidney Cancer (OD04622-01) | 27.4 | Genomic DNA control | 0.6 |
| 87475 Kidney NAT (OD04622-03) | 7.1 | | |

The results for MEM7 indicate expression primarily in normal kidney and lung tissue, and, for certain tumors but not all, in normal tissue adjacent to certain tumors in these organs. These results suggest that MEM7 may be used to distinguish normal from cancerous tissue.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated polynucleotide comprising a nucleic acid sequence encoding a polypeptide of SEQ ID NO:2.

2. An isolated polynucleotide comprising a nucleic acid sequence encoding the mature form of the polypeptide of SEQ ID NO:2.

3. An isolated polynucleotide comprising a nucleic acid sequence of SEQ ID NO:1.

4. An isolated polynucleotide consisting of a nucleic acid sequence of SEQ ID NO:1.

5. An isolated polynucleotide comprising a nucleic acid sequence encoding the complement of a polynucleotide of SEQ ID NO:1.

6. A vector comprising the nucleic acid sequence of claim 1.

7. The vector of claim 6, further comprising a promoter operably-linked to said nucleic acid molecule.

8. A cell comprising the vector of claim 6.

9. A pharmaceutical composition comprising the polynucleotide of claim 1 and a pharmaceutically-acceptable carrier.

10. An isolated polynucleotide consisting of a nucleic acid sequence encoding a polypeptide of SEQ ID NO:2.

11. An isolated polynucleotide consisting of a nucleic acid sequence encoding a mature form of the polypeptide of SEQ ID NO:2.

12. A kit comprising on one or more containers the pharmaceutical composition of claim 9.
